(19) United States
(12) Patent Application Publication
(10) Pub. No.: US 2013/0309664 A1
(43) Pub. Date: Nov. 21, 2013

(54) HEAT SHOCK PROTEINS AS AUTOANTIGENS

(71) Applicant: University of Pittsburgh - of the Commonwealth Sy, (US)

(72) Inventor: Steven R. Duncan, Pittsburgh, PA (US)

(73) Assignee: University of Pittsburgh- of the Commonwealth System of Higher Education, Pittsburgh, PA (US)

(21) Appl. No.: 13/673,022

(22) Filed: Nov. 9, 2012

Related U.S. Application Data
(63) Continuation of application No. PCT/US2011/036302, filed on May 12, 2011.

(51) Int. Cl. G01N 33/53

(52) U.S. Cl. CPC G01N 33/53
USPC 435/6.11; 435/7.92; 435/6.12

(57) ABSTRACT

The present invention relates to methods of diagnosing and/or determining a prognosis for various diseases, including lung diseases such as idiopathic pulmonary fibrosis, chronic obstructive lung disease, and emphysema as well as osteoporosis, comprising detecting the presence of autoantibodies specific for heat shock proteins in a subject.

### PRIORITY CLAIM

This application is a continuation of International Application Serial No. PCT/US2011/036302 filed May 12, 2011, and claims priority to U.S. Provisional Application Ser. No. 61/334,049 filed May 12, 2010 and U.S. Provisional Application Ser. No. 61/334,992 filed May 14, 2010, the contents of each of which are incorporated by reference in their entireties herein.

### GRANT INFORMATION

This invention was made with government support under grant numbers HL073241 and HL084948 awarded by the National Institutes of Health. The government has certain rights in the invention.

### 1. INTRODUCTION

The present invention relates to methods of diagnosing and/or determining a prognosis for various diseases, including lung diseases such as idiopathic pulmonary fibrosis, chronic obstructive lung disease, and emphysema as well as osteoporosis, comprising detecting the presence of autoantibodies specific for heat shock proteins in a subject.

### 2. BACKGROUND OF THE INVENTION

### 2.1 Autoimmunity

Autoimmunity is an adaptive immune response that is defined by demonstrable B-cell and/or T-cell reactivity against one or more self-epitopes (Duncan, 2010, Am T Respir Crit Care Med. 181(1)). B-cell autoimmunity is typified by production of self-avid immunoglobulin (Ig). T-cell autoimmunity is evidenced by proliferation induced by self-antigens. Low level autoimmunity is ubiquitous and probably important for immune system development and homeostasis (Elkon, et al., 2008, Nat Clin Pract Rheumatol 4:491-498). Under certain conditions, however, autoimmune responses become dysregulated and/or exaggerated and cause disease (Ermann, et al., 2001, Nat Immunol; 2:759-761) (Marrack, et al., 2001, Nat Immunol 7:899-905) (Lipsky, 2001, Nat Immunol 2:764-766).

Clinical features of autoimmune syndromes share similarities with those of smoking-related lung diseases (Duncan, 2010, Am J Respir Crit Care Med 181(1):4-5) (Agusti, et al., 2003, Thorax 58:832-4). Both have variable susceptibilities (e.g., lung disease severities differ between individuals with identical smoking histories, Mannino, 2002, Chest 121:121 S-6S; Burst et al., 2007 Lancet 370:741-750). Familial predilections are evident in both entities, (Burrows, et al., 1965, Am Rev Respir Dis, 91:665-678; Kurzius-Spencer, et al., 2001, Am J Respir Crit Care Med 164:1261-1265) and both are also frequently associated with systemic abnormalities (Chatila et al., 2008, Proceedings of the American Thoracic Society 5:549-555; Agusti, 2005, Proceedings of the American Thoracic Society 2:367-370; Decramer, et al., 2008, COPD 5:235-256; McAllister, et al., 2007, Am J Resp Crit Care Med 176:1208-1214; Sabit, et al., 2007, Am J Respir Crit Care Med 175:1259-1265; de Vries, et al., 2005, Eur Respir J 25:879-884; Bolton, et al., 2004, Am J Resp Crit Care Med 170:1286-1293; Bon, et al., 2011 Am J Respir Crit Care Med 183:885-90; Wilson, et al., 2008, Am J Resp Crit Care Med 178:738-744). Furthermore, autoimmune diseases and emphysema/COPD show persistent inflammation (and clinical progression) despite removal of the inciting agent (e.g., smoking cessation; Retamales, et al., 2001, Am J Respir Critical Care Med 164:469-73). Finally, both emphysema/COPD and autoimmune diseases may only be partially, if at all, responsive to nonspecific treatments (e.g., glucocorticoids), and relapses are common (Perosa, et al., 2010, J Intern Med 267:260-77; Erickson, et al., 1979, Mayo Clin Proc 54:714-720; Khosroshahi, et al., 2010, Arthritis Rheum. [epub ahead of print] PMID: 20191576; Keogh, et al., 2005, Arthritis Rheum. 52:262-8).

Unequivocal proof that a human disease syndrome is autoimmune is difficult to establish. Despite awareness for >50 years that many syndromes have an autoimmune basis (e.g., SLE), the pathogenic mechanisms of most autoimmune diseases have still not been completely described. (Marrack, et al., 2001, Nat Immunol 7:899-905; Lipsky, 2001, Nat Immunol 2:764-766). Nonetheless, the existence of an autoimmune disease is generally acknowledged when: 1.) The presence or extent of autoimmunity is clearly abnormal, including increased prevalence or concentrations of pathogenic self-reactive immunoglobulins or T-cells; 2.) The autoimmunity is directed against antigens within the diseased organ(s); 3.) There is evidence of autoimmune-mediated pathogenesis within the target organ (e.g., immune complexes or fixed complement); and 4.) Features of the autoimmune response are correlated with disease manifestations. (Duncan, 2010, Am J Respir Crit Care Med 181(1):4-5; Elkon, et al., 2008, Nat Clin Pract Rheumatol 4:491-498; Ermann, et al., 2001, Nat Immunol 2:759-761; Marrack, et al., 2001, Nat Immunol 7:899-905; Lipsky, 2001, Nat Immunol 2:764-766).

### 2.2 Lung Diseases

Emphysema and COPD are intractable problems that account for enormous world-wide morbidity and mortality (Lopez, et al., 2006, Lancet 367:1747-1757; Mannino, 2002, Chest 121:121S-6S). In addition to disability and early death attributable directly to these lung diseases, emphysema has been closely linked by epidemiologic studies to important systemic abnormalities, (Chatila, et al., 2008, Proceedings of the American Thoracic Society 5:549-555; Agusti, 2005, Proceedings of the American Thoracic Society 2:367-370; Decramer M, et al., 2008, COPD 5:235-256) including vasculopathies (McAllister, et al., 2007, Am J Resp Crit Care Med 176:1208-1214; Sabit, et al. 2007, Am J Respir Crit Care Med 175:1259-1265), osteoporosis (Sabit, et al., 2007, Am J Respir Crit Care Med 175:1259-1265) (de Vries, et al., 2005, Eur Respir J 25:879-884; Bolton, et al., 2004, Am J Resp Crit Care Med 170:1286-1293; Bon, et al., 2011, Am J Respir Crit Care Med 183:885-90), and lung cancer (Wilson et al., 2008, Am J Resp Crit Care Med 178:738-744).

Idiopathic pulmonary fibrosis (IPF) is a chronic, morbid, fibroproliferative lung disease that afflicts approximately 40,000 patients in the U.S. annually (American Thoracic Society, 2000, Am J Respir Crit Care Med 161: 646-664). IPF typically manifests with inexorably progressive pulmonary restriction and gas exchange abnormalities. Despite extensive study, the etiology of IPF remains enigmatic. Consequently, rational selection of therapies that specifically target the causal pathogenic process has not yet been possible. No medical intervention has proven efficacy and, while the disease course is both variable and unpredictable, the median survival of IPF patients is approximately 3 years or less after diagnosis (American Thoracic Society, 2000, Am J Respir Crit Care Med 161, 646-664).

Nevertheless, numerous studies of patient-derived clinical specimens have shown that abnormalities of adaptive immunity are common in IPF (Campbell, et al., 1985, Thorax. 40, 405-11; Marchal-Somme, et al., 2006, J Immunol. 176, 5735-5739; Zuo, et al., 2002, Proc Natl Mad Sci., USA 99 6292-97; Aglio, et al., 1988, Respiration 54, 36-41; Dobashi, et al., 2000, Lung 178, 171-9; Grigolo, et al., 1998, Clin Exp Immunol 114, 339-46; Yang, et al., 2002, Clin Exp Immunol 128, 169-74; Takahashi, et al., 2007, Respirology 12, 642-653; Wallace, et al., 2001, J Pathol 195, 251-6; Magro, et al., 2006, Hum Immunol 67, 284-297; Ogushi, et al., 2001, J Med Invest 48, 181-9; Kurosu, et al., 2008, J Immunol 181, 756-767; Feghali-Bostwick, et al., 2007, J Immunol 179, 2592-9; Gonzalez-Grwonow, et al., 2006, Cancer Res 66, 11424-11431; Feghali-Bostwick, et al., 2008, Am J Resp Critical Care Med 177, 156-163; Mayada, et al., 2009, Circulation 120, 2012-2024; Erickson, et al. 1979, Mayo Clin Proc. 54, 714-720; Prohászka, 2007, Adv Exp Med Biol 594, 159-66). B-cell follicular-like aggregates are evident in IPF lungs (Campbell, et al., 1985, Thorax 40, 405-11; Marchal-Somme, et al., 2006, J Immunol 176, 5735-5739), as well as over-expressions of immunoglobulin genes (Zuo, et al., 2002, Proc Natl Acad Sci., USA, 99, 6292-97). Antigen-antibody (immune) complexes are present in sera and bronchoalveolar lavage (BAL) of IPF subjects (Aglio, et al., 1988, Respiration 54, 36-41; Dobashi, et al., 2000, Lung 178, 171-9), and nearly all have circulating autoantibodies with diverse specificities (Dobashi, et al., 2000, Lung 178, 171-9; Grigolo, et al., 1998, Clin Exp Immunol 114, 339-46; Yang, et al., 2002, Clin Exp Immunol 128, 169-74; Takahashi, et al., 2007, Respirology 12, 642-653; Wallace, et al., 2001, J Pathol 195, 251-6; Magro, et al., 2006, Hum Immunol 67, 284-297; Ogushi, et al., 2001, J Med Invest 48, 181-9; Kurosu, et al., 2008, J Immunol 181, 756-767; Feghali-Bostwick, et al., 2007, J Immunol 179, 2592-9). The production of IgG autoantibodies against protein self-epitopes is dependent on the provision of facultative help by CD4 T-cells that have shared specificity for those antigens (Parker, 2006, Annu Rev. Immunol 11, 331-340).

CD4 T-cells of IPF patients are abnormally activated in situ, exhibit antigen-driven olicoclonal expansions, have highly altered end-differentiation phenotypes with impaired Treg activity and enhanced, disease-relevant effector functions, and are uniquely reactive to autologous intrapulmonary antigen(s) (Feghali-Bostwick, et al., 2007, J Immunol, 179, 2592-9; Gonzalez-Grwonow, et al., 2006, Cancer Res. 66, 11424-11431; Feghali-Bostwick, et al., 2008, Am J Resp Critical Care Med 177, 156-163; Mayada, et al., 2009, Circulation, 120, 2012-2024; Erickson, et al. 1979, Mayo Clin Proc 54, 714-720).

Features of IPF are generally congruent with prototypical manifestations of autoimmune disorders, including familial predilections (Rosas, et al., 2007, Am J Resp Crit Care Med 176, 698-705), associations with over-representations of specific HLA alleles (Xue, et al., 2011, PLoS One 6:e14715), frequent failure of glucocorticoid regimens to alter the disease natural history (American Thoracic Society, 2000, Am J Respir Crit Care Med 161, 646-664; Collard, et al., 2007, Am J Respir Crit Care Med 176, 636-643), ongoing target organ injuries that persist despite removal of putative precipitating factors (e.g., smoking cessation, changes in environment), and sudden disease exacerbations that occur in the absence of other obvious processes (e.g., infections; Collard, et al., 2007, Am J Respir Crit Care Med 176, 636-643).

### 2.3 Heat Shock Proteins

Hsp are chaperone molecules that transport intracellular proteins to the endoplasmic reticulum (ER), wherein those complexed proteins are processed, and the constituent peptides are presented by HLA of antigen-presenting cells (APC) for surveillance by T-cells (Prohaska, 2007, Adv Exp Med Biol 594: 159-166; Tahiri, et al., 2008, Hepatology 47: 937-948; Zlacka, et al., 2006, J Autoimmune 27: 81-88; Abulafia-Lapid, et al., 2003, J Autoimmune 20: 313-321). Hsp-peptide complexes are also present in the extracellular milieu, where they bind to specific APC receptors, are internalized, transported to the ER, and again, incorporated onto nascent HLA molecules. Hence, Hsp are intimately associated with antigen transport, processing, and presentation to the T-cells that initiate and drive adaptive immune responses, ultimately including production of self-avid IgG (Parker, 2006, Annu Rev Immunol 11:331-340).

### 3. SUMMARY OF THE INVENTION

The present invention relates to methods of diagnosing and/or determining a prognosis for various diseases, including autoimmune diseases and lung diseases such as idiopathic pulmonary fibrosis (“IPF”), chronic obstructive pulmonary disease (“COPD”), and emphysema as well as osteoporosis, comprising detecting the presence of autoantibodies specific for heat shock proteins in a subject. It is based, at least in part, on the discovery of autoantibodies directed to heat shock protein 70 (“hsp70”) and/or glucose regulated protein 78 (“GRP78”) in human patients suffering from these diseases, in some subjects associated with the DRB1*15 genotype. In the case of lung diseases, the presence of such antibodies was found to correlate with shortened survival and greater impairment of pulmonary function. An autoimmune T cell response to Hsp70 protein was also detected in subjects suffering from chronic lung disease.

In another, related aspect, the present invention relates to methods of diagnosing and/or determining a prognosis for IPF in a subject comprising determining that the subject carries a single nucleotide polymorphism (“SNP”) of hsp70. This aspect is based, at least in part, on the discovery that IPF patients having the rs1061581 allele of HSP70A1B were found to exhibit longer survival than subjects not having this allele.

The information gained regarding diagnosis and/or prognosis may then be utilized by a health care professional to provide a recommendation regarding care options.

### 4. BRIEF DESCRIPTION OF THE FIGURES

FIG. 1A-C. Circulating anti-Hsp70 autoantibodies, and Hsp70 expression in lung specimens. A.) Prevalences of circulating anti-Hsp70 autoantibodies in healthy normal controls and IPF patients are depicted. Respective subject numbers (n) are denoted within columns. B.) (Row 1): Immunoblots showing representative Hsp70 autoantibody detections in IPF and control subjects; (Row 2): detection of Hsp70 antigen by immunoblot in IPF and control lung extracts (n=6 each); and (Row 3): detection of Hsp70 antigen by immunoblot in IPF and control bronchoalveolar lavage fluid (BALE) (n=5 each). C.) Comparative densitometry measurements (arbitrary units) of Hsp70 antigen immunoblots of lung extracts and BALF. Alpha (p) values for intergroup comparisons are denoted above brackets.

FIG. 2A-F. In situ localization of Hsp70 and IgG immune complexes by immunohistochemistry in IPF lung explants. A.) Hsp70 was highly expressed in distal airway epithelium, and to lesser extents in pneumocytes, macrophages, and occasional endothelial cells (10×). B.) More detailed image of Hsp70 expression in IPF lungs (20×). C.) Isotype control for Hsp70 staining (20×). D.) IgG immune complexes were prevalent in IPF lungs (10×). E.) Higher power view showing diffuse, dense distribution of IgG-antigen complexes in IPF lungs (40×). F.) Isotype control for the immune complex staining (20×).

FIG. 3A-B. Clinical associations of anti-Hsp70 autoreactivity in IPF patients. A.) Decrements of forced vital capacities, as percentages of predicted values (FVC % p), were greater among those nine (9) IPF subjects who had anti-Hsp70 autoantibodies (anti-Hsp70 Pos) and subsequent pulmonary function tests (PFTs), compared to 41 IPF subjects with anti-Hsp70 autoantibodies (anti-Hsp70 Neg) and later PFTs, and there was a trend for decrements of single breath diffusing capacities for carbon monoxide (DLCO), as percent predicted values (DLCO % p), These PFTs were performed 5.9+1.4 and 6.4+0.4 months after the plasma acquisitions of the anti-Hsp70 Neg and anti-Hsp70 Pos subjects, respectively. B.) Survival in the year following Hsp70 antibody measures was significantly decreased among IPF patients with these autoantibodies, compared to those patients who did not have this immune response. Cross-hatches denote censored events. Numbers in parentheses at the terminus of the survival curves denote subjects censored at the conclusion of the observation period (one year).

FIG. 4A-B. HLA-DRB1*15 in IPF and normal subjects. A.) Prevalences of HLA-DRB1*15 in the respective IPF cohorts (left panel) and the aggregate IPF and normal control populations (right panel) are depicted. UPMC Tx denotes the discovery cohort, consisting of patients who had lung transplantation at this site. NIH denotes the National Institute of Heath cohort, consisting of ambulatory IPF patients. UPMC OP denotes those IPF patients who were ambulatory (Out-Patients) at the time their plasma for the autoantibody studies was acquired. Thirteen (13) of these subjects subsequently had lung transplantations and their HLA data had already been compiled as subjects within the discovery cohort (UPMC Tx). Normal controls included subjects from UPMC and the NIH (see text). Respective subject numbers are denoted within each column. B.) HLA-DRB1*15 was more prevalent among IPF patients who had anti-Hsp70 autoantibodies (Anti-Hsp70 Pos) than among those patients who did not have this immune response.

FIG. 5. Two-dimensional (2-D) gel electrophoresis of cell lysate proteins immunoprecipitated by pooled IPF patient IgG. Numbered circles denote distinct cell lysate proteins resolved by this technique and visualized by Coomassie stain. Mass spectroscopy of selected picked proteins resulted in high confidence identifications of Hsp70 isoforms (spots 2-4) and Hsp27 (spot 17), among others.

FIG. 6. Single breath diffusing capacities for carbon monoxide (DLCO), as percentages of predicted values (DLCO % p), were decreased in the IPF patients with HLA-DRB1*15, compared to those who did not have this allele, among each individual study cohort. UPMC Tx denotes the discovery cohort of lung transplant recipients at the University of Pittsburgh Medical Center; UPMC OP denotes a replication cohort of ambulatory IPF patients from that institution (initially recruited for autoantibody studies); and NIH denotes a replication cohort of subjects from the National Institute of Health.

FIG. 7A-FI. Initial T-cell Antigen Characterizations. A.) Proliferation of hilar lymph node cells were determined in cultures supplemented with protein fractionations of autologous lung extracts that had been separated by isoelectric focusing (IEF). In all but one case (denoted by dashed lines) the most acidic protein fractions induced the greatest proliferation of autologous hilar lymph node cultures. B.) Replicate studies of additional specimens using ion exchange chromatography to separate proteins, and BrdU incorporation to specifically measure CD4 T-cell proliferation, resulted in analogous findings. C.) Separation of the lowest pI IEF fractions by 50 kDa cut-off centrifugation filtration indicated the greatest antigenicity was within the larger protein fraction. The limited amount of protein in one specimen precluded further study. D.) Indirect fluorescence assays for detection of human IgG autoantibodies with specificities for HEp-2 cell antigens. Seven of 10 IPF plasma specimens were positive, compared to one of 5 specimens from normal subjects. D-I) Diverse patterns of immunofluorescence, as seen in these IPF subjects, are consistent with the presence of numerous autoantibodies with diverse antigen specificities: (aD.) nucleolar and diffuse, (bE.) and (cF.) diffuse, and (dG.) rimmed. Specimens from IPF subjects depicted here in panels bE and dG had anti-HSP70 autoantibodies, and the former also had autoantibodies to GRP78. Normal control plasma specimens are depicted in panels EH and FI.

FIG. 8A-C. Autoantibodies and Antigen in Healthy Controls and IPF Patients. A.) Prevalences of circulating anti-HSP70 and anti-GRP78 IgG autoantibodies in healthy normal controls and IPF patients are depicted. Respective subject numbers (n) are denoted within columns. The number of IPF subjects tested for anti-GRP78 autoantibodies is smaller due to prior depletion of plasma from three subjects. B.) Concordances between anti-HSP70 and anti-GRP78 autoreactivity. Numbers within quadrants denote the prevalence (%) of the respective autoantibodies. A minority of the IPF subjects had concurrent autoantibody responses to both heat shock proteins. C.) Immunoblots of HSP70 and GRP78 in pulmonary specimens from randomly chosen lung explants. Both heat shock proteins were present in water soluble lung extracts and bronchoalveolar lavage (BAL) from all IPF patients, but less frequently and usually in lesser amounts in equivalent specimens from normal lungs.

FIG. 9A-D. T-cell Functional Assays in IPF Specimens. A.) Addition of HSP70 resulted in greater proliferation of peripheral CD4 T-cells, ascertained by BrdU incorporation, than in controls (Cnt) with no added protein, or in concurrent cultures supplemented with either GRP78 or tetanus toxid (TdT) (n=18). Pc denotes alpha values corrected for multiple comparisons by Bonferroni. Horizontal bars denote mean values. Proliferation within GRP78 and TdT supplemented cultures did not significantly differ from those of controls. B.) Specific index (SI) of proliferation (experimental values with added antigen minus those of baseline controls). C.) IPF CD4 T-cell IL-4 production was also more enhanced by HSP70 than by the other antigens (n=15). D.) SI of CD4 T-cell IL-4 production.

FIG. 10A-D. Clinical Correlates of Heat Shock Protein Autoreactivities. A.) Surviving IPF subjects with anti-HSP70 autoantibodies (Antibody Pos) (n=11) had greater subsequent decrements of forced vital capacities, as % of predicted values (FVC % p), and a tendency for greater decrements of percent predicted diffusing capacity for carbon monoxide (DLCO % p), compared to the subjects without this autoantibody (Antibody Neg, n=52). Pulmonary function determinations were made ˜6 months after the plasma sample acquisitions. B.) There were no apparent associations between anti-GRP78 humoral autoreactivity and subsequent changes of pulmonary function. C.) IPF patients with anti-HSP70 autoantibodies had decreased survival. Cross hatches denote censored events, and numbers in parentheses denote subjects censored at the end of observation. D.) The presence or absence of anti-GRP78 autoantibodies (dashed and solid lines, respectively) did not associate with survival differences.

FIG. 11A-L. Lung Immunohistochemistry. Columns from left to right depict, respectively, expression of HSP, 70 (A, E, I), IgG immune complexes (B, F, fixed complement (C3) (C, G, K), and isotype controls (D, H, L). Rows, from top to bottom respectively, depict end-stage IPF lungs explained during therapeutic pulmonary transplantations (Explant) (n=6) (A, B, C, D), warm autopsies from patients who died during acute exacerbations (AE) of TPF (n=3) (E, F, G, H), and normal lungs harvested during multi-organ retrievals, but not used in therapeutic transplantations (n=6) (I, J, K, L).

FIG. 12A-E. Anti-HSP70 Autoreactivity in COPD. A.) Prevalence of anti-HSP70 autoantibodies in COPD patients, stratified on the basis of their disease severity (GOLD Stage) (46). Numbers in columns denote subject n within the respective GOLD cohorts. B.) Proliferation of CD4 T-cells from COPD patients (n=22) was not significantly increased by addition of HSP70, in contrast to TdT supplemented cultures. Pc=alpha value corrected for multiple comparison by Bonferroni. Horizontal bars denote population mean values. C.) and D.) IL-4 production of COPD CD4 T-cells was not increased by HSP70. Because immune responses of COPD may be TH1 biased (43,48), the production of IFN-□ was also determined in these subjects (n=21), but production of this cytokine was similarly not increased in cultures supplemented with HSP70. E.) Changes of pulmonary function, measured two years after the initial plasma specimen was obtained, were not appreciably different between those COPD subjects who had anti-HSP70 autoantibodies (Antibody Pos) vs, those who did not have these autoantibodies (Antibody Neg). FEV1% p=forced expiratory volume in the first second of expiration, as a percentage of predicted values. Only data for GOLD 2-4 subjects is depicted here, given the low prevalence of anti-HSP70 autoantibodies in GOLD 1 patients (FIG. 12A).

FIG. 13. Two-dimensional (2-D) gel electrophoresis of K652 cell lysate proteins immuno-precipitated by pooled IPF patient IgG. Numbered circles denote distinct cell lysate proteins resolved by this technique and visualized by Coomassie stain. Mass spectroscopy of these proteins resulted in high confidence identifications of GRP78 (spot #1), HSP70 isoforms (spots #2-4), and albumin (spot #5).

FIG. 14. Association of anti-Hsp70 autoreactivity and DRB1*1501 expression. This HLA allele was over-represented among IPF patients with autoantibodies to Hsp70.

FIG. 15. Anti-Hsp70 autoantibodies and survival. IPF patients with autoantibodies to Hsp70 had much worse one year survival, compared to the cohort who did not have this autoimmune response.

FIG. 16. Two-year survival of IPF patients with the GG genotype at rs1061581 was significantly greater than that of subjects with the other two genotypes at this locus. Subjects include both U. Pittsburgh discovery and NIH replication cohorts. Hazard ratio (HR) and 95% confidence intervals (CI) for the GG genotype are denoted.

FIG. 17. Production of HSP70 in lysates of primary human pulmonary fibroblasts was determined in basal cultures and compared to production of paired, autologous fibroblasts harvested 12 hours after heat shock for 30 minutes. Both specimens from GG subjects showed stress caused decreased HSP70 production, unlike cells from subjects with the other two genotypes. HSP70 measures are based on densitometry of immunoblots, standardized for total protein content. Each data point represents cells lines from genotyped IPF subjects. Data are depicted as means±SE.

FIG. 18. The prevalence of anti-HSP70 autoantibodies among IPF patients with the AA genotype of HSP70-hom was less than ½ that of subjects with the other SNP at this locus. OR=odds ratio.

FIG. 19A-B. IgG-antigen complexes (A) and fixed complement (B) are specific features of antibody-mediated pathogenesis and are common in end-stage COPD lungs.

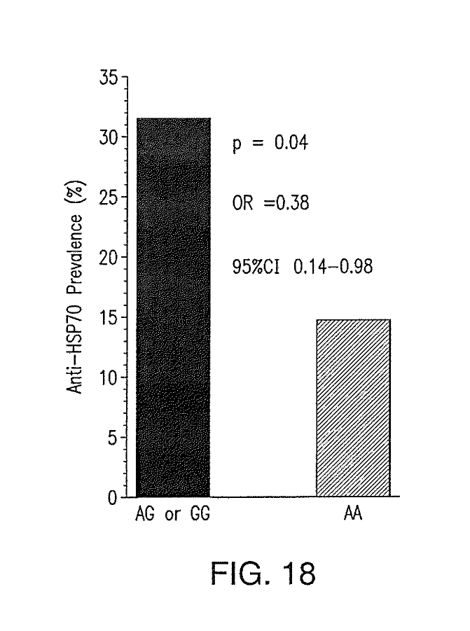

FIG. 20A-B. (A) Heterogeneous autoantibodies with diverse specificities (varied fluorescent patters) were seen among COPD subjects with indirect immunofluorescent assays (IIF). B) The prevalence of autoantibodies was much greater in COPD patients than in age and gender matched controls with no smoking histories (Cnt), or age and gender matched subjects with normal spirometry and extensive smoking exposures (SC). Numbers within columns denote group n.

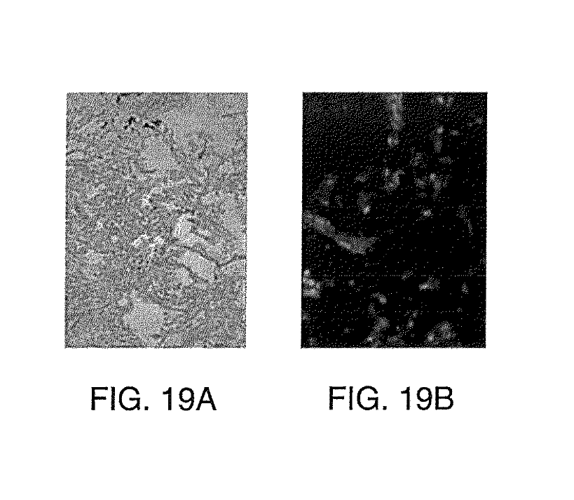

FIG. 21. (Right) IgG within individual COPD samples were adsorbed to protein A and used to immunoprecipitate radiolabeled K562 cell lysate proteins. Each lane represents an individual COPD specimen. Bands denote cellular proteins (putative autoantigens) captured by COPD IgG. kD is a molecular weight standard. Std is a standard panel of known antigens of conventional autoimmune diseases.

FIG. 22. (left) 2-D gel resolution of cell lysate proteins isolated by immunoprecipitation (IP) using pooled COPD autoantibodies. Circled spots (red) indicate individual proteins (putative autoantigens).

FIG. 23. Anti-GRP78 autoantibodies are more prevalent in patients with advanced COPD compared to age and gender matched, nonsmoking controls (Cnt), or age and gender matched subjects with normal spirometry and extensive smoking exposures (SC). G denotes GOLD stage31 and numbers within columns denote group n.

FIG. 24. GRP78 immunoblot of 6 COPD BAL (36 μg BALF protein/lane)

FIG. 25. GRP78 is evident in airway and alveolar epithelium, and macrophages of COPD lungs. Right panel is an isotype control. (anti-GRP mAb is from Abcam)

FIG. 26A-B (A). Specific proliferation (antigen stimulated minus control unstimulated) BrdU incorporation among CD4 T-cells from blood of COPD patients in 4 day in vitro cultures with 1 μg/ml putative antigens. Pilot study did not show consistent effects of antigen titrations from 0.1-to-10 μg/ml. (B) Specific production of CD4 T-cell intracellular IFN-γ in 2 day cultures (GolgiStop™ added 5 hours before harvest). Elastin=elastin split products; TdT=tetanus toxoid; kexin is a recombinant cell wall component of Pneumocystis (IFN-γ studies pending). P values are for paired comparisons between control and antigen-stimulated values. Numbers in symbols denote group n.

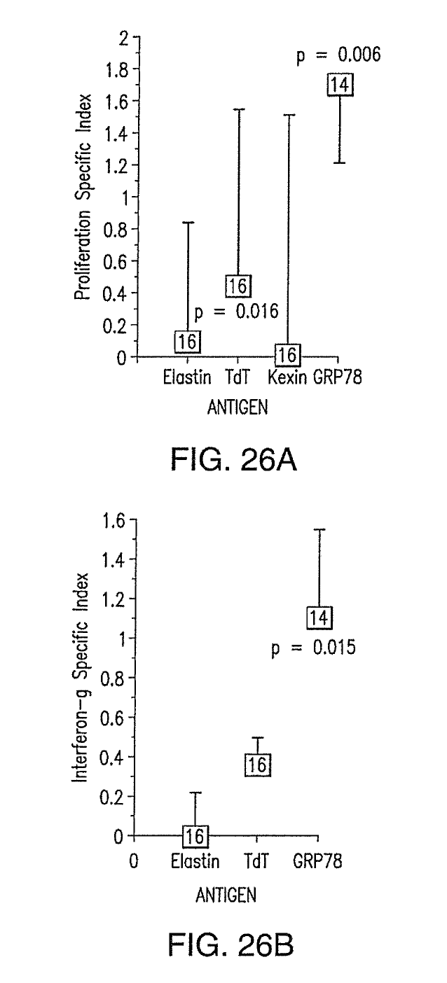

FIG. 27A-B. Both the prevalence (A) and magnitude (B) of emphysema was greater in study subjects who had anti-GRP78 autoantibodies.

FIG. 28A-B. Emphysema prevalence (A) and magnitude (B) is greatest in SC.

FIG. 29A-B. Anti-GRP78 autoantibodies versus emphysema prevalence (A) and magnitude (B) in female SC.

FIG. 30A-B. The presence of anti-GRP78 autoantibodies was associated with many abnormal bone mineral density measures. This autoantibody association was greatest among the cohort with COPD (B; A is all subjects)).

### 5. DETAILED DESCRIPTION OF THE INVENTION

For clarity and not by way of limitation, the detailed description of the invention is divided into the following subsections:

(i) Hsp70 autoantigens

(ii) Hsp70 SNP;

(iii) diseases associated with Hsp70 autoantibodies; and

(iv) clinical methods.

### 5.1 HSP 70 Autoantigens

In various embodiments of the invention, autoantigens are members of the hsp70 protein family.

The human hsp70 family has at least eight members (Daugaard et al., 2007, FEBS LETTERS 581:3702-3710), as follows:

Hsp70-1a, alternatively referred to as Hsp70, Hsp72, or Hsp70-1, accession number NM_{—}005345;

Hsp70-1b, alternatively referred to as Hsp70, Hsp72, or Hsp70-1, which is 99% homologous to Hsp70-1a, accession number NM_{—}005346;

Hsp70-1t, alternatively referred to as Hsp70-hom, which is 1% homologous to Hsp70-1a, accession number NM_{—}005527;

Hsp70-2, alternatively referred to as Hsp70-3 or HspA2, which is 84% homologous to Hsp70-1a, accession number NM_{—}021979;

Hsp70-5, alternatively referred to as Bip or Grp78, which is 64% homologous to Hsp70-1a, accession number NM_{—}005347;

Hsp70-6, alternatively referred to as Hsp70B, which is 85% homologous to Hsp70-1a, accession number NM_{—}002155;

Hsc70, alternatively referred to as Hsp70-8 or Hsp73, which is 86% homologous to Hsp70-1a, accession number NM_{—}006597; and

Hsp70-9, alternatively referred to as Grp75, mtHsp75 or Mortalin, which is 52% homologous to Hsp70-1a, accession number NM_{—}004134.

Non-human homologues of the above-listed hsp70 family members are known in the art, and are encompassed by the invention where the subject of interest is a non-human.

The existence of an autoantigen in a subject may be determined by detecting, in the subject, an antibody or antibody fragment that specifically binds to said autoantigen (referred to as an “autoantibody”). Methods of detecting the presence of antibodies directed at antigens of interest in a subject are well known in the art, and include, but are not limited to, techniques in which the target antigen (or a portion thereof) is immobilized, exposed to antibody from a patient sample, and antibodies bound to target antigen are detected, for example by a detectably labeled secondary antibody or labeled antigen or labeled antigen fragment. Specific techniques for detecting and/or measuring antibodies in a patient sample include but are not limited to enzyme-linked immunosorbent assay (“ELISA”) and detection of antibody by biosensor array. Accordingly, the present invention provides for detecting and/or measuring autoantibodies directed to a member of the Hsp70 family of proteins.

Further, the existence of an autoantigen in a subject may be determined by detecting, in the subject, the presence of T lymphocytes that can be activated by said autoantigen. Methods of detecting such activated T cells are well known in the art, and include, but are not limited to, assays in which peripheral blood mononuclear cells collected from the subject are exposed to the autoantigen, and then proliferation of said cells is measured (for example, by incorporation of radioisotope into DNA), where proliferation is indicative of the presence of activated T cells. The presence of T cells that can be activated autoantigen is referred to herein as T cell autoimmunity.

### 5.2 HSP70 SNP

As described more fully in example section 9 below, patients suffering from IPF having the rs1061581 allele of HSP70A1B were observed to exhibit longer survival than subjects having other known alleles of Hsp70 (for example the rs104361 allele of HSP70A1A or the and rs2227956 of HSP70A1L (HSP70-hom). The presence of the rs1061581 allele may be determined by any method known in the art. In a specific non-limiting embodiment, the presence of the rs1061581 allele may be determined by PCR-based restriction fragment length polymorphism (RFLP) analysis. For example, a 1117 bp DNA fragment may be amplified by PCR using primers 5′-CAT CGA CTT CTA CAC GTC CA-3′ (SEQ ID NO:1) and 5′-CAA AGT CCT TGA GTC CCA AC-3′ (SEQ ID NO:2) in the presence of the MasterAmp PCR buffer G (Epicentre, Madison, Wis.). Then, the PCR products may be digested with Pst I which differentially digest the rs1061581G allele to generate two fragments with 184 bp and 933 bp in size. Genotypes may then be determined based on band patterns of an agarose gel electrophoresis analysis of the digested PCR products. The rs1043618 of HSP70A1A and rs2227956 of HSP70A1L (HSP70-hom) may be analyzed using Taqman SNP printer/probe set C_{—}11917510_{—}10 and C_{—}25630755_{—}10, respectively, and 7900 HT DNA analyzer (Applied Biosystems, Foster City, Calif.).

### 5.3 Diseases Associated with Hsp 70 Autoantibodies

According to the present invention, diseases associated with Hsp70 autoantibodies include autoimmune diseases and chronic lung diseases such as IPF, COPD and emphysema, as well as osteoporosis.

### 5.4 Clinical Methods

The present invention provides for methods of diagnosing and/or assessing the prognosis of a disease, in a subject, selected from the group consisting of a chronic lung disease such as IPF, COPD and emphysema and osteoporosis, comprising determining whether the subject has developed an autoimmune response to a Hsp70 autoantigen, as described above. An autoimmune response may be indicated by the presence of autoantibodies and/or T cells that may be activated by an Hsp70 autoantigen. The presence of such an autoimmune response is supportive of the diagnosis of chronic lung disease such as IPF, COPD and emphysema or osteoporosis, and, where present, is indicative that the subjects condition is clinically more severe than that of a subject that does not exhibit an autoimmune response to Hsp70 autoantigen. For chronic lung diseases, this increased severity may be manifested by more severely impaired lung function, for example as measured by pulmonary function tests such as forced vital capacity, and/or shortened survival.

In the case of chronic lung disease, further support to a diagnosis of IPF, COPD or emphysema may be provided by determining that the subject has the DRB1*15 genotype.

In further nonlimiting embodiments of the invention, determining that a subject suffering from chronic lung disease has the DRB1*11 genotype makes it less likely that the subject has autoantibodies directed toward Hsp70 autoantigens. In specific non-limiting embodiments, if a subject is determined to have the DRB1*11 genotype, this is consistent with (but not determinative of) a better prognosis in terms of survival and pulmonary function.

In further nonlimiting embodiments of the invention, determining that a subject suffering from IPF has the rs106581 Hsp allele is consistent with (but not determinative of) a better prognosis in terms of survival and pulmonary function.

In the case of a chronic lung disease, where an autoimmune response to a Hsp70 autoantigen has been detected in a subject, in non-limiting embodiments a health care provider may recommend an interventional step, for example, but not limited to, lung transplant, lung biopsy (transplant recipient only), closer observation and serial PFT testing, augmentation or addition or substitution of immunosuppressive medications.

In the case of osteoporosis, where an autoimmune response to a Hsp70 autoantigen has been detected in a subject, in non-limiting embodiments a health care provider may recommend an interventional step, for example, but not limited to, pharmacotherapy with a bisphosphonate drug, raloxifene, calcitonin, teriparatide, or hormone therapy.

Accordingly, in one set of embodiments, the present invention provides for a method of diagnosing a chronic lung disease, selected from the group consisting of idiopathic pulmonary fibrosis, chronic obstructive pulmonary disease, and emphysema, in a subject, comprising determining whether the subject has developed an autoimmune response to a Hsp70 autoantigen, where the presence of the autoimmune response supports the diagnosis of the chronic lung disease. The determination of whether the subject has developed the autoimmune response may comprise determining whether a sample from the subject contains antibodies specific for the Hsp70 autoantigen and/or whether a sample from the subject contains T cells that are capable of being activated by the Hsp70 autoantigen. In specific, non-limiting examples, the Hsp70 autoantigen may be Hsp70-1a or Grp78. In specific non-limiting embodiments, the method may further comprise determining whether the subject exhibits the DRB1*15 genotype, where the presence of said genotype further supports the diagnosis of the chronic lung disease.

In another set of non-limiting embodiments, the present invention provides for a method of determining the prognosis of a subject suffering from a chronic lung disease, selected from the group consisting of idiopathic pulmonary fibrosis, chronic obstructive pulmonary disease, and emphysema, comprising determining whether the subject has developed an autoimmune response to a Hsp70 autoantigen, where the presence of the autoimmune response indicates a poorer prognosis. The determination of whether the subject has developed the autoimmune response may comprise determining whether a sample from the subject contains antibodies specific for the Hsp70 autoantigen and/or whether a sample from the subject contains T cells that are capable of being activated by the Hsp70 autoantigen. In specific, non-limiting examples, the Hsp70 autoantigen may be Hsp70-1a or Grp78.

In another set of non-limiting embodiments, the present invention provides for a method of treating a subject suffering from a chronic lung disease, selected from the group consisting of idiopathic pulmonary fibrosis, chronic obstructive pulmonary disease, and emphysema, comprising determining whether the subject has developed an autoimmune response to a Hsp70 autoantigen, and, where the autoimmune response is present, recommending a further interventional step selected from the group consisting of lung transplant, lung biopsy (transplant recipient only), closer observation and serial PFT testing, and augmentation or addition or substitution of immunosuppressive medication. The determination of whether the subject has developed the autoimmune response may comprise determining whether a sample from the subject contains antibodies specific for the Hsp70 autoantigen and/or whether a sample from the subject contains T cells that are capable of being activated by the Hsp70 autoantigen. In specific, non-limiting examples, the Hsp70 autoantigen may be Hsp70-1a or Grp78.

In another non-limiting embodiment, the present invention provides for a method of diagnosing osteoporosis in a subject, comprising determining whether the subject has developed an autoimmune response to a Hsp70 autoantigen, where the presence of the autoimmune response supports the diagnosis of osteoporosis. The determination of whether the subject has developed the autoimmune response may comprise determining whether a sample from the subject contains antibodies specific for the Hsp70 autoantigen and/or whether a sample from the subject contains T cells that are capable of being activated by the Hsp70 autoantigen. In specific, non-limiting examples, the Hsp70 autoantigen may be Hsp70-1a or Grp78.

In another non-limiting embodiment, the present invention provides for a method of treating a subject suffering from osteoporosis, comprising determining whether the subject has developed an autoimmune response to a Hsp70 autoantigen, and, where the autoimmune response is present, recommending treatment with an agent selected from the group consisting of. bisphosphonate drug, raloxifene, calcitonin, teriparatide, or hormone therapy. The determination of whether the subject has developed the autoimmune response may comprise determining whether a sample from the subject contains antibodies specific for the Hsp70 autoantigen and/or whether a sample from the subject contains T cells that are capable of being activated by the Hsp70 autoantigen. In specific, non-limiting examples, the Hsp70 autoantigen may be Hsp70-1a or Grp78.

In another non-limiting embodiment, the present invention provides for a method of determining the prognosis of a subject suffering from a chronic lung disease, selected from the group consisting of idiopathic pulmonary fibrosis, chronic obstructive pulmonary disease, and emphysema, comprising determining whether the subject has the DRβ1*11 genotype, where the presence of said genotype indicates a better prognosis.

In another non-limiting embodiment, the present invention provides for a method of determining the prognosis of a subject suffering from idiopathic pulmonary fibrosis, comprising determining whether the subject has the rs1061581 allele, where the presence of said allele indicates a better prognosis.

The present invention provides for kits that may be used in any of the foregoing methods, which may comprise, for example, materials to be used in an ELISA assay, for example a sample of Hsp70 target antigen or antigens and optionally a detectably labeled secondary antibody for detecting antibody bound to said target antigen, and/or a sample of detectably labeled target antigen. A kit may also or alternatively comprise Hsp70 target antigen or antigens to be used in a T cell activation assay, for example with a label that detects cell proliferation, such as BrdU. A kit may also or alternatively comprise primers for detection of the rs1061581 allele, and/or DRB1*15, and/or DRB1*11.

### 6. Example 1

### Heat Shock Protein 70 Autoreactivity in Idiopathic Pulmonary Fibrosis

### 6.1. Materials and Methods

Subjects: Plasma specimens for autoantibody studies were obtained by centrifugation of anti-coagulated peripheral blood from consecutive ambulatory IPF patients at the Simmons Center for Interstitial Lung Disease at UPMC. At least twelve (12) months of subsequent observation from the time of specimen acquisitions was available on all of these subjects who did not succumb or undergo lung transplantation during this interval. Control plasma for autoantibody assays was procured from among normal volunteers recruited from hospital personnel by solicitation. All plasma specimens were stored at −80° C. until batch processing and use in these assays.

The initial (discovery) IPF cohort for HLA Class II allele characterizations consisted of consecutive patients with end-stage pulmonary disease who had molecular HLA allele determinations prior to their lung transplantations at UPMC.

The subsequent, prospective replication cohort for HLA allele assays was composed of IPF subjects for whom molecular HLA typing results and/or tissue specimens for HLA typing were available from the National Institute of Health (NIH), Inova Fairfax Hospital, and Stanford University Medical Center. IPF patients from the NIH were recruited during the course of other clinical studies (33), and were not lung transplantation recipients. IPF subjects from Inova and Stanford had undergone lung transplantations for end-stage pulmonary disease. In addition, the ambulatory UPMC IPF subjects who had determinations of autoantibodies (above) were also subsequently evaluated for the presence of HLA-DRB1*15, and used as another prospective HLA validation cohort.

HLA controls consisted of normal subjects who had prior molecular HLA typing in the course of other studies at UPMC (32) and the NIH (34).

Diagnoses were prospectively established in IPF subjects by expert, specialized pulmonary clinicians, blinded to these experimental studies, who analyzed all clinical information, including medical histories and physical exams, pulmonary function tests (PFTs), and laboratory studies that included serologic tests for conventional autoimmune syndromes, as well as expert rheumatology specialist evaluations, chest radiographs, and thoracic computerized tomography scans. All IPF study subjects fulfilled consensus diagnostic criteria (1). None had clinical evidence or a past history of connective tissue diseases, or drug toxicities or occupational/environmental exposures associated with interstitial lung disease. Extensive histological evaluations of IPF pulmonary explants were performed in all specimens removed during therapeutic transplantations by expert lung pathologists who were unaware of these studies. All lung explants from these subjects had histological confirmation of usual interstitial pneumonia or end-stage fibrotic disease.

Analyses are restricted to Caucasian IPF and control subjects because <5% of the initial IPF subjects were members of minority groups, and immunologic determinants, notably including HLA allele frequencies, can vary greatly among racial/ethnic subpopulations (35).

The study was approved by the respective Institutional Review Boards of all the participating medical centers.

Lung Explant Specimens: Methodologies for procurement and processing of these specimens have been detailed previously (14). In brief, surgically explanted lungs were obtained from IPF patients undergoing therapeutic pulmonary transplantations. Lung tissue (˜0.5-1 cm3) was dissected from the explants, embedded in optimal cutting temperature (OCT) media, frozen, and stored at −80° C.

Water soluble protein extracts of lung explants were also obtained by repeated freezing and thawing of additional tissue aliquots, as detailed elsewhere (14). These specimens have been previously shown to be a source of antigens for adaptive immune responses in IPF patients (14).

BALF was obtained from the explants by wedging sterile 5 mm plastic tubing in segmental bronchi of the middle lobe or lingula and infusing and withdrawing five successive 30 ml aliquots of PBS using a syringe. The BALF was centrifuged (400 g) and filtered (0.4 μm) to remove cells and particulates before storage at −80° C. Proteins in BALF were concentrated using 10 kDa centrifugation-size filters (Millipore, Bellerica, Mass.), and quantitated by bicinchoninic acid (BCA) assay (Thermo Scientific, Rockfort, Ill.).

Normal pulmonary specimens were similarly obtained from harvests of cadaveric lungs that were not used for transplantations.

No lung specimen used in these studies had evidence of infection by clinical features, gross examination, microbiologic cultures, or histological evaluations.

Autoantigen Discovery: IgG was isolated from pooled plasma of six IPF patients, already known to have autoantibodies on prior study (14), by adherence to protein A columns (HP SpinTrap, GE Healthcare, Piscataway, N.J.). After extensive washing, the bound antibodies were covalently cross-linked to the protein A, following the manufacturer's protocol. Otherwise identical “preadsorption” columns were prepared using IgG from normal control plasma specimens.

K562 cells in exponential growth phase were disrupted by sonication. K562 cell lysates were used because prior study showed they were a rich source of autoantigens for IPF patients (14), including a cryptic autoantigen(s) of ˜70-78 kDa that seemed associated with pulmonary function decrements (unpublished data). These cell lysates were initially preadsorbed, to eliminate nonspecific binding, by application to the normal IgG-protein A columns. The eluants of these preadsorbtions were then applied to the IPF patient IgG-protein A columns and extensively washed. The captured cell proteins (putative autoantigens) were then eluted by acidification, followed by pH neutralization, and concentrated by centrifugal size-filtration.

These immunoprecipitation preparations were electrophoresed by two dimension 10.5% sodium dodecyl sulfate polyacrylamide gel electrophoresis (SDS-PAGE). Gels were imaged by Typhoon TRIO (GE Healthcare) and analyzed by Image QuantTL software (GE Healthcare). Individual proteins were harvested by spot picking (Ettan Spot Picker, GE Healthcare), trypsin digested, and peptides sequences identified by matrix-assisted laser desorption/ionization tandom time of flight mass spectrometry (MALDI-TOF/TOF) (Applied Biosystems, Carlsbad, Calif.),

Autoantibody Assays: Circulating autoantibodies against specific proteins were detected in individual patient plasma by immunoblots. Recombinant proteins were purchased from Prospec (Rehovot, Israel), and 250 ng was added to each lane of running gels (NuPage 4-12% Bis-Tris, Invitrogen, Carlsbad, Calif.). After electrophoresis (90 minutes @130 V), the protein was transferred to 0.45 μm nitrocellulose membranes (Invitrogen). Gels were blocked with 5% dry milk in TTBS (50 mM Tris HCl [pH 7.4], 150 mM NaCl, 0.1% Tween 20). Individual lanes were separated by sectioning and incubated with subject sera (1:10 dilution in the blocking buffer) overnight at 4o. The membrane strips were extensively washed in TTBS, and then incubated for 1 hour at room temperature with 1:8000 dilutions of chicken anti-human IgG conjugated to horseradish peroxidase (HRP) (Thermo Scientific). After another extensive washing, the HRP was detected on radiographic film after addition of Super Signal West Pico Chemiluminescent Substrate (Thermo Scientific). The specificity of these immunoblots was confirmed by showing identical findings using monoclonal mouse anti-human Hsp70 autoantibody (United States Biological, Swampscott, Mass.) at a dilution of 1:1000 in lieu of IPF patient plasma (positive controls).

Detection of Hsp70 in BALF and Lung Extracts: Immunoblots were also used to detect Hsp70 in clinical specimens. Individual aliquots of lung extract (144 μg protein) or BALF (36 μg protein) were loaded in individual lanes of running gels, electrophoresed, and then transferred to membranes as described in the preceding section. Hsp70 was detected using 1:1000 dilutions of the mouse anti-human HSP 70 antibody (above) in overnight incubations at 4° C. After extensive washing, the secondary antibody (chicken anti-mouse IgG-HRP, Santa Cruz Biotechnology, Santa Cruz, Calif.) was used at 1:4000 dilutions, and subsequent steps and imaging were identical to that described previously. Relative densities of the Hsp70 bands were determined by calculating individual band intensities and areas, relative to proximate backgrounds, from digital images of the immunoblots, using a LAS-3000 Imaging System with Multi Gauge software (Fuji Film Digital Co., Stamford, Conn.). Respective band densities are expressed as standardized, arbitrary units.

Immunohistochemical Localization of Hsp70 in Lung Tissues: Snap frozen OCT embedded lung tissues were freshly cut and fixed with cold acetone and 5% formalin mixture for 3 minutes. Tissues were blocked with Peroxidase Blocking Kit (Dako, Glostrup, Denmark) and then normal horse serum for 30 minutes. Sections were incubated with mouse anti-human Hsp70 (Stressgen, Ann Arbor, Mich.) at 4° C. overnight. Following rinsing with PBS, the sections were incubated with biotinylated goat anti-mouse IgG (Vector Laboratories, Burlingame, Calif.) at room temperature for 1 hour, and then treated with AB Complex HRP (Vector Laboratories) for 30 min. Color was developed by adding ImmPACT DAB (Vector Laboratories) substrate for 1 minute. Images were captured with a photomicroscope (Zeiss, Thornwood, N.Y.) and scored (positive vs. negative) by two investigators blinded to subjects' clinical features and diagnoses.

Immunohistochemical Localization of IgG Immune Complexes in Lung Tissues: These methods have been described previously (16). In brief, frozen lung sections were cut and fixed in 2% PFA for 20 minutes. Sections were treated in 20 mM citrate buffer pH 6, at 95° C. for 10 minutes, blocked with normal serum for 30 minutes, and then incubated overnight with mouse (IgM) anti-human IgG (Serotec, Raleigh, N.C.) at 4° C. Tissues were rinsed and incubated with biotinylated goat anti-mouse IgM secondary antibody (Vector Laboratories) for one hour, and then rinsed with Tris buffer before adding AB Complex HRP (Dako) for 30 min. Color was developed by adding DAB substrate for 3-4 minutes. Microscopic images were taken in 6-9 randomly-selected fields from each specimen, and scored (positive vs. negative) by two investigators blinded to subjects' clinical features and diagnoses.

HLA Typing: HLA characterization of the initial IPF discovery cohort was performed by the Tissue Typing Laboratory at UPMC, using DNA isolated from leukocytes, in sequence specific oligonucleotide probe assays (Dynal RELI™ SSO, Invitrogen). HLA determinations for the UPMC normal control subjects were also made by sequence specific oligonucleotide probe assays, as previously described (32). HLA alleles among the NIH subjects were evaluated by polymerase chain reaction using sequence specific primers (PCR-SSP) (Invitrogen). The presence or absence of DRB1*15 among the Inova and Stanford transplantation recipients, and the ambulatory UPMC subjects (the autoantibody assay cohort) was determined by PCR-SSP (Qiagen, Valencia, Calif.), using DNA extracted from previously banked lung tissues or peripheral blood leukocyte pellets. Validation study of common specimens confirmed concordance of this methodology with the oligonucleotide probe assay results of the UPMC Tissue Typing laboratory.

Statistical Analysis: Unless denoted otherwise, two and three or more group comparisons of continuous variables were made by Mann-Whitney and Kruskal-Wallis tests, respectively. Dichotomous variables were analyzed by Chi-square. Survival analyses were performed using product-limit estimation, with comparisons by Log-rank (Mantel-Cox). Hazard ratios (HR) and 95% confidence intervals (CI) of censored data were established by proportional hazard regression model. Logistic regression analysis was used to generate odds ratios (OR) and 95% confidence intervals (CI) of uncensored dichotomous associations. Analyses were conducted with StatView v5.0.1 (SAS Institute, Cary, N.C.). Alpha (p) values<0.05 were considered significant. Data are depicted as means±SE.

### 6.2. Results

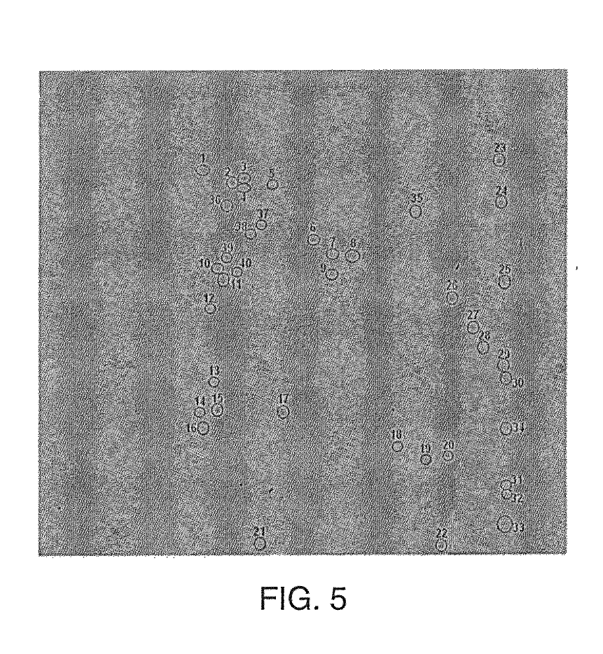

Autoantigen Identification Antigen discovery immunoprecipitation assays were performed three times. In each case, isoforms of Hsp70 were the most frequently identified putative autoantigen (FIG. 5). Hsp70 also seemed to have singular plausibility as a potential autoantigen, given multiple analogous descriptions in patients with various autoimmune disorders (19-22). Heat shock protein 27 (Hsp27) was also among other candidate autoantigens identified in these discovery assays (FIG. 5).

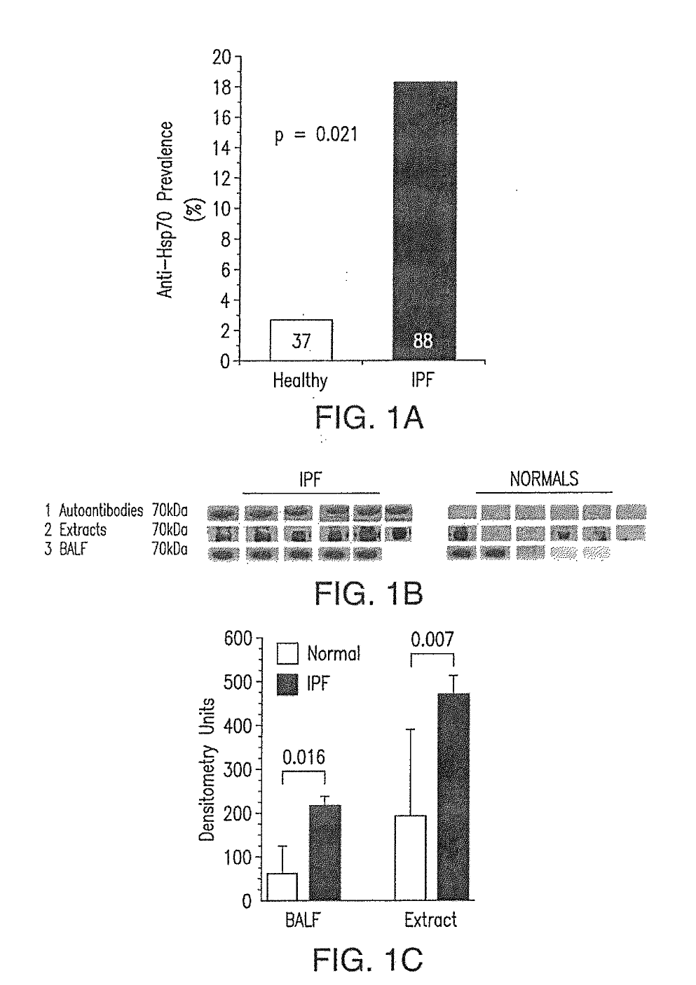

Circulating Anti-Hsp 70 Autoantibodies: Plasma from IPF patients and healthy normal controls were assayed for the presence of autoantibodies against Hsp70. Demographic characteristics of the IPF subjects are detailed in Table 1. The normal controls were comparable in terms of age (64 years old) and gender distribution (65% males). Plasma IgG autoantibodies were detected in 18.2% of the IPF patients vs. 2.7% of the healthy controls (FIG. 1A).

Hsp70 in Clinical Specimens: We hypothesized the biologic plausibility of Hsp70 as an autoantigen of IPF would be substantiated by finding this molecule is expressed in afflicted lungs. Hsp70 was present in all the IPF pulmonary specimens evaluated here by immunoblot (FIG. 1B). Hsp70 was less frequently detectible in the equivalent preparations from normal lung explants, and quantitation by densitometry corroborated these visualizations (FIG. 1C).

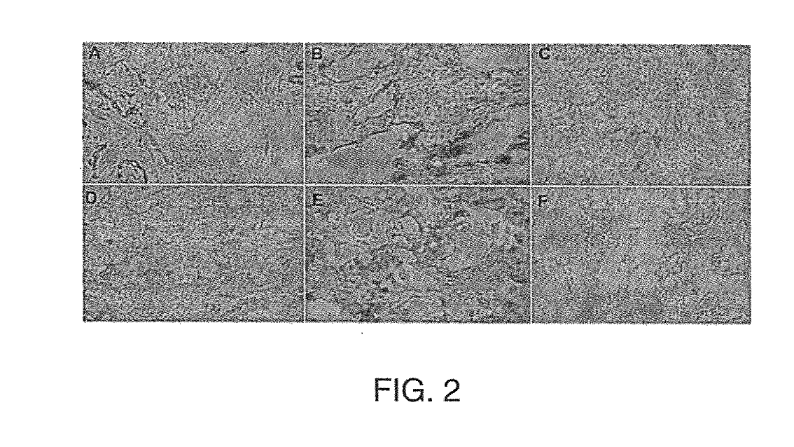

Immunohistochemistry was used to localize Hsp70 expression in situ in the IPF lung explants. Hsp70 was present in all six IPF lung lungs, and was especially prominent in distal airway epithelium, and to somewhat lesser extents in pneumocytes, alveolar macrophages and occasional endothelial cells (FIGS. 2A and 2B).

Intrapulmonary Immune Complexes: We have previously shown that IgG immune complex deposits are not typically present in normal lung explants (16). Those results were confirmed during the present investigations by the absence of these complexes in five of six additional normal lung explants examined here, and were only equivocally present in one other. In contrast, IgG complexes were overtly present in four of six (67%) of the IPF explants (FIGS. 2D and 2E) and equivocally present in another.

Clinical Correlates of Hsp70 Autoreactivity: We hypothesized that anti-Hsp70 IgG may play a role in IPF pathogenesis and, if so, the presence of these autoantibodies may associate with particular disease manifestations. Accordingly, physiologic and outcome measures were compared between the IPF patients with anti-Hsp70 autoantibodies (anti-Hsp70 Pos) and those who did not have these autoantibodies (anti-Hsp70 Neg).

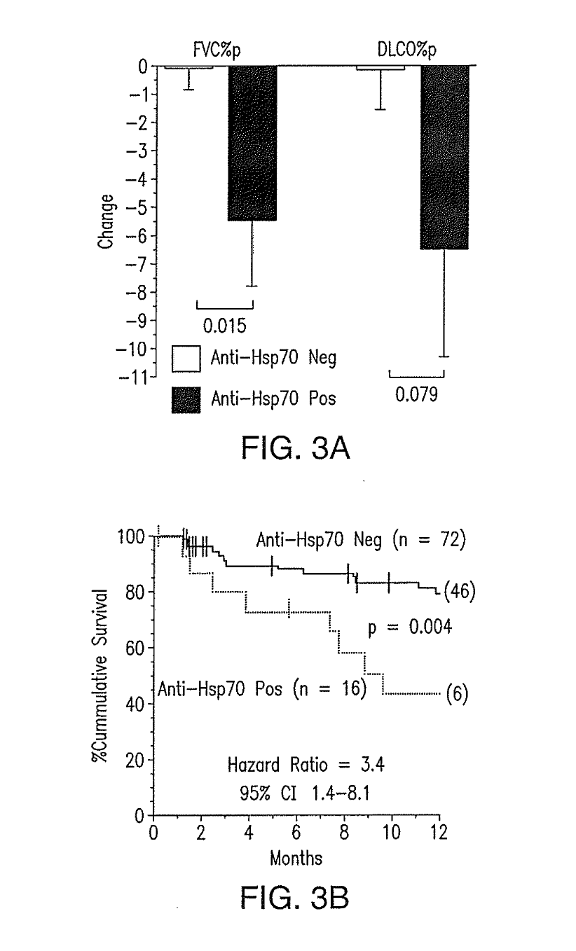

There were no significant differences of demographics or pulmonary function between the anti-Hsp70 Pos and anti-Hsp70 Neg subjects at the time of their specimen acquisitions (TABLE 1). Although the numbers of patients who had follow-up pulmonary function tests (PFT) after autoantibody specimen acquisitions were diminished due to attrition (deaths, lung transplantations, or becoming too ill to cooperate with the testing), there were nonetheless greater subsequent decrements of pulmonary function in the anti-Hsp70 Pos, compared to the anti-Hsp70 Neg patients (FIG. 3A).

The eventual progression of lung dysfunction in IPF patients almost inevitably results in either lung transplantation or the death of those afflicted (1). These dichotomous outcomes are not truly independent events, however, in that lung transplantation ostensibly diminishes or delays deaths of IPF patients, albeit by no means invariably, and the procedure itself is also associated with a finite inherent mortality and unique complications. Survival analyses, with lung transplantations tabulated as censored events (i.e., the end of observations), showed that one-year mortality was greater among the IPF patients with anti-Hsp70 autoantibodies (FIG. 3B). None of these patients were known to be having acute IPF exacerbations (13) at the time their plasma samples were obtained, and the intergroup survival difference appeared to be greatest several months after the anti-Hsp70 assays (FIG. 3B). Overt respiratory failure accounted for 78% of deaths among the anti-Hsp70 Pos and 55% of the anti-Hsp70 Neg mortality. Aside from one case of fatal pulmonary emboli among the latter, the other deaths in both subpopulations (all of whom had severe pulmonary disease) could not be assigned a specific proximate cause.

Equivalent minority proportions of IPF subjects in both anti-Hsp70 Pos and anti-Hsp70 Neg populations, most of who were recent referrals to UPMC, were taking immunosuppressants at the time of plasma specimen acquisitions (TABLE 1). Post hoc analyses limited to those patients not taking immunosuppressants showed anti-Hsp70 autoantibodies were present in 19% of these un-medicated subjects (p=0.018 compared to healthy, normal controls), and one-year mortality was 46% vs. 84% for the un-medicated anti-Hsp70 Pos and un-medicated anti-Hsp70 Neg patients, respectively (p=0.007).

Moreover, to exclude potential biasing by skewed selections of lung transplantation candidates, despite double blinding between the clinicians and laboratory investigators, we also performed post hoc survival analyses in which the transplantations were considered major adverse events equivalent to death. The one-year major adverse-event free survival of IPF patients who had anti-Hsp70 autoantibodies remained less than that of anti-Hsp70 Neg (38+12% vs. 64+6%, respectively, p=0.049).

Other Potential Humoral Autoantigens of IPF: Hsp27 was among other potential self-epitopes identified in one or more autoantigen discovery assays. Although annexin I was not identified in these immunoprecipitations, this protein was also of interest since it has been reported to be an autoantigen of IPF singularly associated with acute disease exacerbations (13).

Autoantibodies against Hsp27 and annexin I were present in only one of 12 (8.3%), and one of 15 (6.7%) IPF subjects tested, respectively, and further investigations in additional subjects did not seem to warrant depletion of these valuable clinical specimens. The two subjects who were positive for either annexin 1 or Hsp27 autoantibodies subsequently underwent lung transplantations within 2 and 9 months of their specimen acquisitions, respectively, and neither had autoantibodies against Hsp70.

HLA-DRB1*15 Prevalence: The initial compilation of HLA alleles for 79 UPMC lung transplantation recipients showed the allele prevalence of DRB1*15 (calculated as the proportion of subjects who have either one or two copies of this allele) was 37% among these IPF patients vs. 23% in a normal UPMC cohort of 196 subjects (p=0.02) (see TABLE 2). None of the other HLA Class II polymorphisms were significantly over-represented (TABLE 2).

The allele frequency of DRB1*15 (i.e., the total number of DRB1*15 alleles/total number of DRB1* alleles in the population) was 22% among the IPF subjects, which was also over-represented in comparisons to the DRB1*15 frequencies of 12% in the normal UPMC cohort (p=0.02), and 15% within a very large (n=6396) reference compilation of normal U.S. Caucasian subjects (35) (p=0.039).

Based on these initial findings, validation cohorts were prospectively analyzed (see details in TABLE 3), consisting of HLA data compiled from 35 IPF and 41 normal subjects at the NIH (34), and assays to detect DRB1*15 in tissue specimens from additional IPF patients at Inova (n 20) and Stanford (n=14). Of the 88 UPMC ambulatory IPF patients who had anti-Hsp70 assays, 13 had lung transplantations subsequent to their plasma acquisitions, and their HLA data had been previously tabulated in the UPMC discovery cohort. The remaining 75 IPF subjects with anti-Hsp70 autoantibody studies now had prospective DRB1*15 determinations using DNA extracted from their banked leukocyte specimens, and are included here as another validation cohort (see TABLE 3).

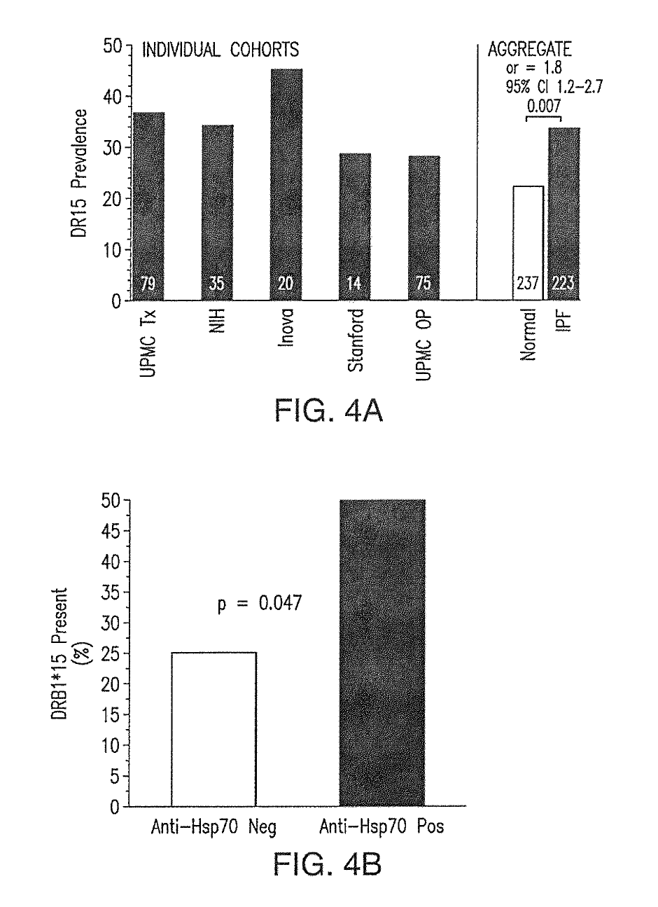

The aggregate prevalence of this allele within the cumulative IPF population was similar to that among the initial, discovery UPMC lung transplantation cohort, and was significantly greater than that of the normal controls (FIG. 4A).

Three IPF patients were known to have first-degree relatives who died with lung disease(s) that could possibly have been IPF. None of these patients had DRB1*15. Inasmuch as was known, the remainder of IPF cases were due to sporadic disease (1).

Approximately 95% of total DRB1*15 expression in Caucasians is attributable to the DRB1*1501 polymorphism, and this particular allele is also nearly in complete linkage disequilibrium (LD) with DQB1*0602 (35). DQB1*06 was also present in all of the DRB1*15+IPF subjects who also had typing at the DQB1* locus (i.e., the U. Pgh. and NIH cohorts). High-resolution DR determinations were also performed in the first 20 IPF transplantation recipients at the U. Pgh who were positive for DRB1*15, and in seven (7) of the NIH disease subjects with DRB1*15, and in every case these expressions were attributable to the DRB1*1501 polymorphism. Similarly, high resolution DQB1*06 characterizations were available for 11 of the U. Pgh. and five (5) of the NIH IPF patients who were known to express either DRB1*15 and/or DRB1*1501, and the DQB1*0602 polymorphism was present in all but one of these subjects.

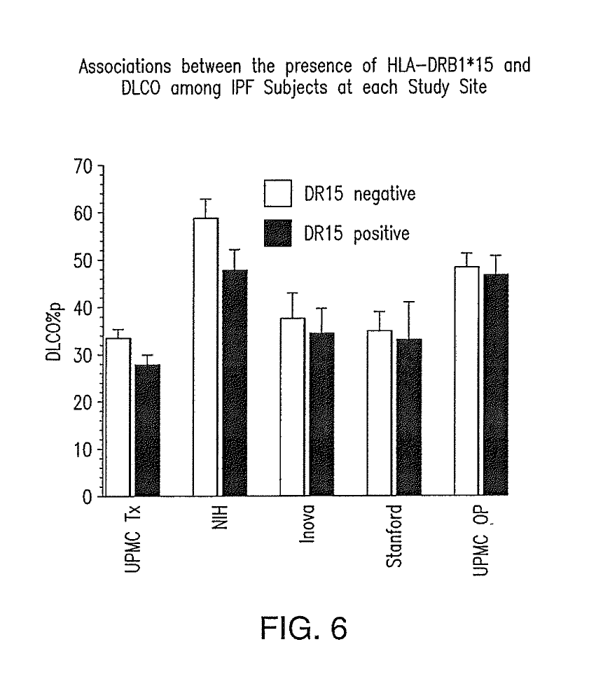

DRB1*15 Associations with Clinical Parameters: There were no evident associations between the presence (n=75) or absence (n=148) of DRB1*15 among the IPF patients and demographic characteristics or forced vital capacities (FVC), as a percentage of predicted values (FVC % p) (see TABLE 4). Predicted values of single-breath diffusing capacities for carbon monoxide (DLCO % p) were comparatively decreased among the DRB1*15 Positive subjects (TABLE 4), a consistent trend among the subjects at each study site, and despite the often considerable differences in the severity of lung disease among these respective study cohorts (see FIG. 6). This DLCO % p difference seemed unlikely to be a cryptic result of smoking per se, or a secondary effect of underlying pulmonary artery (PA) hemodynamics (36), given similar smoking histories and PA pressures among both DRB1*15 Positive and DRB1*15 Negative subpopulations (TABLE 4).

Perhaps more importantly, however, there was an association between the common presences of DRB1*15 and autoantibodies against Hsp70 among the 88 IPF subjects in whom both DNA and plasma specimens were accessible for study (the ambulatory UPMC cohort) (FIG. 4B). Those IPF patients who had anti-Hsp70 autoantibodies but lacked the DRB1*15 allele were all DQB1*03 positive, although the association between the latter, frequently expressed HLA polymorphism, and the presence of anti-Hsp70 responses did not reach significance.

### 6.3. Discussion

These data show that circulating anti-Hsp 70 IgG autoantibodies are present in a subpopulation of IPF patients who are destined for greater progression of pulmonary dysfunction and increased mortality (FIGS. 3A and 3B). Other data here also show that Hsp70, the self-epitope of this autoimmune response, is expressed in the lungs of IPF patients (FIGS. 1B, 1C, 2A, and 2B). Additional assays demonstrate IgG-antigen complex deposits, one of the pathogenic consequences of autoantibody responses (17), are common in end-stage IPF lungs (FIGS. 2D and 2E). Furthermore, the HLA Class II allele DRB1*15 was shown here to be over-represented among Caucasian IPF subjects with varying disease severities from four U.S. medical centers (FIG. 4A). This particular HLA polymorphism is also associated with the presence of anti-Hsp70 autoantibody responses in IPF patients (FIG. 4B), and similar HLA allele-autoantibody correlations are a common finding of autoimmune diseases (23, 32, 33, 37). These data are also consistent with a general hypothesis that the development of IPF is likely to involve interactions between cryptic environmental agent(s) and genetic factors (in this case, immunogenetic elements of the HLA Class II region) (38). This environment-gene interaction disease paradigm is common to many other disorders, notably including those characterized by autoimmunity (33,37).

Studies of clinical specimens have shown that numerous adaptive immune abnormalities are present in IPF patients. The majority of IPF patients have antibodies against a variety of self-antigens that are typically distinct from those that define classical autoimmune syndromes (e.g., scleroderma, systemic lupus erythematosus [SLE]) (6-14). Furthermore, the presence of IgG autoantibodies in IPF patients is also indirect, but nonetheless compelling, evidence of T-cell involvement in this disease, since the production of IgG with avidity for protein epitopes is dependent on the provision of facultative help by CD4 T-cells that have specificity for those particular antigens (31). Other studies have directly shown that T-cells among IPF patients are abnormally activated, have enhanced production of diverse inflammatory and/or pro-fibrotic mediators, and impaired regulatory (Treg) function (14, 28, 29). First-degree relatives of patients with familial IPF have intrapulmonary infiltrations of activated CD4 T-cells many years prior to the development of clinically-evident lung abnormalities (27). Findings of extensive oligoclonal CD4 T-cell proliferations in the lungs and periphery of those afflicted are specific evidence that a restricted number of conventional peptide antigens drive repetitive lymphocyte activations in IPF patients (14,39). Furthermore, abnormal proportions of phenotypically and functionally distinct CD4+CD28null T-cells, the daughter progeny of repetitive antigen-driven lymphocyte proliferations, and a highly specific feature of chronic immunologic disorders, are also present in the circulation (and lungs) of IPF patients, and the extent of this CD4 T-cell alteration is highly associated with clinical outcomes (29).

Autoreactivity to various Hsp (15,19-22) is not necessarily due to a seminal, de novo loss of self-tolerance for these ubiquitous proteins. Hsp expression is up-regulated by a wide variety of tissue injuries and cellular stresses, including immunological responses originally directed at other antigens. High concentrations of Hsp in proximity to active, ongoing immunologic processes appear to be conducive to promotion of reactivity against these carrier proteins per se, even if the immune response was originally targeted at other determinants, including antigens among the transported peptides (19-22). In addition to other potential paradigms, microbial Hsp have been implicated as inciting factors for development of Hsp autoreactivity in their human hosts (40), presumably a result of epitope spread from the initial anti-microbial immune response (37,41), or cross-reactivity (“mimicry”) between microbial and self-Hsp antigenic determinants (42). The latter mechanism may be especially plausible because many Hsp are highly conserved among diverse species. By whatever processes, Hsp70 in particular has been implicated as a self-antigen in a variety of autoimmune disorders, including Type 1 diabetes, autoimmune hepatitis, multiple sclerosis, and juvenile arthritis (19-22).

The most straightforward explanation of the frequent association between unique HLA polymorphisms and immunologic syndromes (23-26, 32, 33, 37) may also relate to nuances of antigen presentation. HLA molecules are requisite effectors for presentations of peptide antigens to the T-cells that initiate adaptive immune responses, but each distinct HLA allele has a restricted peptide [antigen] binding motif (30). Hence, HLA haplotype inheritance determines the finite repertoire of antigens that can evoke T-cell responses in an individual. Although critical for host defense, these adaptive immune responses may be deleterious if, as an example, the antigen is a self-protein (autoantigen), or one that evokes a cross-response to a self-protein by mimicry or epitope spread (33, 37, 40-43). In contrast, individuals lacking these specific, “permissive” HLA alleles do not present those particular disease-associated epitopes, and do not initiate the deleterious response(s). Of perhaps some relevance with respect to the present observations, HLA-DRB1*15 is also one of the most frequently over-represented HLA Class II alleles among those afflicted by various immunologic diseases, including multiple sclerosis, sarcoidosis, SLE, and Goodpasture's syndrome (18, 23-26). The latter two entities may also be of special pertinence to the current findings because the pathogenesis of those syndromes is also generally believed to be autoantibody-mediated (18, 33, 43).

While our findings here of HLA Class II DRB1*15 over-representation in IPF patients and concurrent association of this allele with a specific autoimmune response are consistent with a direct role (e.g., unique antigen presentation) of this allele in CD4 T-cell pathogenesis (31), these data cannot exclude the possibility that the element(s) responsible for this disease association could be another immunoregulatory gene in LD with DRB1*15. The human major histocompatibility (MHC) complex on chromosome 6p21.31 is typified by the presence of numerous, extraordinarily polymorphic HLA alleles, as well as other immunoregulatory genes that are in very strong LD (23, 24, 26), rendering evaluation of this region extremely problematic by associative methodologies (e.g., genome wide association studies [GWAS]). Based on previous HLA allele and haplotype characterizations within large series and reference populations (23, 24, 26, 35), as well as the limited higher resolution determinations available here, it is overwhelmingly likely that the actual HLA DRB1*15 polymorphism over-represented in IPF subjects is DRB1*1501. This particular allele is also in nearly complete LD with DQA1*0102 and DQB1*0602 among Caucasians, and is the single most frequent HLA Class II haplotype in this racial group (35). Because of the very strong LD within this haplotype, it is difficult to precisely identify the particular disease-associated HLA allele among them, or distinguish the contributions of these HLA from other proximate immunoregulatory elements, in lieu of focused, high-resolution genomic studies (e.g., haplotype sequencing) (21).

Almost all previous HLA characterizations of IPF patients date from the relatively distant past, before the development of precise molecular methodologies to unequivocally define and distinguish these alleles (44-49). Moreover, only two of those early serologic-based determinations (48,49) examined even a very restricted repertoire of the many, since-discovered, HLA Class II alleles (TABLE 5). Diagnostic criteria for IPF have also evolved considerably during the intervening years (1), raising potential concerns about the case definitions of the earlier study populations. The numbers of subjects among those investigations were also usually quite small, severely limiting their power to detect intergroup differences (see TABLE 5). Despite these potential limitations, however, several earlier reports indicated HLA allele frequency perturbations may be present in IPF (44, 47-49), although this finding was not invariable (45,46).

To our knowledge, contemporary analogous analyses using molecular techniques and current IPF case definitions are limited to a single cohort study of Mexican patients that reported various HLA Class II alleles, including DRB1*01, DRB1*04, and DRB1*14, were over-represented in that IPF population (50). We did not see abnormal frequencies of those particular DRB1 alleles in our IPF patients (see TABLE 2). Conversely, the frequencies of DRB1*15 alleles in both the IPF and normal control populations of that previous study (50) were several-fold less than that measured presently, and were also much less than the reported frequencies within other large Caucasian control populations (24, 26, 35). The seeming discrepancy between that previous report (50) and the present findings may possibly be attributable to the often considerable variability of HLA allele frequencies among different races and ethnicities (35).

The present data cannot also absolutely prove that anti-Hsp70 immunologic responses contribute to the lung injury of IPF. Conclusive demonstrations of human autoimmune pathogenesis are extremely difficult (and often impossible) to devise, if for no other reason than complete fulfillment of Koch's postulates (e.g., infusions of autoantibodies into normal individuals to recreate the disease) would be logistically difficult and morally unacceptable. For this and other reasons, the mechanistic determinants of tissue damage in many well-known conventional autoimmune disorders still remain largely unknown, despite intense study over many years (37,43). Nonetheless, autoimmune pathogenicity is highly probable, and generally recognized as such, for syndromes wherein potentially pathogenic autoantibodies (or autologous T-cells) have avidity for antigens within the diseased tissue(s) (FIGS. 1A-C, 2A and 2B), immune-mediated pathologic processes are evident within the afflicted organ (e.g., immune complex deposits) (FIGS. 2D and 2E), and the presence of these specific immune effectors is associated with clinical manifestations of the disease (FIGS. 3A and 3B). The unique associations here of anti-Hsp70 autoreactivity with both clinical manifestations and a distinct, over-represented HLA Class II allele (FIG. 4) also support the pathogenic relevance of the present observations. Thus, Hsp70 autoreactivity in IPF patients does not appear to merely be a coincidental reflection of a nonspecific (if heretofore unrecognized) inflammatory condition characterized by polyclonal production of promiscuously-avid immunoglobulins. As such, the findings here and elsewhere (11-14,29) fulfill criteria that establish the presence of a clinically-relevant immunologic process(es) in disease progression (33, 37, 43).

TABLE 1
Demographic and Clinical Characteristics of IPF Subjects with
Autoantibody Studies

| | Anti-Hsp70 Neg | Anti-Hsp70 Pos | Aggregate |
|---|---|---|---|
| n | 72 | 16 | 88 |
| Age (yrs) | 70 ± 1 | 69 ± 2 | 70 ± 1 |
| Gender (% male) | 70 | 71 | 70 |
| Lung Biopsy (%) | 60 | 56 | 59 |
| FVC % predicted | 62.8 ± 2.4 | 57.3 ± 4.5 | 61.8 ± 2.1 |
| DL_{CO} % predicted | 47.0 ± 2.5 | 45.9 ± 5.1 | 46.8 ± 2.2 |
| Immunosuppressants (%) | 22 | 19 | 22 |

Anti-Hsp70 Neg and Anti-Hsp70 Pos denote IPF subjects who do not have or do have circulating anti-Hsp70 autoantibodies, respectively. Lung biopsies (or histologic evaluations of explants) in all cases showed either usual interstitial pneumonia or end-stage fibrotic disease. Immunosuppressants denote subjects taking any single agent or various permutations of prednisone (5-20 mg/day), azathioprine, interferon-gamma, mycophenolate, or tacrolimus. None of the intergroup differences were significant.

TABLE 2
HLA Class II Allele Frequencies in the Initial IPF Cohort

| | IPF Frequency | Control Frequency |
|---|---|---|
| DQB1* Alleles | | |
| 02 | 29.1 | 40.6 |
| 03 | 54.4 | 55.7 |
| 04 | 3.8 | 7.8 |
| 05 | 29.1 | 31.8 |
| 06 | 51.9 | 40.1 |
| DRB1* Alleles | | |
| 01 | 17.7 | 21.4 |
| 03 | 20.3 | 16.3 |
| 04 | 29.1 | 28.1 |
| 07 | 21.5 | 26.5 |
| 08 | 3.8 | 8.7 |
| 09 | 1.3 | 1.0 |
| 10 | 2.5 | 1.0 |
| 11 | 16.5 | 20.9 |
| 12 | 2.5 | 3.1 |
| 13 | 24.1 | 26.0 |
| 14 | 5.1 | 3.6 |
| 15 | 36.7* | 23.0 |
| 16 | 3.8 | 5.1 |

HLA allele prevalences (the percentages of subjects with one or more copies of the allele) in the initial UPMC lung transplant recipient population of IPF patients (n=79) were compared to those of a normal reference population (n=196) previously characterized in the context of other studies at this medical center (32). DRB1*15 was the most over-represented of the common HLA Class II alleles among the IPF, relative to the controls. These initial findings prompted further study by recruitments of IPF validation cohorts from three other medical centers (see text). **p=0.02. None of the other HLA allele comparisons here were significant.

TABLE 3
Characteristics of the IPF Subjects who had HLA Studies

| | UPMC | | | | UPMC | |
|---|---|---|---|---|---|---|
| | Tx | NIH | Inova | Stanford | OP | Aggregate |
| n | 79 | 35 | 20 | 14 | 75 | 223 |
| Age (years)* | 67 ± 1 | 63 ± 1 | 58 ± 1 | 59 ± 2 | 70 ± 1 | 66 ± 1 |
| Males (%) | 75 | 69 | 65 | 57 | 71 | 70 |
| FVC % p** | 49.8 ± 1.7 | 74.0 ± 3.5 | 54.5 ± 3. [symbol] | 54.9 ± 4.9 | 63.0 ± 2.3 | 58.7 ± 1.3 |
| DL_{CO} % p** | 31.4 ± 1.4 | 53.6 ± 3.1 | 36.1 ± 3. [symbol] | 34.5 ± 3.6 | 47.8 ± 2.4 | 41.2 ± 1.3 |
| Lung | Yes | No | Yes | Yes | No | No/Yes |
| transplant | | | | | | |
| recipients | | | | | | |

[symbol]

indicates data missing or illegible when filed

UPMC Tx denotes the initial discovery cohort for HLA allele frequency determinations, consisting of subjects who had lung transplantations for IPF at the University of Pittsburgh Medical Center (see also TABLE 2). The subsequent validation (replication) cohorts include ambulatory subjects from the National Institute of Health (NIH), and transplant recipients from Inova Fairfax, and Stanford. UPMC OP denotes Pittsburgh IPF subjects who initially had plasma specimens acquired in the context of autoantibody studies. These subjects were ambulatory (Out-Patients) at the time of their initial recruitment for those immunologic studies. Thirteen of the original 88 subjects with these autoantibody assays subsequently had lung transplantations, and their HLA data had already been tabulated in the UPMC Tx cohort. FVC % p denotes forced vital capacity, as a percentage of predicted normal values; DL_{CO}% p denotes single-breath diffusing capacity for carbon monoxide as a percentage of predicted normal values. Pulmonary function test (PFT) values among UPMC Tx, Inova, and Stanford subjects are based on last determinations prior to their lung transplantations. PFT values of the ambulatory patients here (NIH and UPMC OP) were compiled at the time of their plasma specimen acquisitions. *p<0.0001, by factorial ANOVA, with significant intergroup differences by post-hoc multiple comparisons (Bonferroni-Dunn) of both UPMC Tx and UPMC OP with the other subpopulations. **p<0.0001, by factorial ANOVA, with significant intergroup differences by post-hoc multiple comparisons (Bonferroni-Dunn) between the NIH vs. all other supopulations, as well as between UPMC Tx and UPMC OP.

TABLE 4
Associations between the presence of HLA-DRB1*15 and
Clinical/Demographic Characteristics of IPF Subjects

| | HLA-DRB1*15 Negative | HLA-DRB1*15 Positive |
|---|---|---|
| n | 148 | 75 |
| Age (yrs) | 66 | 65 |
| % males | 70 | 71 |
| FVC % predicted | 59.5 ± 1.7 | 57.1 ± 2.1 |
| DL_{CO} % predicted* | 43.0 ± 1.6 | 37.6 ± 2.0 |
| Smoking History (%) | 61 | 64 |
| PA systolic (mm Hg) | 41.5 ± 1.4 | 40.9 ± 2.1 |
| PA diastolic (mm Hg) | 15.0 ± 0.8 | 13.6 ± 1.1 |
| PA mean (mm Hg) | 25.7 ± 1.0 | 25.3 ± 1.4 |
| PA Hypertension (%) | 47 | 47 |

HLA-DRB1*15 Negative and Positive denote, respectively, IPF subjects without or with the presence of at least one HLA-DRB1*15 allele. Pulmonary artery (PA) pressures were obtained in all but seven (7) of the IPF subjects who were undergoing evaluations for lung transplantations (n=65 and 40, for HLA-DRB1*15 Negative and HLA-DRB1*15 Positive, respectively), prior to these surgical procedures. PA Hypertension is defined here as PA means>25 mm Hg with PA capillary pressures <15 mmHg. *p=0.046, by unpaired t-test.

TABLE 5
Previous studies of HLA allele frequencies in IPF

| | | | | Abnormal HLA | | |
|---|---|---|---|---|---|---|
| | #Alleles | Molec- | | allele frequency | | |
| Loci | tested | ular | n | in IPF? | year | ref |
| ? | ? | No | 20* [symbol] | Yes HLA12 | 1976 | 44 |
| -A, -B | 24 | No | 32 | no | 1977 | 45 |
| -A, -B | 35 | No | 33 | no | 1978 | 46 |
| -A, -B, | 36 | No | 50 | Yes: B8 | 1978 | 47 |
| -C | | | | | | |
| -A, -B, | 32 | No | 38* [symbol] | Yes: B15 and | 1979 | 48 |
| -C -Dw | 4 | | | Dw6 | | |
| -A, -B, | 65 (total in | No | 20 | Yes: DR2 | 1983 | 49 |
| -C, -DR | all loci) | | | | | |
| -A, -B, | 45 | Yes | 75 | Yes, multiple | 2008 | 50 |
| -DR, -DQ | multiple | | | Class I and | | |
| | | | | Class II | | |
| | | | | alleles and | | |
| | | | | haplotypes (but | | |
| | | | | not DRB1*15) | | |

[symbol]

indicates data missing or illegible when filed

This study also included patients with other, known, conventional autoimmune syndromes. Approximately 50 HLA-A, 85 HLA-B, 45 HLA-C, 44 HLA-DR, and 16 HLA-DQ distinct polymorphisms (alleles and suballeles) are known to be expressed in Caucasian populations (35).

### 6.4 REFERENCES

1. American Thoracic Society. Idiopathic pulmonary fibrosis: diagnosis and treatment. International consensus statement. American Thoracic Society (ATS), and the European Respiratory Society (ERS). Am J Respir Crit Care Med. 161, 646-664 (2000)
2. D. A. Campbell, L. W. Poulter, G. Janossy, R. M. du Bois. Immunohistological analysis of lung tissue from patients with cryptogenic fibrosing alveolitis suggesting local expression of immune hypersensitivity. Thorax. 40, 405-11 (1985)
3. J. Marchal-Somme, Y. Uzunhan, S. Marchand-Adam, D. Valeyre, V. Soumelis, B. Crestani, P. Soler. Cutting edge: non-proliferating mature immune cells form a novel type of organizing lymphoid structure in idiopathic pulmonary fibrosis. J Immunol. 176, 5735-5739 (2006)
4. F. Zuo, N. Kaminski, E. Eugui, J. Allard, Z. Hakhini, A. Ben-Dor, L. Lollini, D. Morris, Y. Kim, B. DeLustro, D. Sheppard, A. Pardo, M. Selman, R. A. Heller. Gene expression analysis reveals matrilysin as a key regulator of pulmonary fibrosis in mice and humans. Proc Natl Acad Sci., USA, 99, 6292-97 (2002)
5. P. P. Dall Aglio, A. Pesci, G. Bertorelli, E. Brianti, S. Scarpa. Study of immune complexes in broncholaveolar lavage fluids. Respiration. 54, 36-41 (1988)
6. N. Dobashi, J. Fujita, M. Murota, Y. Ohtsuki, I. Yamadori, T. Yoshinouchi, R. Ueda, S. Bandoh, T. Kamei, M. Nishioka, T. Ishida, J. Takahara. Elevation of anti-cytokeratin 18 antibody and circulating cytokeratin 18: anti-cytokeratin 18 antibody immune complexes in sera of patients with idiopathic pulmonary fibrosis. Lung. 178, 171-9 (2000)
7. B. Grigolo, I. Mazzetti, R. M. Borzi, H. D. Hickson, M. Fabbri, L. Fasano, R. Meliconi, A. Facchini. Mapping of topoisomerase II alpha epitopes recognized by autoantibodies in idiopathic pulmonary fibrosis. Clin Exp Immunol. 114, 339-46 (1998)
8. Y. Yang, J. Fujita, S. Bandho, Y. Ohtsuki, I. Yamadori, T. Yoshinouchi, T. Ishida. Detection of antivimentin antibody in sera of patients with idiopathic pulmonary fibrosis and non-specific interstitial pneumonia. Clin Exp Immunol. 128, 169-74 (2002)
9. T. Takahashi, I. Wada, Y. Ohtsuka, M. Munakata, Y. Homm, Y. Kuroki. Autoantibody to alanyl-tRNA synthetase in patients with idiopathic pulmonary fibrosis. Respirology. 12, 642-653 (2007)
10. W. H. H. Wallace, S. M. Howie. Upregulation of tenascin and TGF-□ production in a type H alveolar epithelial cell line by antibody against a pulmonary auto-antigen. J Pathol. 195, 251-6 (2001)
11. C. M. Magro, W. J. Waldman, D. A. Knight, J. N. Allen, T. Nadasdy, G. E. Frambach, P. Ross, C. B. Marsh. Idiopathic pulmonary fibrosis related to endothelial injury and antiendothelial cell antibodies. Hum Immunol. 67, 284-297 (2006)
12. F. Ogushi, K. Tani, T. Endo, H. Tada, T. Kawano, T. Asano, L. Huang, Y. Ohmoto, M. Muraguchi, H. Moriguchi, S. Sone. Autoantibodies to IL-1□ in sera from rapidly progressive idiopathic pulmonary fibrosis. J Med Invest. 48, 181-9 (2001)
13. K. Kurosu, Y. Takiguchi, O. Okada, N. Yumoto, S. Sakao, Y. Tada, Y. Kasahara, N. Tanabe, K. Tatsumi, M. Weiden, W. N. Rom, T. Kuriyama. Identification of annexin 1 as a novel autoantigen in acute exacerbation of idiopathic pulmonary fibrosis. J Immunol. 181, 756-767 (2008)
14. C. A. Feghali-Bostwick, C. G. Tsai, V. G. Valentine, S. Kantrow, M. W. Stoner, J. M. Pilewski, A. S. Gadgil, M. P. George, K. F. Gibson, A. M. Choi, N. Kaminski, Y. Zhang, S. R. Duncan. Cellular and humoral autoreactivity in idiopathic pulmonary fibrosis. J Immunol. 179, 2592-9 (2007)
15. M. Gonzalez-Grwonow, M. Cuchacovich, C. Llanos, C. Urzua, G. Gawdi, S. V. Pizzo. Prostate cancer cell proliferation in vitro is modulated by antibodies against glucose-regulated protein 78 isolated from patient serum. Cancer Res. 66, 11424-11431 (2006)
16. C. A. Feghali-Bostwick, A. S. Gadgil, L. E. Otterbein, J. M. Pilewski, M. W. Stoner, E. Csizmadia, Y. Zhang, F. C. Sciurba, S. R. Duncan. Autoantibodies in patients with chronic obstructive pulmonary disease. Am J Resp Critical Care Med. 177, 156-163 (2008)
17. T. N. Mayada, G. C. Tsokos, N. Tsuboi. Mechanisms of immune complex-mediated neutrophil recruitment and tissue injury. Circulation. 120, 2012-2024 (2009)
18. S. B. Erickson, S. B. Kurtz, J. V. Donadio, K. E. Holley, C. B. Wilson, A. A. Pineda. Use of combined plasmapharesis and immunosuppression in the treatment of Goodpasture's syndrome. Mayo Clin Proc. 54, 714-720 (1979)
19. Z. Prohászka. Chaperones as part of immune networks. Adv Exp Med Biol. 594, 159-66 (2007)
20. F. Tahiri, F. Le Naour, S. Huguet, R. Lai-Kuen, D. Samuel, C. Johanet, B. Saubamea, V. Tricottet, J. C. Duclos-Vallee, E. Ballot. Identification of plasma membrane autoantigens in autoimmune hepatitis type 1 using a proteomics tool Hepatology. 47(3), 937-48 (2008)
21. D. Zlacka, P. Vavrincova, T. T. Hien Nguyen, I. Hromadnikova. Frequency of anti-hsp60, -65 and -70 antibodies in sera of patients with juvenile idiopathic arthritis. J Autoimmun. 27(2), 81-8 (2006 Sep. 27) Epub (2006 Aug. 24).
22. R. Abulafia-Lapid, D. Gillis, O. Yosef, H. Atlan, I. R. Cohen IR. T cells and autoantibodies to human HSP70 in Type 1 diabetes in children. J Autoimmun. 20, 313-321 (2003)
23. P. I. de Bakker, G. McVean, P. C. Sabeti, M. M. Miretti, T. Green, J. Marchini, X. Ke, A. J. Monsuur, P. Whittaker, M. Delgado, J. Morrison, A. Richardson, E. C. Walsh, X. Gao, L. Galver, J. Hart, D. A. Hafler, M. Pericak-Vance, J. A. Todd, M. J. Daly, J. Trowsdale, C. Wijmenga, T. J. Vyse, S. Beck, S. S. Murray, M. Carrington, S. Gregory, P. Deloukas, J. D. Rioux. A high-resolution HLA and SNP haplotype map for disease association studies in the extended human MHC. Nature Genetics. 38, 1166-1172 (2006)
24. H. Schmidt, D. Williamson, A. Ashley-Koch. HLA-DR15 haplotype and multiple sclerosis: a huge review. Am J Epidemiol. 165, 1097-1109 (2007)
25. F. Takeuchi, K. Nakano, H. Nabeta, G. H. Hong, K. Kawasugi, M. Mori, H. Okudaira, S. Kuwata, K. Tanimoto. Genetic contribution of the tumor necrosis factor (TNF) B+252*2/2 genotype, but not the TNFa,b microsatellite alleles, to systemic lupus erythematosis in Japanese patients. Int J Immunognet. 32, 173-178 (2005)
26. C. E. M. Voorter, M. Drent, E. M. van den Berg-Loonen. Severe pulmonary sarcoidosis is strongly associated with the haplotype HLA-DQB1*0602-DRB1*1501. Hum Immunol. 66, 826-835 (2005)
27. I. O. Rosas, P. Ren, N. A. Avila, C. K. Chow, T. J. Franks, W. D. Travis, J. P. McCoy Jr, R. M. May, H. P. Wu, D. M. Nguyen, M. Arcos-Burgos, S. D. MacDonald, B. R. Gochuico. Early interstitial lung disease in familial pulmonary fibrosis. Am J Resp Crit Care Med 176, 698-705 (2007)
28. I. Kotsianidis, E. Nakou, I. Bouchliou, A. Tzouvelekis, E. Spanoudakis E, P. Steiropoulos, I. Sotiriou, V. Aidinis, D. Margaritis, C. Tsatalas, D. Bouros. Global impairment of CD4+CD25+FoxP3+ regulatory T cells in idiopathic pulmonary fibrosis. Am J Resp Crit Care Med. 179, 1121-1130 (2009)
29. S. R. Gilani, L. J. Vuga, K. O. Lindell, K. F. Gibson, J. Xue, E. K. Lindsay, N. Kaminski, V. G. Valentine, M. P. George, C. Steele, S. R. Duncan. CD28 down-regulation on circulating CD4 T-cells is associated with poor prognoses of patients with idiopathic pulmonary fibrosis. Plos One. 5:e8959 (2010)
30. M. G. Rudolph, R. L. Stanfield, I. A. Wilson. How TCRs bind MHCs, peptides, and coreceptors. Annu Rev Immunol. 24, 419-466 (2006)
31. D. C. Parker. T-cell dependent B-cell activation. Annu Rev Immunol. 11, 331-340 (2006)
32. D. Falkner, J. Wilson, N. Fertig, K. Clawson, T. A. Medsger, P. A. Morel. Studies of HLA-DR and DQ alleles in SSc patients with autoantibodies to RNA polymerases and U3-RNP (fibrillarin). J Rheum. 27, 1196-1201 (2000)
33. P. Marrack, J. Kappler, B. L. Kotzin. Autoimmune disease: why and where it occurs. Nat Immunol. 7, 899-905 (2001)
34. P. Ren, I. O. Rosas, S. D. MacDonald, H. P. Wu, E. M. Billings, B. R. Gochuico. Impairment of alveolar macrophage transcription in idiopathic pulmonary fibrosis. Am J Respir Crit Care Med. 175, 1151-7 (2007)
35. National Bone Marrow Donor Program. Haplotype Frequencies. Accessed Nov. 10, 2009 at https://bioinformatics.nmdp.org.
36. C. J. Lettieri, S. D. Nathan, S. D. Barnett, S. Ahmad, A. F. Shorr. Prevalence and outcomes of pulmonary arterial hypertension in advanced idiopathic pulmonary fibrosis. Chest. 129, 746-752 (2006)
37. J. Ermann, C. G. Fathman. Autoimmune diseases: genes, bugs, and failed regulation. Nat Immunol. 2, 759-761 (2001)
38. J. C. Grutters, R. M. du Bois. Genetics of fibrosing lung diseases. Eur Resp J. 25, 915-927 (2005)
39. A. Shimizudani, H. Murata, H. Keino, S. Kojo, H. Nakamura, Y. Morishima, T. Sakamoto, M. Ohtsuka, K. Sekisawa, M. Sumida, T. Sumida, T. Matsuoka. Conserved CDR 3 region of T cell receptor BV gene in lymphocytes from bronchoalveolar lavage fluid of patients with idiopathic pulmonary fibrosis. Clin Exp Immunol. 129, 140-149. (2002)
40. S. Hoshida, M. Nishion, J. Tanouchi, T. Kishimoto, Y. Yamad. Acute Chlamydia pneumonia infection with heat shock protein 60-related response in patients with acute coronary syndromes. Atherosclerosis. 183, 109-112 (2005)
41. C. L. Vanderlugt, S. D. Miller. Epitope spreading in immune mediated diseases: implications for immunotherapy. Nat Rev Immunol. 2, 85-94 (2002)
42. M. B. Oldstone. Molecular mimicry, microbial infection and autoimmune disease: evolution of the concept. Curr Top Microbiol Immunol. 296, 1-17 (2005)
43. P. E. Lipsky. Systemic lupus erythematosus: an autoimmune disease of B cell hyperactivity. Nat Immunol. 2, 764-766 (2001)
44. C. Evans. HLA antigens in diffuse fibrosing alveolitis. Thorax 1976; 31:483-5
45. C. V. Strimlan, H. F. Taswell, R. A. DeRemee, F. Kueppers. HLA antigens and fibrosing alveolitis. Am Rev Resp Dis. 1120-1 (1977)
46. J. D. Fulmer, M. S. Sposovska, E. R. von Gal, R. G. Crystal, K. K. Mittal. Distribution of HLA antigens in idiopathic pulmonary fibrosis. Am Rev Resp Dis. 118, 141-47 (1978)
47. C. W. G. Turton, L. M. Morris, S. D. Lawler, M. Turner-Warwick. HLA in cryptogenic fibrosing alveolitis. Lancet. 1(8062), 507-8 (1978)
48. E. Varpela, A. Tiilkainen, M. Varpela, P. Tukiainen. High prevalences of HLA-B15 and HLA-Dw6 in patients with cryptogenic fibrosing alveolitis. Tissue Antigens. 14, 68-71 (1979)
49. D. M. Libby, A. Gibofsky, M. Fotino, S. J. Waters, J. P. Smith. Immunogenetic and clinical findings in idiopathic pulmonary fibrosis. Am Rev Resp Dis. 127, 618-22 (1983)
50. R. Falfan-Valenci, A. Camarena, A. Juarez, C. Becerril, M. Montano, J. Cisneros, F. Mendoza, J. Granados, A. Pardo, M. Selman. Major histocompatibility complex and alveolar epithelial apoptosis in idiopathic pulmonary fibrosis. Hum Genet. 118, 235-244 (2005).

### 7. Example

### Idiopathic Pulmonary Fibrosis Patients Who have Autoantibodies to Heat Shock Protein 70 have More Rapid Clinical Deterioration and Greater Mortality

### 7.1. Materials and Methods

Specimens for Autoantibody Studies: Plasma specimens for autoantibody studies were obtained by centrifugation of anti-coagulated peripheral blood from consecutive ambulatory IPF patients. At least six (6) months of subsequent observation from the time of specimen acquisitions was available on all IPF subjects who did not die or undergo lung transplantation during that interval. COPD plasma specimens were collected from ambulatory subjects in an ongoing longitudinal study (1P50 HL084948-01). Control plasma for autoantibody assays was procured from among the normal volunteers of that COPD study or recruited from hospital personnel by solicitation. All plasma specimens were stored at −80° C. until batch processing and use in these assays. Peripheral blood mononuclear cells (PBMNC) were obtained by density gradient centrifugation of venous phlebotomy specimens (14,16).

Diagnoses were prospectively established in IPF and COPD subjects by expert, specialized pulmonary clinicians, blinded to these experimental studies, who analyzed all clinical information, including medical histories and physical exams, PFTs, and laboratory studies that included serologic tests for conventional autoimmune syndromes, as well as expert rheumatology specialist evaluations, chest radiographs and computerized tomography scans. All IPF study subjects fulfilled consensus diagnostic criteria (1). None had clinical evidence or a past history of connective tissue diseases or other autoimmune diatheses (e.g., autoimmune hepatitis, ulcerative colitis, primary biliary cirrhosis), drug toxicities, malignancies, or occupational/environmental exposures associated with interstitial lung disease. Extensive histological evaluations of IPF pulmonary explants were performed in all specimens removed during therapeutic transplantations by expert lung pathologists who were unaware of these studies. All pulmonary explants or other lung biopsies from these IPF subjects had histological confirmation of usual interstitial pneumonia or end-stage fibrotic disease.

This study was approved by the University of Pittsburgh Institutional Review Board.

Lung Tissue Specimens: Methodologies for procurement and processing of pulmonary explant specimens have been detailed previously (14). In brief, surgically explanted lungs were obtained from IPF patients undergoing therapeutic pulmonary transplantations. Lung tissue (˜0.5-1 cm3) was dissected from the explants, embedded in optimal cutting temperature (OCT) media, frozen, and stored at −80° C. for later immunohistochemistry studies.

Water soluble protein extracts of lung explant specimens were also obtained by repeated freezing and thawing, as detailed previously (14). Lung extracts, protein fractions, and all other test antigens used in T-cell functional assays (HSP70, GRP78, TdT) were boiled for 20 minutes prior to use, to obviate nonspecific mitogen effects. Normal pulmonary specimens were similarly obtained from harvests of cadaveric lungs that were not used for transplantations. No lung specimen used in these studies had evidence of infection by clinical history, gross examination, microbiologic cultures, or histological evaluations.

Bronchoalveolar lavages (BAL) were obtained from lung explants by wedging sterile 5 mm plastic tubing in segmental bronchi of the middle lobe or lingula and infusing and withdrawing five successive 30 ml aliquots of PBS using a syringe. The BAL was centrifuged (400 g) to remove cells and particulates and sterile filtered (0.2 μm), before storage at −80°. Proteins in BAL were concentrated using 3 kDa centrifugation-size filters (Millipore, Bellerica, Mass.), and quantified by bicinchoninic acid (BCA) assay (Thermo Scientific, Rockfort, Ill.).

Three (3) OCT embedded lung specimens from patients who died from respiratory failure during acute IPF exacerbations were obtained from the Warm Autopsy Program of the University of Pittsburgh Tissue Bank for use in immunohistochemistry preparations.

Protein Fractionations: Aqueous lung extracts were separated by isoelectric focusing (IEF) into five fractions varying in pH from 3-10, using reagents, gels, and procedures supplied in a kit (ZOOM™ IEF Fractionator, Invitrogen, Carlsbad, Calif.). Proteins within each fraction were precipitated by acetone, desalted, concentrated by centrifugation, and quantified by BCA. Protein concentrations were adjusted in RPMI, sterile filtered, and boiled prior to use in T-cell proliferation assays.

Corroborative assays were conducted with another subsequent series of distinct pulmonary explant specimens using ion exchange chromatography (IEC) to fractionate lung extract proteins by pI. Proteins in buffered water (20 mM Tris, pH 8) that bound to a strong anion exchange column (HiTrap Q XL, GE Healthcare, Piscataway, N.J.) were eluted into three fractions by stepwise increments of buffer ionic strength (i.e., 0.33 M, 0.66 M, and 1.0 M NaCl). Proteins within these fractions were desalted, concentrated, quantified, and prepared for use in functional assays as described for IEC preparations.

Autoantibody Detection by Indirect Fluorescence: Plasma samples from IPF patients or normal subjects were diluted 1:40 in PBS, and incubated with fixed HEp-2 cells (Fluorescent HEp-2000 ANA, ImmunoConcepts, Sacramento, Calif.) for 30 min. After washing, IgG antibodies were detected using FITC-conjugated anti-human IgG antibody.

Humoral Autoantigen Discovery: IgG was isolated from pooled plasma of six IPF patients, already known to have autoantibodies to multiple K562 cell antigens on prior study (14), by adherence to protein A columns (HP SpinTrap, GE Healthcare). After extensive washing, the bound antibodies were covalently cross-linked to the protein A, following the manufacturer's protocol. Otherwise identical “preadsorption” columns were prepared using IgG from normal control plasma specimens.

Autoantigen sources for these immunoprecipitations were either aqueous protein extracts from normal lungs or K562 cell lysates, as described in the text. Both preparations were initially preadsorbed, to decrease nonspecific binding, by passing them thru the normal IgG-protein A columns. The eluants of these preadsorptions were then applied to the IPF patient IgG-protein A columns and extensively washed. The captured cell proteins (putative autoantigens) were then eluted by acidification, pH neutralized, and concentrated by centrifugal size-filtration.

These immunoprecipitation products were electrophoresed by two dimension 10.5% sodium dodecyl sulfate polyacrylamide gel electrophoresis (SDS-PAGE). Gels were imaged by Typhoon TRIO (GE Healthcare) and analyzed by Image QuantTL software (GE Healthcare). Individual proteins were harvested by spot picking (Ettan Spot Picker, GE Healthcare), trypsin digested, and peptides sequences identified by matrix-assisted laser desorption/ionization tandom time of flight mass spectrometry (MALDI-TOF/TOF) (Applied Biosystems, Carlsbad, Calif.).

Circulating Autoantibody Assays: Autoantibodies against HSP were detected in individual patient plasma by immunoblots. Recombinant HSP (rHSP70, rGRP78) were purchased from Prospec (Rehovot, Israel), and 250 ng was added to each lane of running gels (NuPage 4-12% Bis Tris, Invitrogen). After electrophoresis (90 minutes @130 V), the protein was transferred to 0.45 μm nitrocellulose membranes (Invitrogen). Gels were blocked with 5% dry milk in TTBS (50 mM Tris HCl [pH 7.4], 150 mM NaCl, 0.1% Tween 20). Individual lanes were separated by sectioning and incubated with subject sera (1:10 dilution in the blocking buffer) overnight at 4° C. The membrane strips were extensively washed in TTBS, and then incubated for 1 hour at room temperature with 1:8000 dilutions of chicken anti-human IgG conjugated to horseradish peroxidase (HRP) (Thermo Scientific). After another extensive washing, the HRP was detected on radiographic film after addition of Super Signal West Pico Chemiluminescent Substrate (Thermo Scientific).

Detection of HSP70 in BAL and Lung Extracts: Immunoblots were also used to detect HSP70 in clinical specimens. Individual aliquots of aqueous lung extracts (144 μg total protein) or BAL (36 μg total protein) were loaded in individual lanes of running gels, electrophoresed, and then transferred to membranes as described in the preceding section. HSP70 was detected using 1:1000 dilutions of mouse anti-human HSP 70 mAb (United States Biological, Swampscott, Mass.) in overnight incubations at 4° C. After extensive washing, membranes were incubated with chicken anti-mouse IgG-HRP (Santa Cruz Biotechnology, Santa Cruz, Calif.) at 1:4000 dilution, and subsequent steps and imaging were identical to that described previously.

Immunohistochemical Localization of HSP70 in Lung Tissues: Snap frozen OCT embedded lung tissues were freshly cut and fixed with cold acetone and 5% formalin mixture for 3 minutes. Tissues were blocked with Peroxidase Blocking Kit (Dako, Glostrup, Denmark) and then normal horse serum for 30 minutes. Sections were incubated with mouse anti-human HSP70 (Stressgen, Ann Arbor, Mich.) at 4° C. overnight. Following rinsing with PBS, the sections were incubated with biotinylated goat anti-mouse IgG (Vector Laboratories, Burlingame, Calif.) at room temperature for 1 hour, and then treated with AB Complex HRP (Vector Laboratories) for 30 min. Color was developed by adding ImmPACT DAB (Vector Laboratories) substrate for 1 minute. Images were captured with a photomicroscope (Zeiss, Thornwood, N.Y.) and scored (positive vs. negative) by two investigators blinded to subjects' clinical features and diagnoses.

Immunohistochemical Detection of IgG Immune Complexes and Fixed Complement in Lung Tissues: These methods have been described previously (16). In brief, frozen lung sections were cut and fixed in 2% PFA for 20 minutes. Sections were treated in 20 mM citrate buffer pH 6, at 95° C. for 10 minutes, blocked with normal serum for 30 minutes, and then incubated overnight with mouse (IgM) anti-human IgG (Serotec, Raleigh, N.C.) or mouse IgG2a anti-human C3 (Abgent, San Diego, Calif.) at 4° C. Tissues were rinsed and incubated with biotinylated goat anti-mouse IgM or anti-mouse IgG secondary antibody (Vector Laboratories) for one hour, and then rinsed with Tris buffer before adding AB Complex HRP (Dako) for 30 min. Color was developed by adding DAB substrate for 3-4 minutes. Microscopic images were taken in 6-9 randomly-selected fields from each specimen, and scored (positive vs. negative) by two investigators blinded to subjects' clinical features and diagnoses.

T-cell Functional Assays: These assays were performed using hilar lymph node (HLN) cells from lung explants (14) or PBMNC cells obtained by phlebotomies of ambulatory subjects.

HLN cells for T-cell autoantigen discovery assays had been previously stimulated by 11+1 d incubations in the presence of autologous lung protein extracts (100 μg/ml) and 10 U/ml IL-2, as described previously (14). Suspension and adherent HLN cells were then harvested (the latter by brief trypsin:EDTA treatment), pooled, washed, and replated at a density of 200,000 cells/well in 96 well plates. The cells were then cultured at 37° in 7% CO2 for five additional days in 200 μl of complete media (RPMI 1640 supplemented with 50 U/ml penicillin, 50 μg/ml streptomycin, 10 mM HEPES, 2 mM glutamine, and 10% heat-inactivated human AB serum) in the presence of boiled autologous lung extract protein fractions at final concentrations of 1 μg/ml.

PBMNC for proliferation and cytokine assays were used immediately after isolation, without prior antigen stimulation, but otherwise similarly cultured in the presence of no added protein (baseline controls), or the specified boiled antigens (i.e., rHSP70, rGRP78, TdT) at 1 μg/ml. TdT was purchased from EMD Chemicals, Gibbstown, N.J.

Proliferation was determined by DNA incorporation of either 3H-thymidine or bromodeoxyuridine (BrdU), as detailed elsewhere (14,16). In brief, 3H-thymidine was added (1 μCi) ˜18 hours prior to harvest, counts per minute [cpm]) were measured by beta emission counter in triplicate wells, and results averaged. BrdU incorporation was measured using reagents and protocols supplied in a kit (BD Bioscience, San Jose, Calif.). BrdU (10 uM) was added two days prior to harvest, whereupon the cells were stained with anti-CD4-APC mAb (BD Bioscience), permeabilized and fixed. The cells were subsequently incubated with anti-BrdU FITC and the percentage of proliferating cells (BrdU+) was determined among viable CD4 T-cells. Specific indices (SI) of proliferation were calculated as % of CD4 T-cells that incorporated BrdU in cultures with added antigen (rHSP70, rGRP78, or TdT) minus incorporation in concurrent unstimulated (baseline control) cultures.

Intracellular cytokine productions were determined by flow cytometry of PBMNCs cultured for one day with and without added antigens. The cells were stained with anti-CD4-APC mAb, and protein transport inhibitor (GolgiStop™, BD Bioscience) was added six hours prior to harvest. The PBMNC were then fixed and permeabilized, using reagents and products supplied in a kit (Cytofix/Cytoperm, BD Bioscience), and stained with PE-conjugated antibodies against either IL-4 or IFN-γ from the same manufacturer. Specific indices (SI) of cytokine production were calculated as % of CD4 T-cells that produced the cytokine in cultures with added antigen (HSP70, GRP78, or TdT) minus the production in concurrent unstimulated (baseline control) cultures.

Quantitations were performed on >10,000 live cells and analyzed using a BD FACSCalibur (BD Bioscience). Flow cytometry gates were set using control fluorochrome positive and negative PBMNC (including isotype controls), as detailed elsewhere (16).

HLA Allele Characterizations: HLA Class II alleles had already been delineated in a 51 of the IPF subjects in the course of other studies by using polymerase chain reactions with sequence specific primers (PCR-SSP) (19). Indications in this discovery cohort that DRβ1*11 prevalence may be under-represented in those patients with HSP70 autoantibodies were further examined by determining the presence or absence of DR β1*11 in banked DNA extracted from leukocytes of the remaining (validation) IPF cohort, again using PCR-SSP reagents and methods provided in a kit (Invitrogen).

Statistical Analysis: Two group comparisons of continuous variables were made by Mann-Whitney tests. Wileoxon signed rank tests were used to compare results of two or more assays in the same specimens, with corrections for multiple comparisons by Bonferroni. Dichotomous variables were analyzed by Chi-square, and odds ratios (OR and 95% confidence intervals (CI) determined by logistic regression. Survival analyses were performed using product-limit estimation, with comparisons by log-rank (Mantel-Cox). Hazard ratios (HR) and 95% CI of data that included censored observations were established by proportional hazard regression model. Analyses were conducted with StatView v5.0.1 (SAS Institute, Cary, N.C.). Alpha (p) values <0.05 after corrections for multiple comparisons were considered significant. Data are depicted as means+SE.

### 7.2. Results

Partial Characterization IPF T-cell Antigens: Previous investigations had shown that one or more antigens within aqueous extracts of IPF lungs induced proliferations of autologous CD4 T-cells (14).

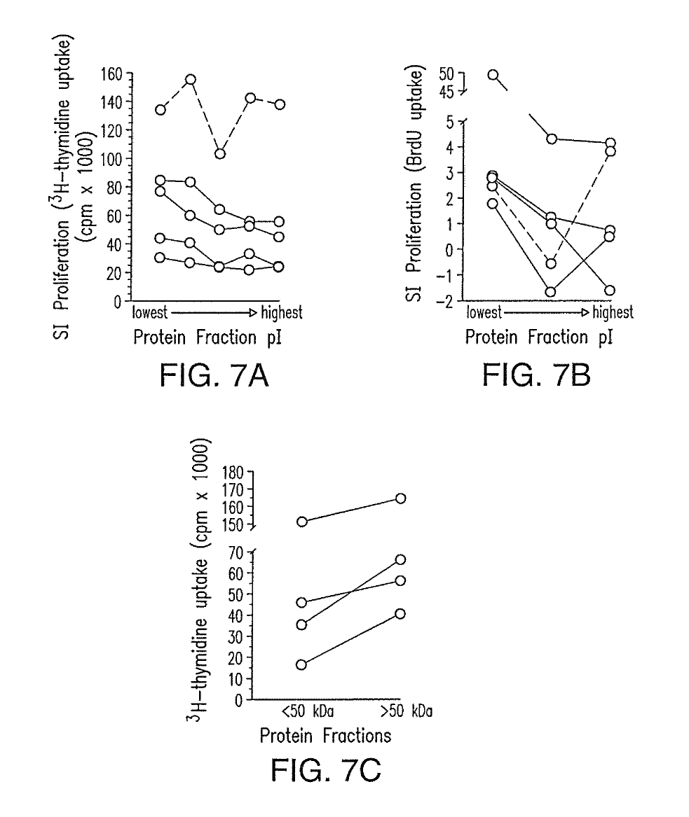

Interval efforts to identify dominant intrapulmonary IPF epitope(s) included partial protein fractionations of the complex lung explant preparations, and bioassays of these fractions for antigen enrichments, based on their ability to stimulate proliferation of autologous CD4 T-cells. The most acidic protein fractions (lowest pI) tended to be the most antigenic (FIGS. 7A and 7B). Additional segregations of proteins within low pI fractions by centrifugal size filtration further suggested the greatest antigenicity is among proteins >50 kDa (FIG. 7C).

Identification of Candidate Humoral Antigens: Given inherent difficulties with purification of T-cell antigens from complex (but limited) tissue extracts, and the interdependence of B-cell and T-cell antigen-specific responses (20), we hypothesized that circulating IgG antibodies isolated from IPF subjects could perhaps be used to affinity purify candidate autoantigens. This approach might thus allow us to first (and perhaps most productively) focus on further biologic study of those humoral autoantigens with physical characteristics that corresponded to the predominant CD4 T-cell antigen(s) (i.e., low pI and size >50 kDa).

Immunoprecipitates of pulmonary explant extracts, an obvious potential source of antigens (FIGS. 1A-1C) (14), using IPF patient-derived IgG, were obfuscated by high concentrations of nonspecific products that included abundant native immunoglobulins (3-5) and other, seemingly irrelevant background proteins (e.g., albumin).

However, IPF patients have diverse IgG autoantibodies with specificities against the proteins of many cell types (6-13), including HEp-2 (FIG. 7D-I) and K562 cells (14). Additional unpublished findings of the latter study also indicated that the presence of an autoantibody with specificity for a cryptic ˜70-80 kDa K562 cell antigen tended to have more rapid pulmonary function deterioration.

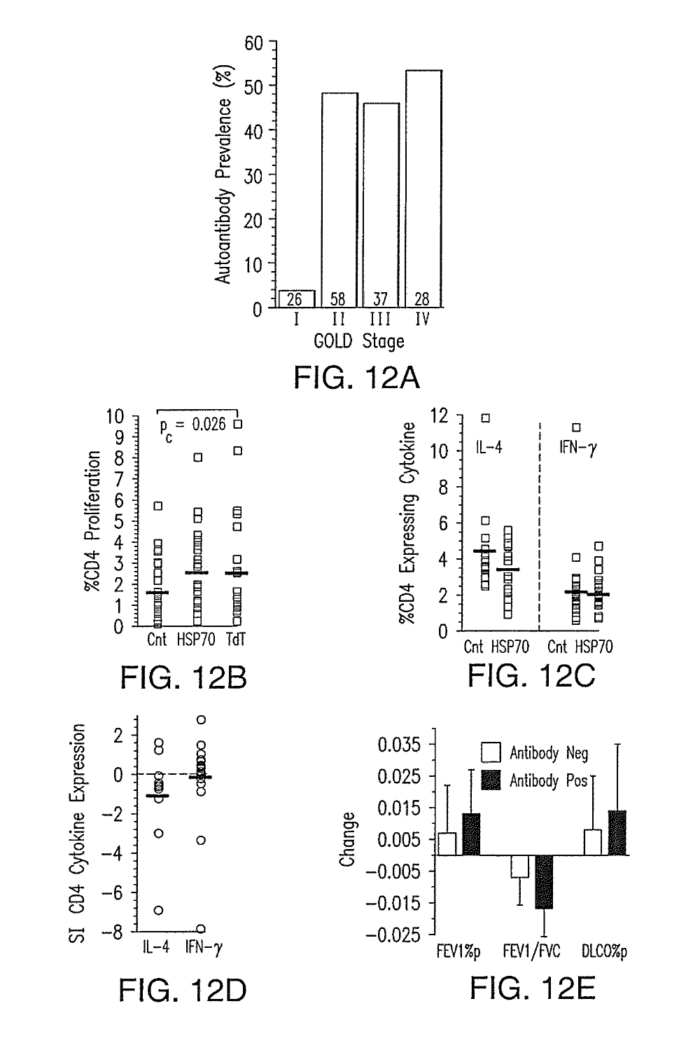

Plasma IgG from pooled IPF patients immunoprecipitated proteins from K562 cell lysates that were distinctly resolved by 2-D gel electrophoresis. Mass spectroscopy of the immunoprecipitated proteins with low pI and size ˜70-80 kDa (FIG. 13) identified two potential autoantigens on replicate assays: heat shock protein 70 (HSP70) and glucose regulated protein 78 (GRP78). HSP70 and GRP78 are both members of the HSP70 family of heat shock proteins (HSP), each have important biological functions, and they share extensive peptide sequence homology (28). Furthermore, both HSP70 and GRP78 have been identified as self-antigens in other autoimmune disorders (29-34). We also speculated a priori that concurrent study of the potential autoreactivities of both homologous HSP could provide useful comparative insights.

Circulating Autoantibodies to HSP70 and GRP78: Plasma specimens from IPF patients and healthy controls were assayed for autoantibodies against these HSP. Demographic characteristics of the IPF subjects are detailed in TABLE 6. Eight other subjects initially diagnosed with IPF were excluded from analyses here because they had abnormal conventional serologic tests (i.e., positive ANA, SSA/Ro, SSB, or Th/To autoantibodies), although they did not exhibit corresponding clinical manifestations (i.e., symptoms or signs of systemic lupus erythematosus [SLE], Sjogren's syndrome, or scleroderma, respectively).

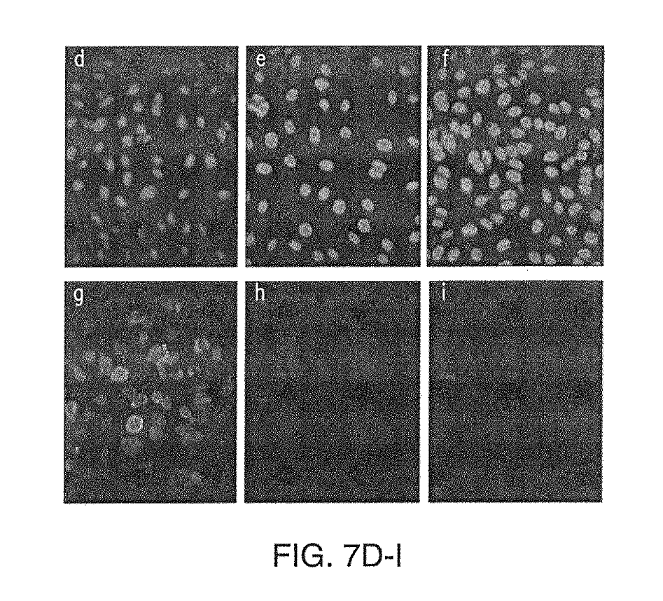

The normal controls were comparable in terms of age (65+1 years old) and gender distribution (67% males). Similarly, 54% of the healthy controls were former smokers (>5 pack years). Circulating IgG autoantibodies against both HSP were significantly more prevalent in the IPF patients than among the controls (FIG. 8A).

We assumed that if the presence of autoantibodies with avidities for HSP was solely the result of nonspecific, globally increased immunoglobulin productions and, as such, an epiphenomenal concomitant of underlying inflammation (e.g., an acute phase response), humoral responses against these highly homologous proteins would likely be synchronous. However, there were considerable discordances of the autoreactivities, as most patients who had autoantibodies against one HSP did not have IgG with specificities for the other (FIG. 8B).

Two of the eight IPF subjects excluded from analyses here because of their positive (albeit clinically occult) conventional autoimmune serologic tests also had anti-HSP70 IgG autoantibodies. One of these two, and two of the other excluded subjects, also had circulating anti-GRP78 autoantibodies.

HSP Expression in Lungs: Both HSP70 and GRP78 were present in all IPF pulmonary specimens evaluated here by immunoblot, but less frequently detected, and usually in lesser amounts, in the equivalent preparations from normal lung explants (FIG. 8C).

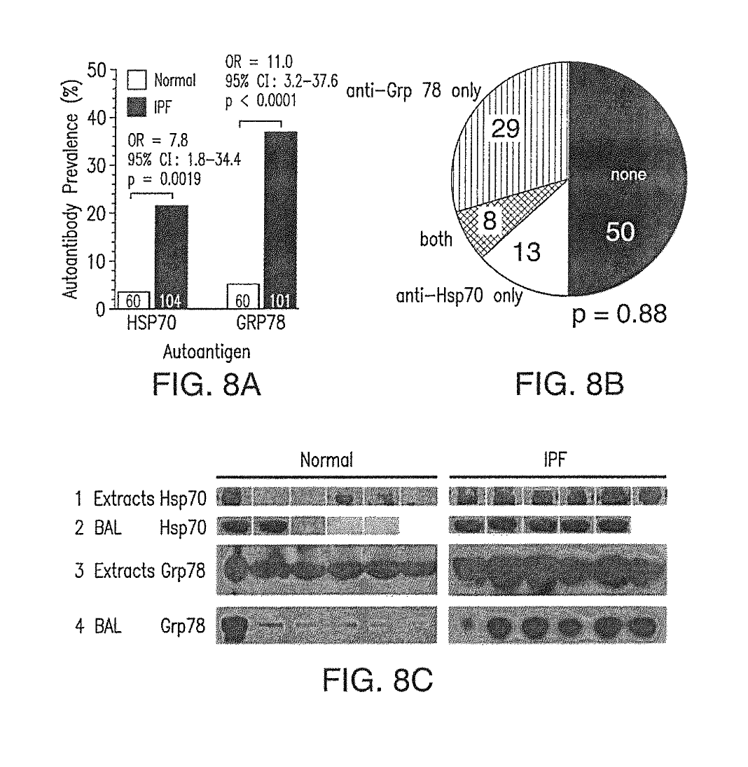

IPF CD4 T-cell Autoreactivity to HSP: Circulating CD4 T-cells from IPF subjects were more consistently stimulated to proliferate by HSP70 than by either GRP78 or tetanus toxoid (TdT) (FIGS. 9A and 9B). We had previously shown that production of profibrotic IL-4 tended to be increased among CD4 T-cells from IPF patients, and associated with disease severity (14). CD4 T-cells from the majority of IPF patients increased their IL-4 production in cultures supplemented with HSP70, unlike effects seen with GRP78 (FIGS. 9C and 9D).

Clinical Correlates of Heat Shock Protein Autoreactivity: We hypothesized that if anti-HSP autoantibodies play a role in IPF pathogenesis their presence could be associated with clinical manifestations.

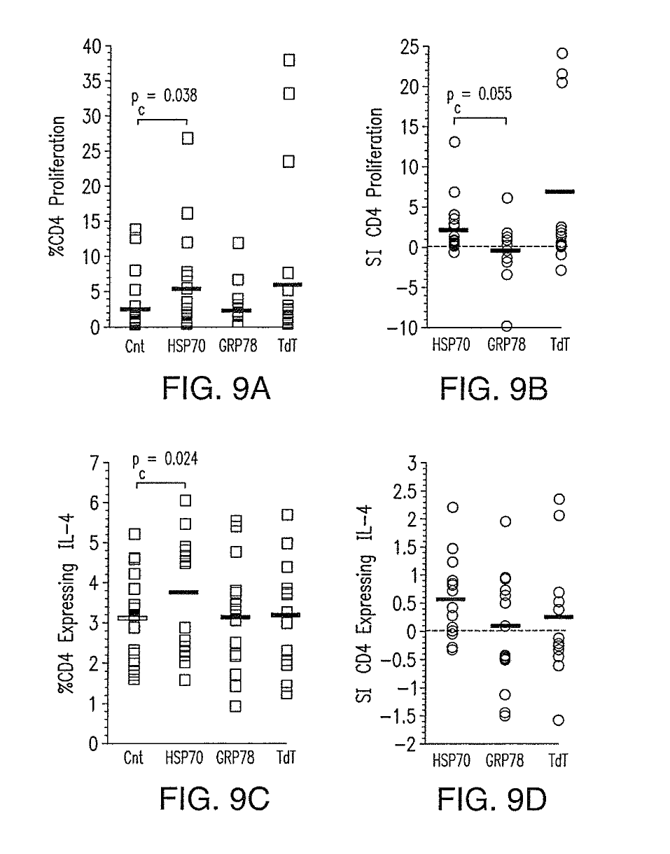

There were no significant demographic or pulmonary function differences at the time of specimen acquisitions between the IPF patients who had anti-HSP70 autoantibodies (anti-HSP70 Positive) or those who were negative for this autoimmune response (anti-HSP70 Negative), nor between anti-GRP78 Positive or Negative subjects (TABLE 6). Although the numbers of patients who had subsequent pulmonary function tests (PFTs) were diminished due to interval attritions (i.e., deaths, lung transplantations, or becoming too ill to perform this testing), there were nonetheless greater decrements of pulmonary function in the anti-HSP70 Positive patients, compared to patients who did not have this autoantibody (FIG. 10A). PFTs were performed 5.5+0.9 and 6.2+0.3 months after their plasma specimens were obtained from the anti-HSP70 Positive and Negative subjects, respectively (p=0.34).

However, there was no apparent association between subsequent changes of pulmonary function and GRP78 humoral autoreactivity (FIG. 10B).

Progression of lung dysfunction in IPF patients almost inevitably results in either lung transplantation or death of those afflicted (1). These dichotomous outcomes are not true independent events, however, in that lung transplantation ostensibly diminishes or delays death of IPF patients, albeit by no means invariably, and this surgical procedure is associated with unique complications and an inherent mortality.

Survival analyses, with lung transplantations tabulated as censored events (i.e., the end of observations), showed one-year mortality was significantly greater among the IPF patients who had anti-HSP70 autoantibodies (FIG. 10C). Overt respiratory failure accounted for eight of the nine deaths (89%) that occurred in the anti-HSP70 Positive cohort and six of the 12 deaths (50%) in the patients who were anti-HSP70 Negative. Aside from one case of fatal pulmonary emboli among the latter, the other unattributable deaths in both subpopulations occurred at home or in hospices, without detailed, proximate pre-mortem evaluations or autopsies.

A limited number of IPF subjects, most of whom were recent referrals to our clinics, were taking immunosuppressive drugs at the time of specimen acquisitions (TABLE 6). Post hoc analyses limited to those patients not taking immunosuppressants showed anti-HSP70 autoantibodies were present in 23% of these subjects. One-year survival was 53+13% vs. 87+4% for the un-medicated anti-HSP70 Positive and un-medicated anti-HSP70 Negative patients, respectively (p=0.008).

To exclude potential cryptic biasing by skewed selections of subjects for lung transplantations, despite double blinding between the clinicians and laboratory investigators, post hoc survival analyses were performed in which the lung transplantations were also considered major-adverse-events equivalent to death. The one-year major-adverse-event-free survival of IPF patients who had anti-HSP70 autoantibodies remained less than that of anti-HSP70 Negative subjects (36+10% vs. 67+5%, respectively, p<0.01).

There was no apparent association between the presence of GRP78 autoantibodies and mortality among the IPF patients (FIG. 10D).

Stability of Anti-HSP70 Autoantibody Responses: Although this initial investigation was designed as a cross-sectional study, we were fortuitously able to collect and analyze replicate plasma specimens from some longer-living subjects, in order to access the “stability” of their anti-HSP70 autoimmune responses. Two surviving IPF patients with anti-HSP70 IgG at their initial specimen assays remained positive for these autoantibodies at replicate determinations 13 and 38 months later, and both have since died. Sixteen (16) other IPF patients who were anti-HSP70 negative on their first evaluation remained negative at replicate assays performed after intervals of 11+2 months. Two IPF patients who were initially negative for anti-HSP IgG developed these antibodies after intervals of 12 and 30 months, and one of these died 5 months later.

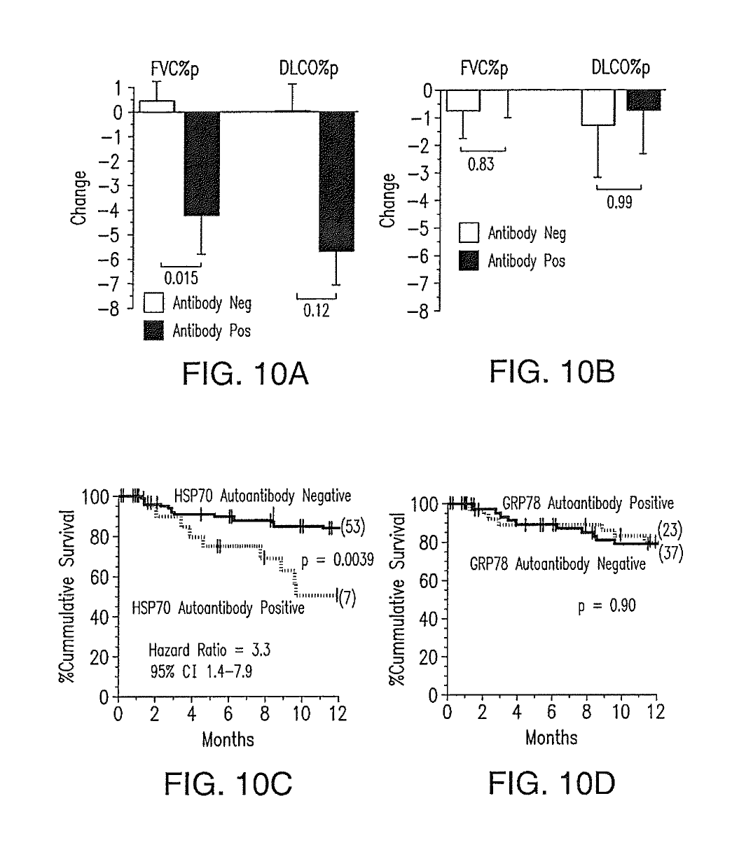

Immunohistochemistry: In situ expression of HSP70 was evident in all six IPF lung explants procured during therapeutic transplantations (FIG. 11A) and all three warm autopsy specimens from patients who died from respiratory failure during acute IPF exacerbations (FIG. 11E). HSP70 expression was especially prominent in distal airway and alveolar epithelium, macrophages, to lesser extents in endothelial cells, and appeared to be increased relative to expression of this autoantigen in normal lung specimens (FIG. 11I).

Autoantibody-mediated immunopathogenesis within a target organ is evidenced by findings of antigen-antibody (immune) complexes and fixed complement (35-37), whereas these processes are infrequent in normal lung explants (38). Similarly, immune complexes were not seen in five of the six normal lung explants examined here (FIG. 5J), and were only equivocally present in one other. Fixed complement (C3) was not evident in any of the normal lung explants (FIG. 11K). In contrast, IgG complexes were seen in four of six (67%) IPF explants (FIG. 11B) and equivocally present in another. Immune complexes were prominent in the lungs of all three patients who died during acute IPF exacerbations (FIG. 11F). C3 deposits were found in three of the six IPF lung transplantation explants (FIG. 11C) and two of the three necropsy specimens from the acute IPF exacerbation cohort (FIG. 11G).

HLA Allele Bias: Antigen-specific autoimmune responses are often associated with over- and/or under-representations of specific HLA alleles (19). Fifty-one (51) of the IPF patients here had full HLA Class II allele typing performed during the course of earlier studies (19). The initial comparison of allele prevalences (the proportion of subjects with one or more allele copies) in this discovery cohort indicated a possible under-representation of DRβ1*11 among those subjects with anti-HSP70 antibodies compared to patients who did not have this autoimmune response (0 vs. 16.2%, p=0.11) (TABLE 7). These findings were replicated by DRβ1*11 typing of the remaining IPF subjects, which confirmed this HLA allele seems to be protective for the development of humoral HSP70 autoreactivity (0 vs. 17.3%, for the anti-HSP70 autoantibody positive and negative subjects, respectively, p=0.036).

HSP70 Autoreactivity in Chronic Obstructive Pulmonary Disease (COPD): The presence of autoreactivity to HSP70 among patients with varying seventies of COPD was also ascertained.

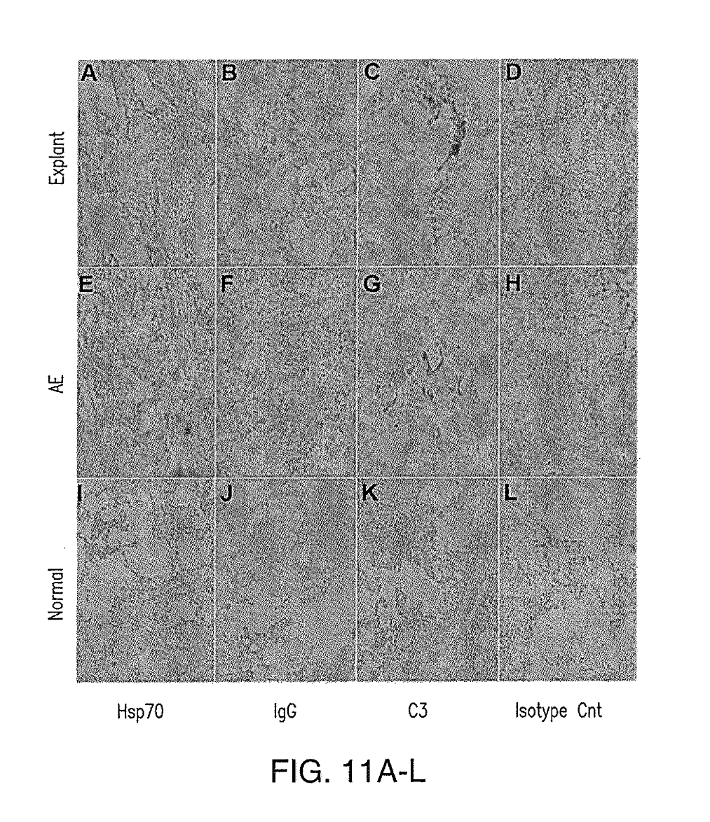

Anti-HSP70 autoantibodies were only present in one patient with mild COPD, but were seen in approximately 50% of the subjects with moderate-severe obstructive lung disease (FIG. 12A). However, HSP70 was not a significant antigen for circulating T-cells of COPD patients (FIGS. 12B-D). Furthermore, anti-HSP70 autoantibody responses were not associated with other demographic or clinical manifestations (TABLE 8), or with subsequent changes in pulmonary function over the next two years (FIG. 12E). None of the COPD subjects died during the two year observation period of this study.

### 7.3 Discussion

These data show that circulating anti-HSP 70 IgG autoantibodies are more prevalent in IPF patients than among healthy, normal subjects (FIG. 8A). Nonspecific, polyclonal production of immunoglobulins with clinically irrelevant specificities, as a secondary consequence of other immunologic processes, often results in only transient presence of particular autoantibodies. However, the apparent stability of humoral HSP70 autoreactivity in IPF patients (i.e., the persistence of positive or negative autoantibody assays among individual subjects) is consistent with features of a chronic immune response driven by a distinct, specific autoantigen (37). An association with a distinct HLA allele (either an over- or under-representation) is another typifying characteristic of antigen-specific autoimmune responses (19), and the presence of DRβ1*11 appears to protect from production of anti-HSP70 autoantibodies in IPF patients. The biologic plausibility of an autoimmune response is also enhanced by finding the autoantigen is actually present within the diseased target organ, and HSP70 is abundant in IPF lungs (FIGS. 8D, 11A, 11E). Deposits of antigen-antibody complexes and fixed complement, de facto evidence of antibody-mediated pathogenic processes (35,36), are also common in these specimens (FIGS. 11B, 11C, 11F, 11G). Moreover, the production of IgG with avidity for protein antigens (and autoantigens) by B-lymphocytes is dependent on the provision of facultative “help” by CD4 T-cells that share antigen receptor specificity for these particular peptides (20). Concomitant CD4 T-cell reactivity to HSP70 is frequent in IPF patients (FIG. 9) and unique to this disease population (FIG. 12). Collectively, these findings establish the presence of HSP70-specific autoreactivity in a cohort of IPF patients (35,37).

The robust associations between the presence of anti-HSP70 autoantibodies and disease manifestations (FIG. 10) further suggest this immune response is involved in the pathogenesis of IPF progression (35-37). Antibodies to HSP70 occurred in only 21% of the IPF study subjects, so obviously these findings do not implicate humoral anti-HSP70 autoreactivity as the sole “cause” of IPF. Nonetheless, the presence of HSP70 autoantibodies was disproportionately associated with subsequent pulmonary function deterioration (FIG. 10A) and nearly half of the deaths in the study population (FIG. 10C). Multiple other autoantibodies, with both known and unknown specificities, have also been described in IPF patients (5-14), others are likely to be discovered in incremental studies, and it is possible (if not probable) that additional antigen-specific autoimmune responses could also be involved in the pathogenesis of this disease (35,37).

Immunologic reactivity to various HSP, including HSP70, has been described in other autoimmune syndromes (29-34,39). Although the mechanism by which these autoreactivities develop is uncertain, the process is not necessarily due to a seminal, de novo loss of self-tolerance for these ubiquitous proteins. HSP expression is up-regulated by a wide variety of cellular injuries and stresses, including proximate immunological injuries originally directed at other antigens. High concentrations of HSP in or near ongoing inflammatory foci or other injuries may be conducive for promotion of autoreactivity against these proteins per se by epitope spread (35). In addition, or alternatively, microbial HSP have been implicated as inciting factors for development of autoreactivity to the stress response molecules of their human hosts, presumably a result of epitope cross-reactivity (“mimicry”) between the highly conserved determinants of these orthologous molecules (35, 37, 39). Once initiated, autoimmune responses tend to be self-perpetuating and often intractable, despite removal or cessation of the inciting processes, since the target self-antigens that fuel these responses are continually renewed. In cases of autoreactivity to HSP, ongoing inflammation [or other injury] could presumably result in increased expression of the antigenic stress response molecules, potentially creating a positive feedback loop that could accelerate disease progression and clinical exacerbations (21).

HSP70 binds to several cell surface receptors (28,29), and by analogy to other autoimmune disorders (35,37), IgG autoantibodies with specificity for these membrane-bound self-antigens could cause tissue injury by diverse mechanisms, including induction of NK cell-mediated cytotoxicity (38), or by fixing complement (36). Either process can directly kill antibody-bound target cells, while the latter can also activate and recruit neutrophils to the inflammatory foci, and both alveolar epithelial cell apoptosis and intrapulmonary neutrophilia are prominent features of IPF (1,21). Autoantibodies are also capable of gaining access to intracellular antigens via endocytosis (40) where they can exert varied deleterious effects on target cell functions (9, 33, 34). Incremental studies to define the pathogenic mechanisms of the anti-HSP70 IgG from IPF patients are an ongoing area of investigation in our laboratories. Unraveling the specific causes and processes by which autoantibody responses exert their characteristic tissue injuries, however, is often a lengthy and difficult endeavor. For example, despite appreciation of antinuclear autoantibodies (ANA) in SLE for more than 50 years, neither the etiology nor immunopathophysiologic mechanisms of tissue damage in this syndrome are fully understood (41).

Nonetheless, the likely pathogenecity of anti-HSP70 immune responses in IMF is also supported by findings here that HSP70 is an antigen for the CD4 T-cells of many patients (FIG. 9). Under normal circumstances, and by various mechanisms, T-cells are inert to anatomically accessible autologous proteins, and thereby they do not initiate or fuel autoimmune injuries. However, autoantigen-activated CD4 T-cells can trigger and sustain immune conflagrations by a wide variety of directly injurious actions, as well as by production of mediators that activate and/or recruit cascades of downstream effectors (42). Given the range and magnitude of pathogenic effects of antigen-activated T-cells, it is difficult to envision that lymphocyte proliferation and profibrotic cytokine production (FIG. 9) induced by an abundant self protein (FIGS. 1C, 5A, 5E) is likely to be a benign or epiphenomenal process. The present findings may wholly or partially account for previous observations of antigen-driven oligoclonal CD4 T-cell expansions (14,15), and clinically-correlated accumulations of end-differentiated, dysregulated, and highly pathogenic lymphocytes among IPF patients (16).

In addition to their presence in other autoimmune syndromes (29-32), autoantibodies to HSP70 were also present in COPD subjects (FIG. 12A), a clinically distinctive chronic lung disease in which autoimmune processes have been implicated (38,43). In contrast to IPF patients, however, autoantibodies to HSP70 in COPD subjects were not accompanied by appreciable CD4 T-cell reactivity to this autoantigen (FIG. 12), nor associated with clinical manifestations (FIG. 12F, TABLE 8). Various aspects of fundamental immunological processes are often shared among clinically distinct syndromes, and relatively few autoantibody specificities are absolutely limited to a single, particular disease (35, 37, 44, 45). Despite the promiscuity of many immunologic responses, however, assays to determine the presence of defined autoantibodies within specific patient subpopulations, in turn delineated by their shared, underlying clinical features, have relevance for understandings of pathogenic mechanisms, and are frequently useful in the medical care of individual patients. As one of many examples, ANA assays are widely used in the diagnosis and management of patients with SLE, but ANA are also present in patients with most other autoimmune/rheumatologic disorders, some drug reactions, chronic active hepatitis, and numerous infections (45). In distinction to findings in IPF patients, the data here suggest that anti-HSP70 autoantibody production is epiphenomenal in COPD patients, analogous to ANA positivity in many inflammatory conditions other than SLE (45). Thus, the present findings do not support the use of anti-HSP70 autoantibody assays in the differential diagnosis of chronic lung disease. However, other, more conventional evaluations (e.g., medical histories, physical exams, PFTs, and radiographs) are readily capable of distinguishing patients with IPF from those with COPD (1).

In summary, these data show that autoantibodies with specificities for HSP70 are uniquely associated in IPF subjects with subsequent pulmonary function deterioration and excess mortality. This autoantigen is abundant in IPF lungs, along with intrapulmonary immune complex and complement depositions that are highly suggestive of antibody-mediated pathogenesis. Specific T-cell reactivity to this autoantigen is also common in IPF patients. These data implicate autoimmune processes, and autoreactivity to HSP70 in particular, in the progression of disease among an IPF cohort.

TABLE 6
Demographic and Clinical Characteristics of IPF Subjects with
Autoantibody Studies

| | Anti- | Anti- | Anti- | Anti- | |
|---|---|---|---|---|---|
| | HSP70 | HSP70 | GRP78 | GRP78 | |
| | Negative | Positive | Negative* | Positive* | Aggregate |
| n | 82 | 22 | 64 | 37 | 104 |
| Age (yrs) | 70 ± 1 | 69 ± 2 | 70 ± 1 | 69 ± 1 | 70 ± 1 |
| Gender (% | 73 | 68 | 72 | 70 | 71 |
| male) | | | | | |
| Lung Biopsy | 55 | 45 | 58 | 49 | 54 |
| (%) | | | | | |
| FVC % | 62.5 ± 2.0 | 60.2 ± 4.6 | 60.6 ± 2.4 | 64.9 + 3.1 | 62.0 ± 1.9 |
| predicted | | | | | |
| DL_{CO} % | 48.5 ± 2.1 | 45.5 ± 4.9 | 49.9 ± 2.5 | 45.1 + 2.7 | 47.9 ± 1.9 |
| predicted | | | | | |
| Smoke (≧5 | 51 | 57 | 50 | 56 | 54 |
| pack-yr)(%) | | | | | |
| Immuno- | 21 | 14 | 22 | 16 | 19 |
| meds (%) | | | | | |

Negative and Positive denote IPF subjects who do not have or do have circulating anti-HSP70 or GRP78 IgG autoantibodies, respectively. *GRP78 autoantibody assays could not be performed in three IPF specimens due to insufficient amounts of plasma. Immuno-meds denote subjects taking any single agent or various permutations of prednisone (5-20 mg/day), azathioprine, interferon-gamma, mycophenolate, or tacrolimus. None of the intergroup differences were significant.

TABLE 7
HLA Class II allele prevalence in the discovery cohort.

| | Anti-HSP70 Negative | Anti-HSP70 Positive | p value |
|---|---|---|---|
| n | 37 | 14 | |
| DQβ1* Alleles | | | |
| 02 | 40.5 | 21.4 | 0.215 |
| 03 | 48.6 | 64.3 | 0.32 |
| 04 | 2.7 | 0 | NA |
| 05 | 29.7 | 21.4 | 0.55 |
| 06 | 51.4 | 50.0 | 0.93 |
| DRβ1* Alleles | | | |
| 01 | 16.2 | 14.3 | 0.87 |
| 03 | 27.0 | 28.6 | 0.91 |
| 04 | 13.5 | 21.4 | 0.49 |
| 07 | 16.2 | 14.3 | 0.87 |
| 08 | 2.7 | 7.1 | 0.47 |
| 09 | 5.4 | 0 | NA |
| 10 | 5.4 | 0 | NA |
| 11 | 16.2 | 0 | 0.11 |
| 13 | 24.3 | 35.7 | 0.42 |
| 14 | 0 | 7.1 | NA |
| 15 | 32.4 | 42.9 | 0.49 |
| 16 | 10.8 | 7.1 | 0.69 |
| 18 | 5.4 | 0 | 0.38 |

HLA Class II allele prevalences (the percentages of subjects with one or more copies of the allele) in 51 of IPF patients here had been previously determined in prior study (19). The greatest apparent intergroup difference of allele prevalences among anti-HSP autoantibody negative and positive subjects was with DRβ1*11 (bold). This finding was subsequently validated by DRβ1*11 typing the remaining IPF subjects. Very rare alleles (i.e., those only present in one subject) were not further analyzed here (NA),

TABLE 8
Demographic and Clinical Characteristics of COPD Subjects with
Autoantibody Studies

| | Anti-HSP70 | Anti-HSP70 |
|---|---|---|
| | Negative | Positive |
| n | 63 | 60 |
| Age (yrs) | 66 ± 1 | 65 ± 1 |
| Gender (% male) | 62 | 55 |
| Still Smoking (%) | 26 | 30 |
| FEV₁ (% predieted) | 49 ± 3 | 49 ± 3 |
| FEV₁/FVC | 0.44 ± 0.02 | 0.46 ± 0.02 |
| DL_{CO} (% predicted) | 47 ± 2 | 47 ± 3 |
| Emphysema Score | 3.0 ± 0.2 | 2.7 ± 0.2 |
| Six minute walk (feet) | 386 ± 23 | 276 ± 25 |

Negative and Positive denote COPD subjects who do not have or do have circulating anti-HSP70 autoantibodies. GOLD classifications are detailed elsewhere (46). GOLD 1 subjects are not described here given the small number of those subjects who were anti-HSP70 Positive (FIG. 12A). FEV₁=forced expiratory volume in the first second of expiration. Emphysema scores are based on blinded, expert radiologist quantifications (48). None of the intergroup differences were significant.

### 7.4. REFERENCES

1. American Thoracic Society. Idiopathic pulmonary fibrosis: diagnosis and treatment. International consensus statement. American Thoracic Society (ATS), and the European Respiratory Society (ERS). Am J Respir Crit Care Med. 161, 646-664 (2000).
2. D. A. Campbell, L. W. Poulter, G. Janossy, R. M. du Bois, Immunohistological analysis of lung tissue from patients with cryptogenic fibrosing alveolitis suggesting local expression of immune hypersensitivity. Thorax. 40, 405-11 (1985).
3. J. Marchal-Somme, Y. Uzunhan, S. Marchand-Adam, D. Valeyre, V. Soumelis, B. Crestani, P. Soler, Cutting edge: non-proliferating mature immune cells form a novel type of organizing lymphoid structure in idiopathic pulmonary fibrosis. J. Immunol. 176, 5735-5739 (2006).
4. F. Zuo, N. Kaminski, E. Eugui, J. Allard, Z. Hakhini, A. Ben-Dor, L. Lollini, D. Morris, Y. Kim, B. DeLustro, D. Sheppard, A. Pardo, M. Selman, R. A. Heller, Gene expression analysis reveals matrilysin as a key regulator of pulmonary fibrosis in mice and humans. Proc. Natl. Acad. Sci. USA. 99, 6292-97 (2002).
5, P. P. Dall Aglio, A. Pesci, G. Bertorelli, E. Brianti, S. Scarpa, Study of immune complexes in broncholaveolar lavage fluids. Respiration. 54, 36-41 (1988).
6. N. Dobashi, J. Fujita, M. Murata, Y. Ohtsuki, I. Yamadori, T. Yoshinouchi, R. Ueda, S. Bandoh, T. Kamei, M. Nishioka, T. Ishida, J. Takahara, Elevation of anti-cytokeratin 18 antibody and circulating cytokeratin 18: anti-cytokeratin 18 antibody immune complexes in sera of patients with idiopathic pulmonary fibrosis. Lung. 178, 171-9 (2000).
7. B. Grigolo, I. Mazzetti, R. M. Borzi, H. D. Hickson, M. Fabbri, L. Fasano, R. Meliconi, A. Facchini, Mapping of topoisomerase II alpha epitopes recognized by autoantibodies in idiopathic pulmonary fibrosis. Clin Exp Immunol. 114, 339-46 (1998).
8. Y. Yang, J. Fujita, S. Bandho, Y. Ohtsuki, I. Yamadori, T. Yoshinouchi, T. Ishida, Detection of antivimentin antibody in sera of patients with idiopathic pulmonary fibrosis and non-specific interstitial pneumonia. Clin Exp Immunol. 128, 169-74 (2002).
9. W. H. H. Wallace, S. M. Howie, Upregulation of tenascin and TGF-□ production in a type II alveolar epithelial cell line by antibody against a pulmonary auto-antigen. J. Pathol. 195, 251-6 (2001).
10. C. M. Magro, W. J. Waldman, D. A. Knight, J. N. Allen, T. Nadasdy, G. E. Frambach, P. Ross, C. B. Marsh, Idiopathic pulmonary fibrosis related to endothelial injury and antiendothelial cell antibodies. Hum Immunol. 67, 284-297 (2006).
11. F. Ogushi, K. Tani, T. Endo, H. Tada, T. Kawano, T. Asano, L. Huang, Y. Ohmoto, M. Muraguchi, H. Moriguchi, S. Sone, Autoantibodies to IL-1□ in sera from rapidly progressive idiopathic pulmonary fibrosis. J Med Invest. 48, 181-9 (2001).
12. K. Kurosu, Y. Takiguchi, O. Okada, N. Yumoto, S. Sakao, Y. Tada, Y. Kasahara, N. Tanabe, K. Tatsumi, M. Weiden, W. N. Rom, T. Kuriyama, Identification of annexin 1 as a novel autoantigen in acute exacerbation of idiopathic pulmonary fibrosis. J Immunol. 181, 756-767 (2008).
13. C. Taille, S. Grootenboer-Mignot, C. Boursier, L. Michel, M. P. Debray, J. Fagart, L. Barrientos, A. Mailleux, N. Cigna, F. Tubach, J. Marchal-Somme, P. Soler, S. Chollet-Martin, B. Crestani. Identification of Periplakin as a New Target for Autoreactivity in Idiopathic Pulmonary Fibrosis. Am J Respir Crit Care Med. October 8. [Epub ahead of print] (2010).
14. C. A. Feghali-Bostwick, C. G. Tsai, V. G. Valentine, S. Kantrow, M. W. Stoner, J. M. Pilewski, A. S. Gadgil, M. P. George, K. F. Gibson, A. M. Choi, N. Kaminski, Y. Zhang, S. R. Duncan, Cellular and humoral autoreactivity in idiopathic pulmonary fibrosis. J Immunol. 179, 2592-9 (2007).
15. A. Shimizudani, H. Murata, H. Keino, S. Kojo, H. Nakamura, Y. Morishima, T. Sakamoto, M. Ohtsuka, K. Sekisawa, M. Sumida, T. Sumida, T. Matsuoka, Conserved CDR 3 region of T cell receptor BV gene in lymphocytes from bronchoalveolar lavage fluid of patients with idiopathic pulmonary fibrosis. Clin Exp Immunol. 129, 140-149 (2002).
16. S. R. Gilani, L. J. Vuga, K. O. Lindell, K. F. Gibson, J. Xue, E. K. Lindsay, N. Kaminski, V. G. Valentine, M. P. George, C. Steele, S. R. Duncan, CD28 down-regulation on circulating CD4 T-cells is associated with poor prognoses of patients with idiopathic pulmonary fibrosis. PLoS One. 5:e8959 (2010).
17. I. O. Rosas, P. Ren, N. A. Avila, C. K. Chow, T. J. Franks, W. D. Travis, J. P. McCoy Jr., R. M. May, H. P. Wu, D. M. Nguyen, M. Arcos-Burgos, S. D. MacDonald, B. R. Gochuico, Early interstitial lung disease in familial pulmonary fibrosis. Am J Resp Crit Care Med. 176, 698-705 (2007).
18. I. Kotslanidis, E. Nakou, I. Bouchliou, A. Tzouvelekis, E. Spanoudakis, P. Steiropoulos, I. Sotiriou, V. Aidinis, D. Margaritis, C. Tsatalas, D. Bouros, Global impairment of CD4+CD25+FoxP3+regulatory T cells in idiopathic pulmonary fibrosis. Am J Resp Crit Care Med. 179, 1121-1130 (2009).
19. J. Xue, B. R. Gochuico, A. S. Alawad, C. A. Feghali-Bostwick, I. Noth, S. D. Nathan, G. D. Rosen, I. O. Rosas, S. Dacic, I. Ocak, C. R. Fuhrman, K. T. Cuneco, M. A. Smith, S. S. Jacobs, A. Zeevi, P. A. Morel, J. M. Pilewski, V. G. Valentine, K. F. Gibson, N. Kaminski, F. C. Sciurba, Y. Zhang, S. R. Duncan, The HLA Class II allele DRB1*1501 is over-represented in patients with idiopathic pulmonary fibrosis. PLoS One. 6:e14715 (2011)
20. D. C. Parker, T-cell dependent B-cell activation. Annu Rev Immunol. 11, 331-340 (2006).
21. H. R. Collard, B. B. Moore, K. R. Flaherty, K. K. Brown, R. J. Kaner, T. E. King Jr., J. A. Lasky, J. E. Loyd, I. Noth, M. A. Olman, G. Raghu, J Roman, J. H. Ryu, D. A. Zisman, G. W. Hunninghake, T. V. Colby, J. J. Egan, D. M. Hansell, T. Johkoh, N. Kaminski, D. S. Kim, Ko Y. Kondoh, D. A. Lynch, J. M. Quernheim, J. L. Myers, A. G. Nicholson, M. Selman, G. B. Toews, A. U. Wells, F. J. Martinez, with the Idiopathic Pulmonary Fibrosis Clinical Research Network Investigators. Acute exacerbations of idiopathic pulmonary fibrosis. Am J Respir Crit Care Med. 176, 636-643 (2007).
22. S. B. Erickson, S. B. Kurtz, J. V. Donadio, K. E. Holley, C. B. Wilson, A. A. Pineda, Use of combined plasmapharesis and immunosuppression in the treatment of Goodpasture's syndrome. Mayo Clin Proc. 54, 714-720 (1979).
23. M. Sem, O. Molberg, M. B. Lund, J. T. Gran, Rituximab treatment of the anti-synthetase syndrome: a retrospective case series. Rheumatology. (Oxford). 48, 968-971 (2009).
24. T. Martinu, D. N. Howell, S. M. Palmer, Acute cellular rejection and humoral sensitization in lung transplant recipients. Semin Resp Crit Care Med. 31, 179-188 (2010).
25. R. Borie, M. P. Debray, C. Laine, M. Aubier, B. Crestani, Rituximab therapy in autoimmune pulmonary alveolar proteinosis. Eur Respir J. 33, (6). 1503-6. (2009).
26. G. Bussine, L. Mouthon, Interstitial lung disease in systemic sclerosis. Autoimmun Rev. 2010 Sep. 21. [Epub ahead of print]
27. J. H. Stone, P. A. Merkel, R. Spiera, P. Seo, C. A. Langford, G. S. Hoffman, C. G. Kallenberg, E. W. St Clair, A. Turkiewicz, N. K. Tchao, L. Webber, L. Ding, L. P. Sejismundo, K. Mieras, D. Weitzenkamp, D. Ikle, V. Seyfert-Margolis, M. Mueller, P. Brunetta, N. B. Allen, F. C. Fervenza, D. Geetha, K. A. Keogh, E. Y. Kissin, P. A. Monach, T. Peikert, C. Stegeman, S. R. Ytterberg, U. Specks, RAVE-ITN Research Group. Rituximab versus cyclophosphamide for ANCA-associated vasculitis. N Engl J Med. 15, 363(3):22′-32 (2010).
28. M. Wisniewska, T. Karlberg, L. Lehtio, I. Johansson, T. Kotenyova, M. Moche, H. Schüller. Crystal structures of the ATPase domains of four human Hsp70 isoforms: HSPA1L/Hsp70-hom, HSPA2/Hsp70-2, HSPA6/Hsp70a, and HSPA5/BiP/GRP78. PLoS One. 5, e8625 (2010)
29. Z. Prohaszka, Chaperones as part of immune networks. Adv Exp Med Biol. 594, 159-66 (2007).
30. F. Tahiri, F. Le Naour, S. Huguet, R. Lai-Kuen, D. Samuel, C. Johanet, B. Saubamea, V. Tricottet, J. C. Duclos-Vallee, E. Ballot, Identification of plasma membrane autoantigens in autoimmune hepatitis type 1 using a proteomics tool. Hepatology. 47, 937-48 (2008).
31. D. Zlacka, P. Vavrincova, T. T. Hien Nguyen, I. Hromadnikova, Frequency of anti-HSP60, -65 and -70 antibodies in sera of patients with juvenile idiopathic arthritis. J. Autoimmun. 27, 81-8 (2006).
32. R. Abulafia-Lapid, D. Gillis, O. Yosef, H. Atlan, I. R. Cohen I R, T cells and autoantibodies to human HSP70 in Type 1 diabetes in children. J Autoimmun. 20, 313-321 (2003).
33. M. Gonzalez-Grwonow, M. Guchacovich, C. Llanos, C. Urzua, G. Gawdi, S. V. Pizzo, Prostate cancer cell proliferation in vitro is modulated by antibodies against glucose-regulated protein 78 isolated from patient serum. Cancer Res. 66, 11424-11431 (2006).
34. M. C. Lu, N. S. Lai, H. C. Yu, H. B. Huang, S. C. Hsieh, C. L. Yu, Anti-citrullinated protein antibodies bind surface-expressed citrullinated Grp78 on monocyte/macrophages and stimulate tumor necrosis factor alpha production. Arthritis Rheum. 62, 1213-23 (2010).
35. P. Marrack, J. Kappler, B. L. Kotzi, Autoimmune disease: why and where it occurs. Nat Immunol. 7, 899-905 (2001).
36. T. N. Mayadas, G. C. Tsokos, N. Tsuboi. Mechanisms of immune complex-mediated neutrophil recruitment and tissue injury. Circulation. 120, 2012-2024 (2009).
37. Y. Naparstek, P. H. Plotz, The role of autoantibodies in autoimmune disease. Annu Rev Immunol. 11, 79-104 (1993).
38. C. A. Feghali-Bostwick, A. S. Gadgil, L. E. Otterbein, J. M. Pilewski, M. W. Stoner, E. Csizmadia, Y. Zhang, F. C. Sciurba, S. R. Duncan, Autoantibodies in patients with chronic obstructive pulmonary disease. Am J Resp Critical Care Med. 177, 156-163 (2008).
39. S. Hoshida, M. Nishion, J. Tanouchi, T. Kishimoto, Y. Yamad, Acute Chlamydia pneumonia infection with heat shock protein 60-related response in patients with acute coronary syndromes. Atherosclerosis. 183, 109-112 (2005).
40. J. Y. Jang, J. G. Jeong, H. R. Jun, S. C. Lee, J. S. Kim, Y. S. Kim, M. H. Kwon, A nucleic acid-hydrolyzing antibody penetrates into cells via caveolae-mediated endocytosis, localizes in the cytosol and exhibits cytotoxicity. Cell Mol Life Sci. 66, (11-12):1985-97 (2009).
41. P. E. Lipsky, Systemic lupus erythematosus: an autoimmune disease of B cell hyperactivity. Nat Immunol. 2, 764-766 (2001).
42. C. Monaco, E. Andereakos, S. Kiriakidis, M. Feldman, C. Paleolog, T-cell-mediated signaling in immune, inflammatory and angiogenic processes: the cascade of events leading to inflammatory diseases. Curr Drug Targets Inflamm Allergy. 3, 35-42 (2004).
43. M. G. Cosio, M. Saetta, A. Agusti, Immunologic aspects of chronic obstructive pulmonary disease. N Engl J Med. 360, 2445-54 (2009).
44. V. D. Steen, D. L. Powell, T. A. Medsger, Clinical correlations and prognosis based on serum autoantibodies in patients with systemic sclerosis. Arthritis Rheum. 31, 196-203 (1988).
45. D. H. Solomon, A. J. Kavanaugh, P. H. Schur, American College of Rheumatology Ad Hoc Committee on Immunologic Testing Guidelines. Evidence-based guidelines for the use of immunologic tests: antinuclear antibody testing. Arthritis Rheum. 47(4), 434-44 (2002).
46. Global Initiative for Chronic Obstructive Lung Disease. Global strategy for the diagnosis, management, and prevention of chronic obstructive pulmonary disease. www.goldcopd.com. Date last assessed Mar. 10, 2010
47. D. O. Wilson, J. L. Weissfeld, A. Balkan, J. G. Schragin, C. R. Fuhrman, S. N. Fisher, J. Wilson, J. K. Leader, J. M. Siegfried, S. D. Shapiro, F. C. Sciurba. Association of radiographic emphysema and airflow obstruction with lung cancer. Am J Resp Crit Care Med. 178, 738-744 (2008).
48. X. Zhu, A. S. Gadgil, R. Givelber, M. P. George, M. W. Stoner, F. C. Sciurba, S. R. Duncan. Peripheral T-cell functions correlate with the severity of chronic obstructive pulmonary disease. J. Immunol 2009; 182:3270-3277.

### 8. Example

### Decreased Survival of Idiopathic Pulmonary Fibrosis Patients with Heat Shock Protein 70 Autoreactivity

Background: Although the pathogenesis of idiopathic pulmonary fibrosis (IPF) remains elusive, abnormal immune responses are often evident among afflicted patients. We hypothesized autoimmune responses may be involved in idiopathic pulmonary fibrosis (IPF) pathogenesis.

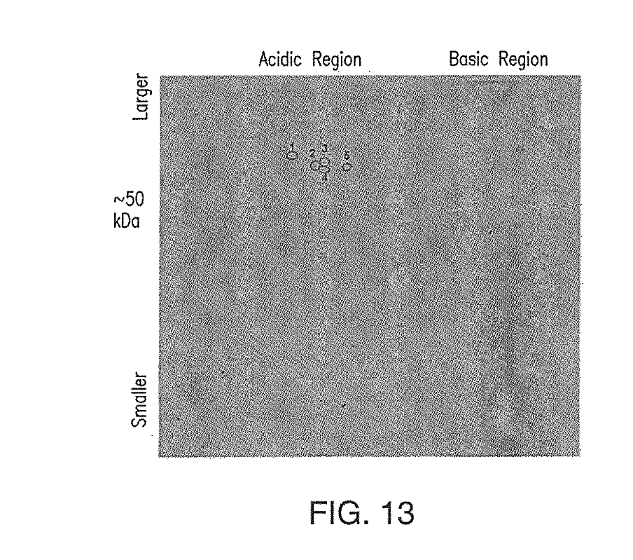

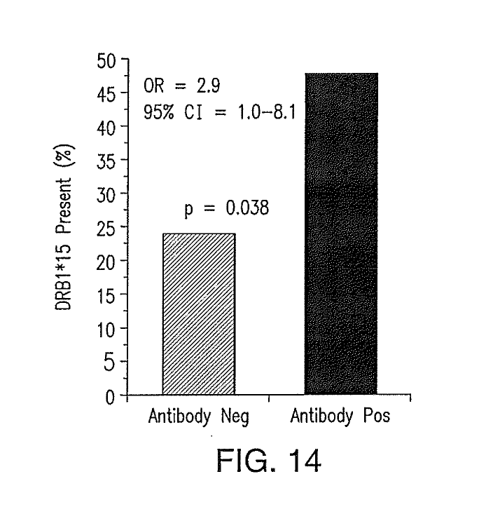

Measurement and main Results: Putative autoantigens of IPF were identified by immunoprecipitation of cell lysates, using IgG-derived from IPF patients, 2-D gels, spot picking, and mass spectroscopy. Heat shock proteins were among the most frequently isolated potential autoantigens in these patients. Validation study showed that IgG autoantibodies against heat shock protein 70 (Hsp70) were detected by immunoblots in 24% of 88 IPF patients vs. 3% of 60 age- and gender-matched healthy controls (p=0.0007). IPF lungs ubiquitously expressed Hsp70 per in situ IHC and immunoblots of BAL and lung extracts. Intrapulmonary immune complexes and fixed complement were also prevalent in IPF lung specimens. Circulating IPF CD4 T-cells became activated, proliferated, and produced IL-4 when co-cultured with Hsp70 in vitro. There were no demographic or pulmonary function differences (at the time of specimen collections) between IPF patients with and without anti-Hsp70 autoantibodies. The IPF subpopulation with anti-Hsp70 autoantibodies, however, had over-representations of HLA-DRB1*1501 (FIG. 14), greater pulmonary function deterioration over the next 6 months, and significantly decreased one-year survival (FIG. 15). Hsp70 polymorphisms also associated with patient survival among two distinct IPF cohorts (p=0.007). Autoantibodies against highly homologous glucose regulated protein 78 were also common in IPF patients (34%), but were discordant with anti-Hsp70 responses, unassociated with T-cell autoreactivity, HLA allele frequencies, or clinical manifestations. Hsp70 autoantibodies were also present in 41% of chronic obstructive pulmonary disease patients (n=149), but seemed similarly epiphenomenal in this cohort, and unassociated with current or future pulmonary function, HLA allele frequency perturbations, or T-cell autoreactivity.

Conclusions: Diverse autoreactivities of variable significance are common in patients with chronic lung diseases. However, unique findings in IPF patients of humoral and cellular Hsp70 autoreactivity, presences of this autoantigen and prototypic immunopathogenic processes in diseased lungs, a HLA allele bias, and strong associations with clinical manifestations suggest this antigen-specific autoimmune response is singularly involved in progression of this disease.

### 9. Example

### Hsp70 Single Nucleotide Polymorphisms (SNPS) in Idiopathic Pulmonary Fibrosis

Given the presence of HSP70-specific autoreactivity in IPF patients, and the strong associations between the presence of this autoimmune response and lung disease manifestations, we hypothesized that genetic variants of the autoantigen could perhaps have important influences on development of this immunologic response. HSP70 is encoded by three proximate genomic sequences in the HLA Class III region. SNPs within each of these genes are known, although the functional correlates of the respective genotypes have not yet been defined.

We hypothesized a priori that HPS70 polymorphisms could influence the development of autoimmunity in IPF subjects and/or survival of these patients by two general mechanisms: First, a HSP70 SNP could possibly affect the cellular production of this autoantigen, either during basal conditions or in conditions of stress. Although many complex factors influence the development of autoimmunity, a general truism is that increased amounts (or accessibility) of the antigen, particularly in proximity to ongoing inflammation, is conducive to promotion of these responses. Second, even single amino acid substitutions can profoundly alter antigen recognition and responses by effects on protein secondary and tertiary structures (conformations). Two of the SNPs we studied do not result in changes of protein primary structures, whereas the other, the SNP for HSP70-hom (see below), encodes alleles with distinct peptide sequences.

### Genotyping of the HSP70 Polymorphisms

Three SNPs were analyzed for HSP70 family genes. The rs1061581 of HSP70A1B was genotyped using PCR-based restriction fragment length polymorphism (RFLP) analysis. A 1117 bp DNA fragment was amplified by PCR using primers 5′-CAT CGA CTT CTA CAC GTC CA-3′ (SEQ. ID. NO: 1) and 5′-CAA AGT CCT TGA GTC CCA AC-3′ (SEQ. ID. NO: 2) in the presence of the MasterAmp PCR buffer G (Epicentre, Madison, Wis.). The PCR products were digested with Pst I which differentially digest the rs1061581G allele to generate two fragments with 184 bp and 933 bp in size. Genotypes were determined based on band patterns of an agarose gel electrophoresis analysis of the digested PCR products. The rs1043618 of HSP70A1A and rs2227956 of HSP70A1L (HSP70-hom) were analyzed using Taqman SNP primer/probe set C_{—}11917510_{—}10 and C_{—}25630755_{—}10, respectively, and 7900 HT DNA analyzer (Applied Biosystems, Foster City, Calif.).

### Clinical Correlates of HSP70-2 Gene SNPs

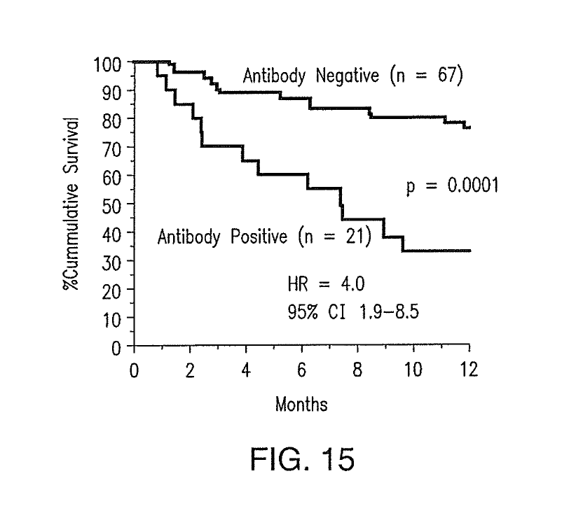

Product-limit analyses showed the two-year survival of the 21 IPF patients at the U. Pittsburgh who had the HSP70-2 GG genotype (rs1061581) was greater (86+9%) that that of the 96 subjects with either of the other two genotypes at this locus (either AA or AG) (60+6%) (p=0.03). To validate these findings, these SNPs were prospectively determined in a cohort of 35 IPF patients recruited at the NIH. The prevalence of the rs1061581 GG genotype in this replication cohort (20%) was near identical to that of the U. Pgh. discovery population (18%). IPF subjects among the NIH validation cohort with the GG genotype also had a two-year survival advantage (100% vs. 72+9%). Survival curves of the aggregate IPF subjects (U. Pgh.+NIH) are depicted in FIG. 16.

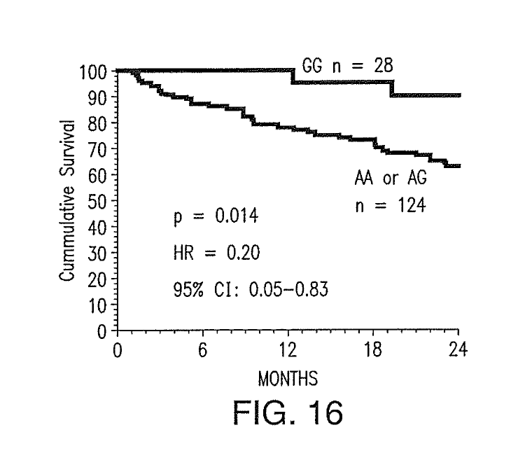

The mechanism(s) that accounts for the survival advantage of patients with the rs1061581 GG genotype is currently a subject of ongoing investigation in our laboratories. The observation cannot simply be attributed to differences of epitope differences conferred by HSP70 peptide sequences per se, since the proteins encoded by these various SNP are homologous. Results of an initial study indicate the GG genotype is associated with paradoxically decreased production of HSP70 in response to heat shock (FIG. 17). If confirmed in larger, ongoing studies, the survival advantage conferred by the GG genotype could plausibly be attributable to decreased production of HSP70 bp cells in proximity to or within inflammatory foci.

### Autoimmune Correlates of Hsp70-Hom Snps

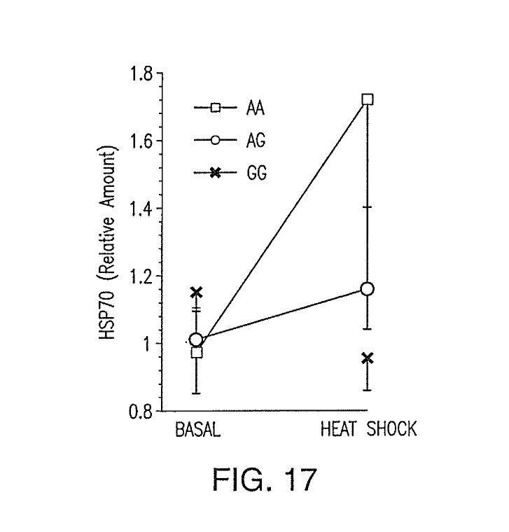

The presence of autoantibodies against HSP70 is highly associated with subsequent pulmonary function deterioration and mortality of IPF patients. An analysis of HSP70 SNPs has shown that the AA genotype associated with a nonhomologus protein variant of HSP70-hom rs2227956 is protective for development of this clinically-significant antigen-specific autoimmune response (FIG. 18).

In addition to the possibility that this AA genotype could alter production of the autoantigen (see FIG. 17), the peptide sequence encoded by this allele could possibly be less immunogenic. Alternatively, or in addition, the protective effect of this AA genotype could possibly be an indirect consequence of the often strong and nonrandom linkage disequilibrium (LD) between HSP70 genotypes and HLA allele polymorphisms. Abnormal frequencies of specific HLA alleles (either over- or under-representations) are a frequent finding of antigen-specific autoimmune responses, and often related to the specific epitope binding motifs of distinct HLA molecules. HLA DRB1*11 is under-represented in IPF patients with anti-HSP70 antibody responses. In turn, IPF subjects with the AA genotype of HSP70-hom seemingly have a ˜two-fold increased prevalence of DRB1*11 (17.6%) compared to patients with the other HSP70-hom genotypes (8.5%), although this intergroup difference does not reach statistical significance in this small population (p=0.15).

### 10. Example

### The Association of Autoimmunity with Emphysema and Osteoporosis

### 10.1. Materials and Methods

Subjects: Subjects were recruited in the context of an ongoing COPD SCCOR. Antibody studies and bone mineral density measures (DEXA) were established in a total of 267 subjects. Ninety-nine (99) of the subjects had no evidence of airflow obstruction (COPD) on PFTs (smoke controls [CS]). The remaining subjects had variable severities of COPD. All subjects fulfilled stringent diagnostic criteria (31). Diagnoses are prospectively established by expert clinicians, blinded to experimental study results, who analyze all clinical information, including medical histories, physical exams, pulmonary function tests (PFTs), evaluations by validated instruments (MMRC and St. George Questionnaires; 32,33) laboratory studies, chest radiographs and CT scans. Radiographic emphysema is scored by a single expert (and blinded) radiologist on a validated scale from 0-5: 0 denotes no emphysema, with higher point scores proportionate to emphysema severity (11). All study subjects (CS and COPD) have smoking histories (>10 pack years) and none has evidence or a history of autoimmune or connective tissue diseases, drug toxicities, or occupational/environmental exposures associated with lung disease. Laboratory investigators are blinded to subject identities and clinical manifestations.

In addition, healthy controls without smoking histories (Cnt) were recruited by solicitation from hospital personnel. Cnt were comparable in terms of age and gender distribution to the CS and COPD study subjects.

Mann-Whitney tests were used for comparisons of continuous or ordered variables, and Wilcoxon tests were used for paired values. Dichotomous variables were compared by chi-square, and odds ratios (OR) and 95% confidence intervals (CI) established by logistic regression. Continuous data here are depicted as means±SEM, and p values are delineated.

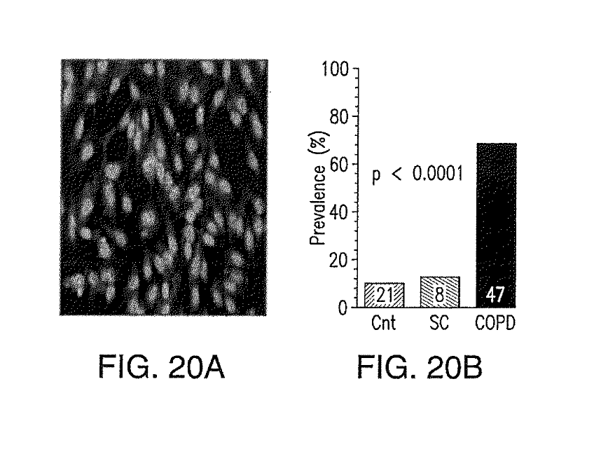

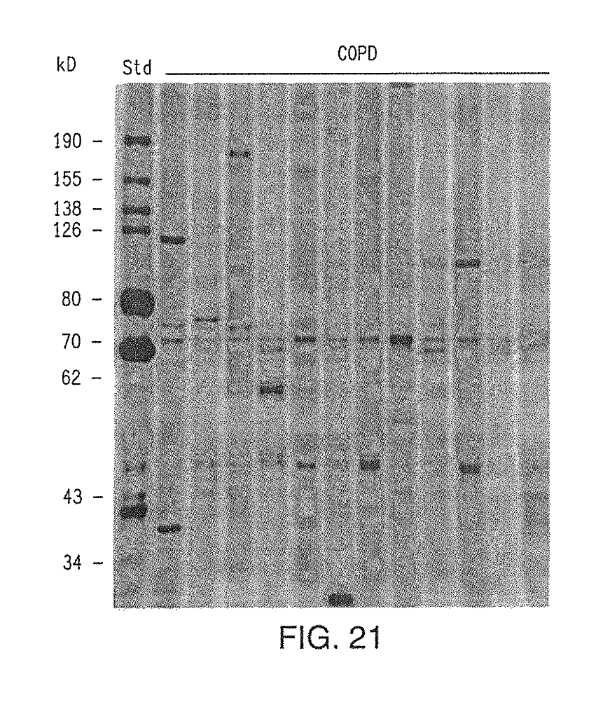

COPD Humoral Autoantigen Discovery: In order to identify specific autoantigens among subjects with smoking-associated lung disease, we immunoprecipitated cell lysate proteins with patient-derived IgG autoantibodies. IgG was isolated from pooled sera of six GOLD 3-4 COPD patients (known to have anti-epithelial autoantibodies on prior study; 26) by adherence to protein A columns (HP SpinTrap, GE Healthcare). These IgG were covalently cross-linked to the protein A following the manufacturer's protocol. K562 cell lysates (per cell sonication) were preadsorbed with normal IgG bound to protein A, and then putative autoantigens among these eluants were captured on the COPD IgG-protein A columns. We used K562 cell lysates (and protein A) because we knew these preparations identified numerous autoantigens of COPD patients (FIG. 21). The COPD IgG-bound cell lysate proteins (putative autoantigens) were eluted by acidification, pH neutralized, concentrated by centrifugal size-filtration (Millipore), and electrophoresed on 2-D gels (FIG. 22). Individual proteins were harvested by spot picking, trypsin digested, and sequenced by MALDI-TOF MS/MS (mass spectrometry [MS]).

Anti-GRP78 Autoantibody Measures: Plasma specimens were obtained by centrifugation of anti-coagulated blood from subjects, and screened for autoantibodies to recombinant GRP78 (rGRP78, Prospec, Rehovot, Israel) by immunoblots. Results of these studies are dichotomous (positive or negative) and highly specific. rGRP78 (200 ng) was loaded onto gels, electrophoresed, membrane transferred, and individual membrane lanes were cut out and incubated with 1:20 dilutions of individual plasma specimens. Chicken anti-human IgG-HRP (1:8000) was used as the secondary antibody. Blots are scored by two investigators blinded to subject identities and characteristics.

GRP78 Expression: GRP78 was detected in lung specimens using methods described previously (26).

Bone Mineral Density: DEXA scans were obtained and interpreted as described previously (10,34).

T-cell Studies: Specified antigens, as recombinant proteins, were added at 1 μg/ml final concentration to peripheral blood mononuclear cells (PBMNC) isolated from venous blood specimens. All antigens were boiled for 20 minutes to obviate nonspecific mitogens effects. Proliferation of CD4 T-cells among the PBMNC was determined by BrdU incorporation, as detailed elsewhere (36).

### 10.2. Results and Discussion

Subjects: Characteristics of the study subpopulations with smoking histories are detailed in TABLE 9.

TABLE 9

| | | Age | | | | | | Emphysema |
|---|---|---|---|---|---|---|---|---|
| | n | (yr) | % male** | PY | FEV₁ % p* | FEV₁/FVC* | DLCO % p* | (%)* |
| SC | 99 | 67 ± 1 | 53 | 62 ± 3 | 98 ± 1 | 77 ± 1 | 78 ± 2 | 43 |
| Gold 1 | 31 | 68 ± 1 | 77 | 71 ± 7 | 91 ± 2 | 64 ± 1 | 74 ± 2 | 71 |
| Gold 2 | 94 | 68 ± 1 | 50 | 60 ± 3 | 67 ± 1 | 55 ± 1 | 60 ± 2 | 78 |
| Gold 3 | 43 | 64 ± 1 | 70 | 67 ± 6 | 39 ± 1 | 39 ± 1 | 43 ± 2 | 90 |

SC=controls with extensive smoking histories, but normal pulmonary function tests; PY=pack years cigarette smoking *p<0.001, **p<0.02

Autoantigen Discovery: We have performed repeated IP isolations of cell lysate proteins using COPD patient IgG, with good concordance between replicate isolations. To date, 19 putative autoantigens have been identified in these gels (and many spots have not yet been “picked”) and biologic validations are ongoing.

The most extensively studied product of these IP assays to date is glucose regulated protein (GRP78). This 78 kDa heat shock protein (Hsp) was of particular interest, given it's multiple cellular functions and known association with autoimmunity (37-39). GRP78 is an endoplasmic reticulum (ER) chaperone and key element of the unfolded protein response (URL) that is critical for folding, maturation and transport of polypeptides and proteins. GRP78 expression is up-regulated by oxidative and various other URL stresses, including viral infections (40) ozone exposure, and cigarette smoke (41,42). Cell surface GRP78 is a co-receptor for viruses, mediates endocytosis (43), and also transduces pro-proliferation (e.g., cancer promoting) and anti-apoptosis signals (44,45).

Many Hsp molecules, including GRP78, are self-antigens of diverse autoimmune syndromes, probably because of their intimate association with antigen processing, and their up-regulation by stresses, including proximate infections or other inflammation (46). In particular, GRP78 is a major T-cell and B-cell autoantigen of rheumatoid arthritis (RA) (38), and anti-GRP78 autoantibodies also develop in many patients with cancer (37). Anti-GRP78 autoantibodies isolated from RA patients have been shown to increase NF KB activation and TNF-α production of a macrophage cell line (38).

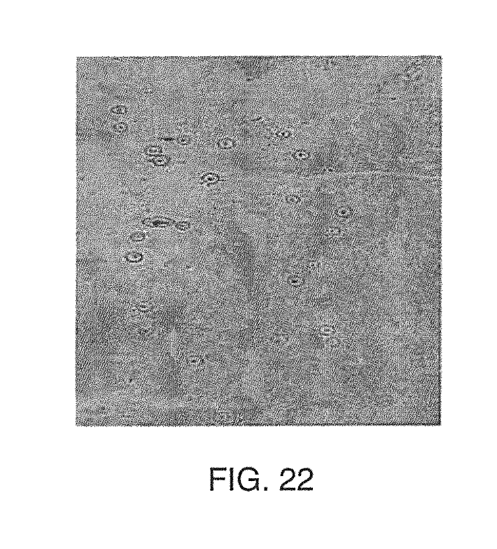

Antibodies to GRP78: Anti-GRP78 autoantibodies were present in 8% of normal subjects, which compares favorably with detections of other autoantibodies (e.g., antinuclear antibodies [ANA]) in ˜10% of healthy volunteers). Moreover, the prevalence of anti-GRP78 was comparatively increased in SC, and further increased with more severe PFT abnormalities (FIG. 23).

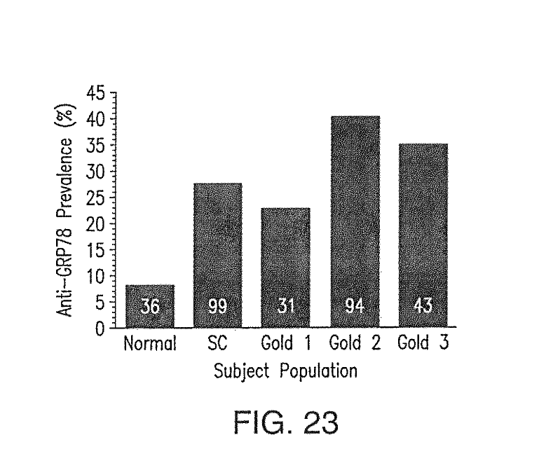

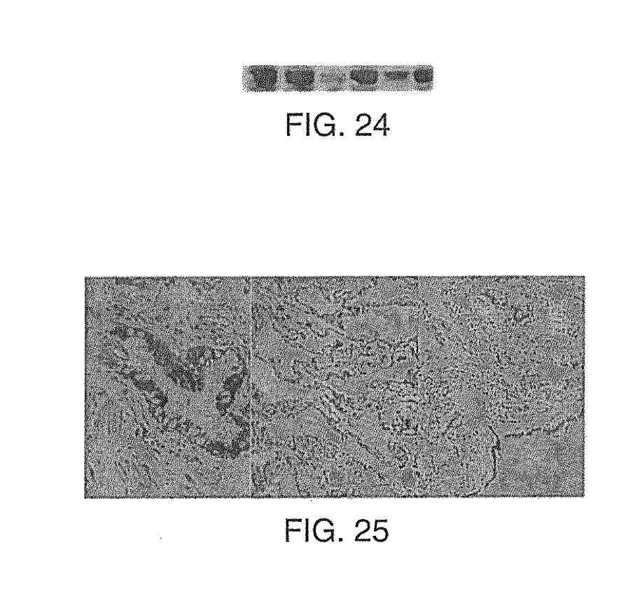

GRP78 in Lung Specimens: The credibility of GRP78 as an autoantigen would be enhanced by finding this protein is expressed in diseased lungs (15,16). GRP78 was detected in bronchoalveolar (BAL) fluid immunoblots of all 6 COPD lung explant tested to date, using anti-human GRP78 monoclonal antibodies (mAb) (FIG. 24). More importantly, immunohistochemistry (IHC) showed GRP78 is present in situ in six additional COPD lung explant specimens (distinct from the BAL specimens) (FIG. 25). We have analyzed 2 normal lung explants (removed from cadavers during organ harvests, but not used as donor organs due to size or ABO incompatibilities) by IHC (47), and it appears that GRP78 expression is comparatively diminished in the normal lungs.

GRP78 is also a T-cell Antigen of COPD: T-cells initiate and fuel adaptive immune responses, including production of IgG autoantibodies (48). T-cell processes are germane to findings of IgG autoantibodies because isotype switch from IgM to IgG with avidity for proteins is absolutely dependent on facultative help from the CD4 T-cells that have specificity for those particular antigens (49). Given this critical T-cell B-cell interdependence, findings that a particular suspect self-protein is an antigen for both lymphocyte subpopulations increases the likelihood this is a genuine autoimmune response. Furthermore, T-cells are readily capable of causing disease independently (or in concert) with autoantibody effects (48). We recently started testing responses of circulating T-cells from COPD subjects to GRP78 and other candidate antigens. Unlike non-cell type specific measures of proliferation (³H-thymidine incorporation) or IFN-γ production (ELISpot) in admixed cell cultures, which can be confounded by endotoxin stimulation of monocytes, macrophages and B-cells, we use flow cytometry to specifically define proliferation (BrdU incorporation) and intracellular cytokine production within T-cells (36, 47, 50) (which are not directly activated by endotoxin). GRP78 is a T-cell autoantigen of COPD patients (FIG. 8).

Clinical Correlates of Anti-GRP78 Autoantibodies: The only significant difference between subjects with and without anti-GRP78 autoreactivity was a female predominance among the former (TABLE 10). Most autoimmune syndromes are over-represented among females.

TABLE 10

| | | Age | | | | | |
|---|---|---|---|---|---|---|---|
| | n | (yr) | % male* | FEV1 % p | FEV1/FVC | DLCO % p | PY |
| Antibody | 87 | 67 ± 1 | 43 | 78 ± 2 | 0.60 ± 0.02 | 64 ± 2 | 59 ± 3 |
| Pos | | | | | | | |
| Antibody | 180 | 67 ± 1 | 63 | 74 ± 1 | 0.63 ± 0.01 | 67 ± 2 | 65 ± 2 |
| Neg | | | | | | | |

Antibody Pos and Antibody Neg denote SC subjects with and without anti-GRP78 autoantibodies, respectively. % p=percentages of predicted values; PY=pack years cigarette smoking; *p=0.009,

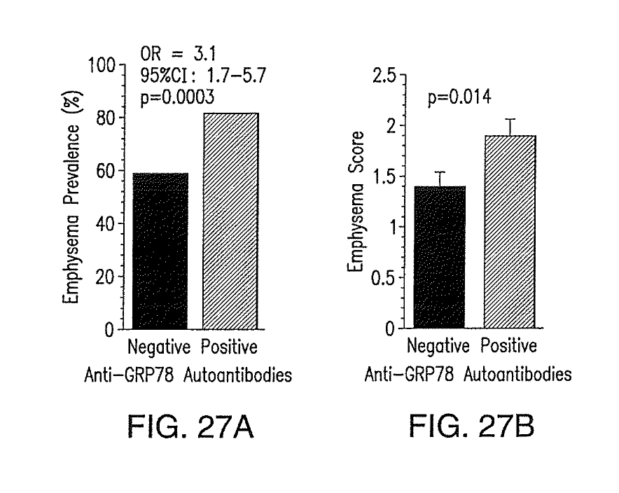

There is a strong association between the presence of anti-GRP78 autoantibodies and emphysema in this cohort (FIG. 27A-B).

We focused particular attention on detection of autoimmune correlates among subjects at risk for COPD, i.e., the smoke controls (SC). We hypothesized a priori that the presence of autoimmune processes in these subjects may be singularly important factors in early disease progression. We further hypothesized that immunopathogenic effects might be more easily detected in subjects with the most subtle or nascent abnormalities, in distinction to patients with extensive histological, radiographic, and physiologic lung disease (or near ubiquitous presence of emphysema, see TABLE 9). Demographic and PFT values of the SC are shown in Table 3. There was a trend for under-representation of HLA DRβ1*11 among the anti-GRP78 positive subjects (SC subjects had HLA typing in the context of previous studies). Abnormalities of HLA allele frequencies (either over- or under-representations) are also a hallmark of autoimmune disorders. Interestingly, we have seen a similar “protective” effect of DRβ1*11 with respect to clinically-relevant autoimmunity to heat shock protein 70 in patients with idiopathic pulmonary fibrosis (IPF).

TABLE 3

| | | Age | | | | | | DR11 |
|---|---|---|---|---|---|---|---|---|
| | n | (yr) | % male* | FEV1 % p | FEV1/FVC | DLCO % p | PY | (%)** |
| Antibody | 27 | 66 ± 1 | 30 | 99 ± 2 | 0.78 ± 0.01 | 76 ± 3 | 62 ± 7 | 11 |
| Pos | | | | | | | | |
| Antibody | 72 | 67 ± 1 | 61 | 98 ± 1 | 0.77 ± 0.01 | 79 ± 2 | 62 ± 3 | 29 |
| Neg | | | | | | | | |

Demographics of Smoke Control (SC) subjects. Antibody Pos and Antibody Neg denote SC subjects with and without anti-GRP78 autoantibodies, respectively. % p=percentages of predicted values; PY=pack years cigarette smoking; DR11=prevalence of HLA Class II allele DRβ1*11; *p 0.005, **p=0.06

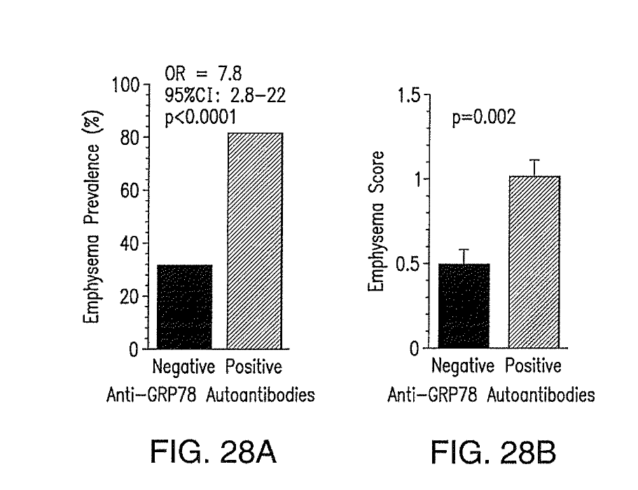

The association between anti-GRP78 autoantibodies and emphysema is greater among the SC (FIG. 28A-B) than for the study population as a whole.

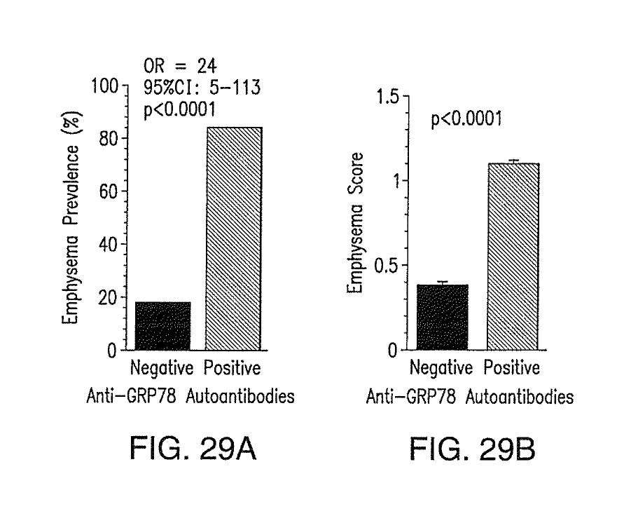

This clinical-immunologic association is even greater in post hoc analyses limited to the female SC subjects (FIG. 29A-B)

Osteoporosis: A clinical association between emphysema and osteoporosis has been well established. The linkage between these syndromes has been postulated to have an immunologic basis. Interestingly, activation of macrophages and increased production of TNF-α are believed to play some role in the etiology of both emphysema and osteoporosis.

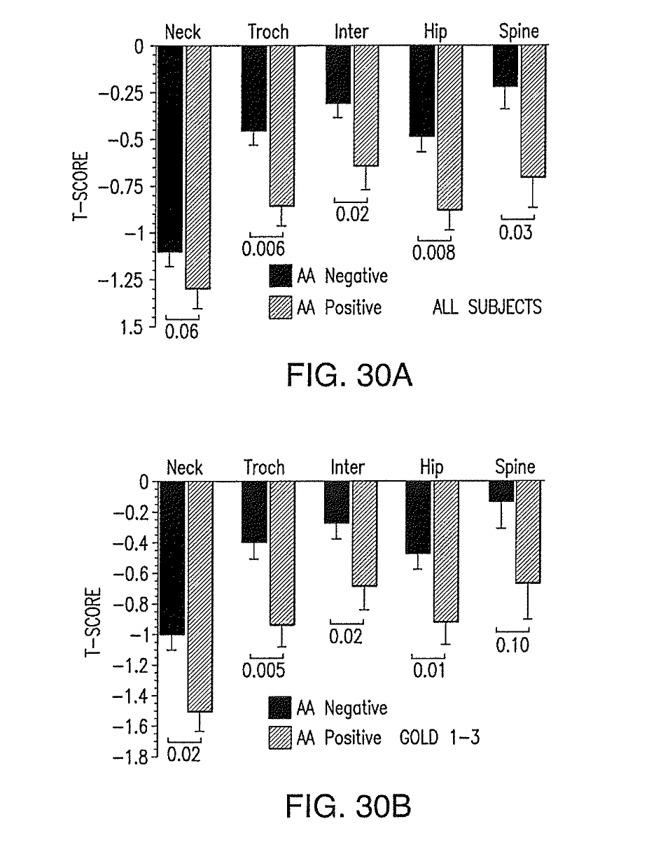

We have found a strong association between the presence of anti-GRP78 autoantibodies and osteoporosis in this cohort (FIG. 30A-B). This relationship is strongest among subjects in this cohort who have COPD (FIG. 30 B).

Multivariate analyses have further shown the association between anti-GRP78 autoreactivity and osteoporosis is independent of emphysema per se.

Data Summary. Findings presented here, showing the presence of GRP78 self-reactivity, presences of the autoantigen, and specific immunopathogenic processes in COPD lungs fulfill criteria of antigen-specific autoimmunity (23-25). Moreover, the strong associations between the autoreactivity and clinical manifestations of two different smoking-associated disorders suggest this immune response plays a casual pathogenic role.

Without being bound to any particular theory, anti-GRP78 autoantibodies may bind to and activate macrophages in lungs and bone (osteoclasts), causing them to elaborate TNF-α and other pro-inflammatory and injurious cytokines that result in emphysema and osteoporosis. Studies to test this hypothesis are ongoing (and preliminary results seem supportive). Based on extrapolations of findings in other patient populations, we believe the anti-GRP78 autoantibodies have pathogenic effects that can mechanistically account for the development of both emphysema and osteoporosis.

### 10.3. REFERENCES

1. Lopez A D, Mathers C D, Ezzati M, Jamison D T, Murray C J. Global and regional burden of disease and risk factors, 2001: systematic analysis of population health data. Lancet 2006; 367:1747-1757 PMID: 16731270
2. Mannino D M COPD: epidemiology, prevalence, morbidity and mortality, and disease heterogeneity. Chest, 2002, 121:121 S-6S PMID: 12010839
3. Chatila W M, Thomashow B M, Minai O A, Criner G J, Make B J. Comorbidities in chronic obstructive pulmonary disease. Proceedings of the American Thoracic Society 2008; 5:549-555. PMID: 18453370
4. Agusti A G N. Systemic effects of chronic obstructive pulmonary disease. Proceedings of the American Thoracic Society 2005; 2:367-370. PMID: 16267364
5. Decramer M, Rennard S, Troosters T, Mapel D W, Giardino N, Mannino D, Wouters E, Sethi S, Cooper C B. Copd as a lung disease with systemic consequences—clinical impact, mechanisms, and potential for early intervention. COPD 2008; 5:235-256. PMID: 18671149
6. McAllister D A, Maclay J D, Mills N L, Mair G, Miller J, Anderson D, Newby D E, Murchison J T, Macnee W. Arterial stiffness is independently associated with emphysema severity in patients with chronic obstructive pulmonary disease. Am J Resp Crit Care Med 2007; 176:1208-1214 PMID: 17885263
7. Sabit R, Bolton C E, Edwards P H, Pettit R J, Evans W D, McEniery C M, Wilkinson I B, Cockcroft J R, Shale D J. Arterial stiffness and osteoporosis in chronic obstructive pulmonary disease. Am J Respir Crit Care Med 2007; 175:1259-1265. PMID: 17363772
8. de Vries F, van Staa T P, Bracke M S, Cooper C, Leufkens H G, Lammers J W. Severity of obstructive airway disease and risk of osteoporotic fracture. Eur Respir J 2005; 25:879-884 PMID: 15863646
9. Bolton C E, Ionescu A A, Shiels K M, Pettit R J, Edwards P H, Stone M D, Nixon L S, Evans W D, Griffiths T L, Shale D J. Associated loss of fat-free mass and bone mineral density in chronic obstructive pulmonary disease. Am J Resp Crit Care Med, 2004; 170:1286-1293 PMID: 15374843
10. Bon J M, Fuhrman C R, Weissfeld J L, Duncan S R, Branch R A, Chang J H C, Leader K L, Our D, Greenspan S L, Sciurba F C. Radiographic Emphysema Predicts Low Bone Mineral Density in a Tobacco Exposed Cohort Am J Respir Crit Care Med. 2011; 183:885-90. PMID: 20935108
11. Wilson D O, Weissfeld J L, Balkan A, Schragin J G, Fuhrman C R, Fisher S N, Wilson J, Leader J K, Siegfried J M, Shapiro S D, Sciurba F C. Association of radiographic emphysema and airflow obstruction with lung cancer. Am J Resp Crit Care Med, 2008; 178:738-744 PMID: 18565949
12. Duncan S R. What is autoimmunity and why is it likely to be important in chronic lung disease? Am J Respir Crit Care Med. 2010 Jan. 1; 181(1):4-5. PMID: 20026750
13. Elkon K, Casali P. Nature and functions of autoantibodies. Nat Clin Pract Rheumatol 2008; 4:491-498. PMID: 18756274
14. Ermann J, Fathman C O. Autoimmune diseases: genes, bugs, and failed regulation. Nat Immunol 2001; 2:759-761 PMID: 11526377
15. Marrack P, Kappler J, Kotzin B L. Autoimmune disease: why and where it occurs. Nat Immunol 2001; 7:899-905 PMID: 11479621
16. Lipsky P E. Systemic lupus erythematosus: an autoimmune disease of B cell hyperactivity. Nat Immunol 2001; 2:764-766 PMID: 11526379
17. Agusti A, MacNee W, Donaldson K, Cosio M. Hypothesis: does COPD have an autoimmune component? Thorax 2003; 58:832-4 PMID: 14514931
18. Buist A S, McBurnie M A, Vollmer W M, Gillespie S, Burney P, Mannino D M, Menezes A M, Sullivan S D, Lee T A, Weiss K B, Jensen R L, Marks G B, Gulsvik A, Nizankowska-Mogilnicka E; BOLD Collaborative Research Group International variation in the prevalence of COPD (the BOLD Study): a population-based prevalence study. Lancet. 2007; 370:741-750 PMID: 17765523
19. Burrows B, Niden A H, Barclay W R, Kasik J E. Chronic obstructive pulmonary disease II. Relationships of clinical and physiological findings to the severity of airways obstruction. Am Rev Respir Dis, 1965, 91:665-678 PMID: 14280940
20. Kurzius-Spencer M, Sherrill D L, Holberg C J, Martinez F D, Lebowitz M D. Familial correlation in the decline of forced expiratory volume in one second. Am J Respir Crit Care Med, 2001, 164:1261-1265 PMID: 11673220
21. Retamales I. Elliott W M. Meshi B, Coxson H O, Pare P D, Sciurba F C, Rogers R M, Hayashi S, Hogg J C. Amplification of inflammation in emphysema and its association with latent adenoviral infection. Am J Respir Critical Care Med. 2001; 164:469-73 PMID: 11500352
22. Perosa F, Prete M, Racanelli V, Dammacco F. CD20-depleting therapy in autoimmune diseases: from basic research to the clinic. J Intern Med. 2010, 267:260-77 PMID: 20201920
23. Erickson S B, Kurtz S B, Donadio J V, Holley K E, Wilson C B, Pineda A A. Use of combined plasmapharesis and immunosuppression in the treatment of Goodpasture's syndrome. Mayo Clin Proc 1979; 54:714-720 PMID: 491763
24. Khosroshahi A, Bloch D B, Deshpande V, Stone J H. Rituximab therapy leads to rapid decline of serum IgG4 levels and prompt clinical improvement in IgG4-related systemic disease. Arthritis Rheum. 2010; [epub ahead of print] PMID: 20191576
25. Keogh K A, Wylam M E, Stone J H, Specks U Induction of remission by B lymphocyte depletion in eleven patients with refractory antineutrophil cytoplasmic antibody-associated vasculitis. Arthritis Rheum. 2005; 52:262-8. PMID: 15641078
26. Feghali-Bostwick C A, Gadgil A S, Otterbein L E, Pilewski J M, Stoner M W, Csizmadia E, Zhang Y, Sciurba F C, Duncan S R. Autoantibodies in patients with chronic obstructive pulmonary disease. Am J Resp Critical Care Med 2008; 177:156-163. PMID: 17975205
27. Greene C M, Low T B, O'Neill S J, McElvaney N G. Anti-proline-glycine-proline or antielastin autoantibodies are not evident in chronic inflammatory lung disease. Am J Respir Crit Care Med. 2010; 181:31-5. PMID: 19762563
28. Nunez B, Sauleda J, Antó J M, Julià M R, Orozco M, Monsó E, Noguera A, Gómez F P, Garcia-Aymerich J, Agusti A. Anti-tissue antibodies are related to lung function in chronic obstructive pulmonary disease. Am J Respir Crit Care Med. 2010 Nov. 19. [Epub ahead of print]. PMID: 21097696
29. Lee S-H, Goswami S, Grudo A, Song L-Z, Bandi V, Goodnight-White S, Green L, Hacken-Bitar J, Huh J, Bakaeen F, Coxson H O, Cogswell S, Storness-Bliss C, Cony D B, Kheradmand K. Antielastin autoimmunity in tobacco smoking-induced emphysema. Nat Med 2007; 13:567-9 PMID: 17450149
30. Cottin V, Fabien N, Khouatra C, Moreira A, Cordier Anti-elastin autoantibodies are not present in combined pulmonary fibrosis and emphysema. Eur Resp J 2009; 33:219-221 PMID: 19118235
31. Global Initiative for Chronic Obstructive Lung Disease. Global strategy for the diagnosis, management, and prevention of chronic obstructive pulmonary disease. www.goldcopd.com. Date last assessed Mar. 10, 2010
32. Mahler D, Wells C. Evaluation of clinical methods for rating dyspnea. Chest 1988; 93, 580-586 PMID: 3342669
33. Jones, P. W., F. H. Quirk, C. M. Baveystock, P. Littlejohns P. A self-complete measure of health status for chronic airflow limitation: the St. George's Respiratory Questionnaire. Am Rev Respir Dis. 1992; 145:1321-1327 PMID: 1595997
34. Bon J M, Zhang Y, Duncan S R, Pilewski J M, Zaldonis D, Zeevi A, McCurry K R, Greenspan S L, Sciurba F C. Plasma inflammatory mediators associated with bone metabolism in COPD. COPD 2010; 7:186-91 PMID: 20486817
35. Bon J, Fuhrman C R, Weissfeld J L, Duncan S R, Branch R A, Chang C C, Zhang Y, Leader J K, Gur D, Greenspan S L, Sciurba F C. Radiographic emphysema predicts low bone mineral density in a tobacco-exposed cohort.
36. Gilani S R, Vuga L J, Lindell K O, Gibson K F, Xue J, Lindsay E K, Kaminski N, Valentine V G, George M P, Steele C, Duncan S R. CD28 down-regulation on circulating CD4 T-cells is associated with poor prognoses of patients with idiopathic pulmonary fibrosis. Plos One 2010; 5:e8959 PMID: 20126467
37. Gonzalez-Grwonow M, Cuchacovich M, Lianas C, Urzua C, Gawdi G, Pizzo S V. Prostate cancer cell proliferation in vitro is modulated by antibodies against glucose-regulated protein 78 isolated from patient serum. Cancer Res 2006; 66:11424-11431 PMID: 17145889
38. Lu M-C, Lai N-S, Yu H-C, Huang H-B, Hsieh S-C, Yu C-L. Anticitrullinated protein antibodies bind surface-expressed citrullinated Grp78 on monocyte/macrophages and stimulate tumor necrosis factor alpha production. Arth Rheum 2010, 62:1213-1223 PMID: 20213805
39. Gonzalez-Gronow M, Selim M A, Papalas J, Pizzo S V. GRP78: a multifunctional receptor on the cell surface. Antioxid Redox Signal. 2009; 11:2299-306. PMID: 19331544
40. Chan C-P, Siu K-L, Chin K-T, Yuen K-Y, Zheng B, Jin D-Y. Modulation of the unfolded protein response by the severe acute respiratory syndrome coronavirus spike protein. J Virol 2006; 80:9279-9287 PMID: 16940539
41. Kelsen S G, Duan X, Rang J, Perez O, Liu C, Merali S. Cigarette smoke induces an unfolded protein response in the human lung. Am J Resp Cell Mol Bio. 2008; 38: 541-550 PMID: 18079489
42. Jorgenson E, Stinson A, Shan L, Yang J, Gietl D, Albion A P. Cigarette smoke induces endoplasmic reticulum stress and the unfolded protein response in normal and malignant human lung cells. BMC Cancer 2008; 8:229-259 PMID: 18694499
43. Ravindran S, Narayanan K, Eapen A S, Hao J, Ramachandran A, Blond S, George A. Endoplasmic reticulum chaperone protein GRP-78 mediates endocytosis of dentin matrix protein 1. J Biol Chem 2008; 283:29658-29670 PMID: 18757373
44. Misra U K, Gonzalez-Gronow M, Gawdi G, Hart J P, Johnson C E, Pizzo S V. The role of Grp 78 in alpha 2-macroglobulin-induced signal transduction. Evidence from RNA interference that the low density lipoprotein receptor-related protein is associated with, but not necessary for, GRP 78-mediated signal transduction.) Biol Chem. 2002; 277:42082-42087. PMID: 12194978
45. Misra U K, Gonzalez-Gronow M, Gawdi G, Pizzo S V. The role of MTJ-1 in cell surface translocation of GRP78, a receptor for alpha 2-macroglobulin-dependent signaling. J. Immunol. 2005; 174:2092-2097. PMID: 15699139
46. Purcell A W, Todd A, Ginoshita G, Lynch T A, Keech C I, Gething M J, Gordon T P. Association of stress proteins with autoantigens: a possible mechanism for triggering autoimmunity. Clin Exp Immunol 2003; 132:193-200. PMID: 12699405
47. Feghali-Bostwick C A, Tsai C G, Valentine V G, Kantrow S, Stoner M W, Pilewski J M, Gadgil A, George M P, Gibson K F, Choi A M, Kaminski N, Zhang Y, Duncan S R. Cellular and humoral autoreactivity in idiopathic pulmonary fibrosis. J Immunol 2007; 179:2592-2599 PMID: 17675522
48. Monaco C, Andreakos E, Kiriakidis S, Feldman M, Paleolog C. T-cell-mediated signaling in immune, inflammatory and angiogenic processes: the cascade of events leading to inflammatory diseases. Curr Drug Targets Inflamm Allergy 2004; 3:35-42. PMID: 15032640
49. Parker D C. T-cell dependent B-cell activation. Annu Rev Immunol 1993; 11:331-340 PMID: 8476565
50. Zhu X, Gadgil A S, Givelber R, George M P, Stoner M W, Sciurba F C, Duncan S R. Peripheral T-cell functions correlate with the severity of chronic obstructive pulmonary disease. J. Immunol 2009; 182:3270-3277. PMID: 19234225

Various publications are cited herein, the contents of which are hereby incorporated by reference in their entireties.

What is claimed is:

1. A method of diagnosing a chronic lung disease, selected from the group consisting of idiopathic pulmonary fibrosis, chronic obstructive pulmonary disease, and emphysema, in a subject, comprising determining whether the subject has developed an autoimmune response to a Hsp70 autoantigen, where the presence of the autoimmune response supports the diagnosis of the chronic lung disease.

2. The method of claim 1, where the determination of whether the subject has developed the autoimmune response comprises determining whether a sample from the subject contains antibodies specific for the Hsp70 autoantigen.

3. The method of claim 1, where the determination of whether the subject has developed the autoimmune response comprises determining whether a sample from the subject contains T cells that are capable of being activated by the Hsp70 autoantigen.

4. The method of claim 1, where the Hsp70 autoantigen is Hsp70-1a.

5. The method of claim 1, where the Hsp70 autoantigen is Grp78.

6. The method of claim 1, further comprising determining whether the subject exhibits the DRB1*15 genotype, where the presence of said genotype further supports the diagnosis of the chronic lung disease.

7. A method of determining the prognosis of a subject suffering from a chronic lung disease, selected from the group consisting of idiopathic pulmonary fibrosis, chronic obstructive pulmonary disease, and emphysema, comprising determining whether the subject has developed an autoimmune response to a Hsp70 autoantigen, where the presence of the autoimmune response indicates a poorer prognosis.

8. The method of claim 7, where the determination of whether the subject has developed the autoimmune response comprises determining whether a sample from the subject contains antibodies specific for the Hsp70 autoantigen.

9. The method of claim 7, where the determination of whether the subject has developed the autoimmune response comprises determining whether a sample from the subject contains T cells that are capable of being activated by the Hsp70 autoantigen.

10. The method of claim 7, where the Hsp70 autoantigen is Hsp70-1a.

11. The method of claim 7, where the Hsp70 autoantigen is Grp78.

12. A method of treating a subject suffering from a chronic lung disease, selected from the group consisting of idiopathic pulmonary fibrosis, chronic obstructive pulmonary disease, and emphysema, comprising determining whether the subject has developed an autoimmune response to a Hsp70 autoantigen, and, where the autoimmune response is present, recommending a further interventional step selected from the group consisting of lung transplant, lung biopsy (transplant recipient only), closer observation and serial PFT testing, and augmentation or addition or substitution of immunosuppressive medication.

13. The method of claim 12, where the determination of whether the subject has developed the autoimmune response comprises determining whether a sample from the subject contains antibodies specific for the Hsp70 autoantigen.

14. The method of claim 12, where the determination of whether the subject has developed the autoimmune response comprises determining whether a sample from the subject contains T cells that are capable of being activated by the Hsp70 autoantigen.

15. The method of claim 12, where the Hsp70 autoantigen is Hsp70-1a.

16. The method of claim 12, where the Hsp70 autoantigen is Grp78.

17. A method of diagnosing osteoporosis in a subject, comprising determining whether the subject has developed an autoimmune response to a Hsp70 autoantigen, where the presence of the autoimmune response supports the diagnosis of osteoporosis.

18. The method of claim 17, where the determination of whether the subject has developed the autoimmune response comprises determining whether a sample from the subject contains antibodies specific for the Hsp70 autoantigen.

19. The method of claim 17, where the determination of whether the subject has developed the autoimmune response comprises determining whether a sample from the subject contains T cells that are capable of being activated by the Hsp70 autoantigen.

20. The method of claim 17, where the Hsp70 autoantigen is Hsp70-1a.

21. The method of claim 17, where the Hsp70 autoantigen is Grp78.

22. A method of treating a subject suffering from osteoporosis, comprising determining whether the subject has developed an autoimmune response to a Hsp70 autoantigen, and, where the autoimmune response is present, recommending treatment with an agent selected from the group consisting of: bisphosphonate drug, raloxifene, calcitonin, teriparatide, or hormone therapy.

23. The method of claim 22, where the determination of whether the subject has developed the autoimmune response comprises determining whether a sample from the subject contains antibodies specific for the Hsp70 autoantigen.

24. The method of claim 22, where the determination of whether the subject has developed the autoimmune response comprises determining whether a sample from the subject contains T cells that are capable of being activated by the Hsp70 autoantigen.

25. The method of claim 22, where the Hsp70 autoantigen is Hsp70-1a.

26. The method of claim 22, where the Hsp70 autoantigen is Grp78.

27. A method of determining the prognosis of a subject suffering from a chronic lung disease, selected from the group consisting of idiopathic pulmonary fibrosis, chronic obstructive pulmonary disease, and emphysema, comprising determining whether the subject has the DRB1*11 genotype, where the presence of said genotype indicates a better prognosis.

28. A method of determining the prognosis of a subject suffering from idiopathic pulmonary fibrosis, comprising determining whether the subject has the rs1061581 allele, where the presence of said allele indicates a better prognosis.
